(19) United States
(12) Patent Application Publication
(10) Pub. No.: US 2025/0352668 A1
(43) Pub. Date: Nov. 20, 2025

(54) AAV VECTOR ENCODING DIAMINE OXIDASE AND USES THEREOF

(71) Applicant: OYSTER POINT PHARMA, INC., Princeton, NJ (US)

(72) Inventors: Eric C. CARLSON, Princeton, NJ (US); Jeffrey Alan NAU, Princeton, NJ (US)

(21) Appl. No.: 18/871,469

(22) PCT Filed: Jun. 5, 2023
(86) PCT No.: PCT/US2023/067948

§ 371 Date: Dec. 3, 2024

Related U.S. Application Data
(60) Provisional application No. 63/348,856, filed on Jun. 3, 2022.
(60) Provisional application No. 63/383,026, filed on Nov. 9, 2022.

(51) Int. Cl. A61K 48/00; A61K 9/00; A61K 38/00; A61P 37/08; C07K 14/005; C12N 9/06; C12N 15/86

(52) U.S. Cl. CPC A61K 48/005; A61K 9/0048; A61K 48/0075; A61K 48/0083; A61P 37/08; C07K 14/005; C12N 9/0022; C12N 15/86; A61K 38/00; C12N 2750/14122; C12N 2750/14143; C12N 2830/48; C12N 2830/50; C12Y 104/03022

(57) ABSTRACT

Provided are recombinant adeno-associated virus (rAAV) vectors, methods of treating a condition (e.g., an ocular condition), pharmaceutical compositions, and other compositions and methods, in which the rAAV vector comprises a polynucleotide encoding diamine oxidase (DAO1). Methods of treatment may include administration to the lacrimal gland.

### CROSS-REFERENCE TO RELATED APPLICATIONS

This application claims the benefit of the priority dates of U.S. Provisional Patent Application Nos. 63/383,026, filed Nov. 9, 2022, and 63/348,856, filed Jun. 3, 2022, the disclosures of which are incorporated by reference herein in their entirety.

### SEQUENCE LISTING

This application incorporates by reference in its entirety the Sequence Listing XML file entitled “037525-00558.xml (71 KB), which was created on May 24, 2023, and filed electronically herewith.

### BACKGROUND

Medical treatment of ocular conditions that affect the ocular surface, such as allergic conjunctivitis that include perennial and/or seasonal rhinoconjunctivitis, atopic keratoconjunctivitis, giant papillary conjunctivitis, and vernal (VKC) or allergic keratoconjunctivitis are known. These medical treatments include topical eye drops, oral pharmaceuticals, and others.

Despite available treatments conjunctivitis remain a challenge for eye care practitioners (ophthalmologists and optometrists) and therapeutic strategies focus on the severity of the disease. Treatments for less severe cases may include antihistamines, mast cell stabilizers, anti-inflammatory, and/or steroid topical eye drops. For more severe cases oral antihistamines or oral leukotriene inhibitor medications may be employed.

Delivery of antihistamines, mast cell stabilizers, calcineurin inhibitors, anti-inflammatory, and/or steroid topical eye drops for treatment of the conjunctivitis currently relies on repeated application of eye drops, which in many severe cases do not provide adequate treatment outcomes, such as in vernal or atopic keratoconjunctivitis where, even with treatment, it can result in corneal ulceration, cataract formation, or glaucoma.

### SUMMARY OF INVENTION

In view of the shortcomings of current medical treatment options for ocular surface disorders, there exists a need for improved methods for delivering antihistamine therapy to the ocular surface, e.g., without the need for repeated topical applications that can be challenging or impossible for some patients and can result in poor patient compliance. In addition, pulsed administration of antihistamine therapy may only provide temporary or “episodic” relief and may not be suitable for chronic conditions. The present disclosure provides recombinant constructs and methods that address these and other needs in some aspects, in addition to providing various other benefits as described herein and illustrated by the accompanying figures.

In some aspects, the disclosure provides adeno-associated virus (AAV) vectors for expression of diamine oxidase (“DAO1”; alternatively abbreviated as “DAO” herein) in the eye following in vivo administration.

In one aspect, the disclosure provides a recombinant adeno-associated viral (rAAV) vector comprising an AAV capsid and an expression cassette, the expression cassette comprising a polynucleotide encoding diamine oxidase, also known as DAO1, DAO or histaminase, operatively linked to a promoter. In some embodiments, the polynucleotide encodes a protein that is at least 95% identical to SEQ ID NOs: 1 or 2. In some embodiments, the polynucleotide comprises a nucleotide sequence that is at least 95% identical to a nucleotide sequence selected from SEQ ID NOs: 29-31. In some embodiments, the polynucleotide comprises a nucleotide sequence selected from SEQ ID NOs: 29-31.

In another aspect, the disclosure provides a composition comprising an rAAV vector, wherein the rAAV vector comprises: (a) an AAV capsid, and (b) an expression cassette, wherein the expression cassette comprises a polynucleotide comprising a nucleotide sequence sharing at least 95% identity to a nucleotide sequence selected from SEQ ID NOs: 29-31, and wherein the polynucleotide is linked to a promoter.

In some embodiments, the promoter is a CMV promoter. In some embodiments, the expression cassette comprises a CMV enhancer. In some embodiments, the promoter is a CAG promoter. In some embodiments, the expression cassette comprises a polyadenylation (polyA) sequence. In some embodiments, the polyA sequence is a BGH polyA sequence. In some embodiments, the expression cassette comprises a Woodchuck Hepatitis Virus Posttranscriptional Regulatory Element (WPRE). In some embodiments, the expression cassette comprises a Kozak sequence. In some embodiments, the expression cassette is flanked by two inverted terminal repeats (ITRs). In some embodiments, the ITRs are AAV2 ITRs.

In some embodiments, the expression cassette comprises a nucleotide sequence that shares at least 90%, 95%, 96%, 97%, 98%, 99%, or 100% identity to SEQ ID NO: 23.

In some embodiments, the AAV capsid comprises a VP3 that shares at least 95%, 98%, or 100% identity with AAV2 VP3 (SEQ ID NO: 8), AAV5 VP3 (SEQ ID NO: 10), AAV8 VP3 (SEQ ID NO: 12), or AAV9 VP3 (SEQ ID NO:14). In some embodiments, the AAV capsid comprises a VP3 that shares at least 95%, 98%, or 100% identity with AAV9 VP3 (SEQ ID NO: 14).

In yet another aspect, the disclosure provides a composition comprising an rAAV vector, wherein the rAAV vector comprises: (a) an AAV2, AAV5, AAV8, or AAV9 capsid, and (b) an expression cassette, wherein the expression cassette comprises a polynucleotide comprising a nucleotide sequence sharing at least 95% identity to a nucleotide sequence selected from SEQ ID NOs: 29-31, and wherein the polynucleotide is linked to a promoter.

In some embodiments, the expression cassette comprises a polynucleotide sequence sharing at least 95% identity to SEQ ID NO: 23.

In some embodiments, the AAV capsid is AAV2. In some embodiments, the AAV capsid is AAV5. In some embodiments, the AAV capsid is AAV9.

In one aspect, the disclosure provides a pharmaceutical composition comprising an rAAV vector described herein or a composition described herein (e.g., a polypeptide comprising DAO1 or a fragment thereof), and a pharmaceutically acceptable carrier.

In some embodiments, the pharmaceutical composition comprises about 1×10⁵ to about 1×10¹⁶ genome copies per milliliter of the rAAV vector. In some embodiments, the pharmaceutical composition comprises about 1×10¹² to about 6.2×10¹² genome copies per milliliter of the rAAV vector.

In some embodiments, a pharmaceutical composition may comprise an rAAV vector (or any other vector described herein, such as a plasmid configured to express DAO1) in an amount or at a concentration sufficient to result in the expression of 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 μg/mL of DAO1, or a concentration within a range defined by any pair of the foregoing values, in a tear film of a subject. Expression may be measured, e.g., 1, 2, 3, 4, 5, 6, 7, 8, 9, 10, 11, 12, 13, 14, 15, 16, 17, 18, 19, 20, 21, 22, or 23 hours after administration, or after a longer duration of time, such as after 1, 2, 3, 4, or 5 days.

In some embodiments, a pharmaceutical composition may comprise 0.25, 0.50, 0.75, 1.00, 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, or 6.00 μg of a polypeptide comprising DAO1 (or a fragment thereof), or an amount within a range defined by any pair of the foregoing values. In some embodiments, the pharmaceutical composition may further comprise one or more excipients, carriers, and/or diluents suitable for administration to an eye of a human or animal subject.

In some embodiments, the pharmaceutical composition is formulated for administration into the lacrimal gland. In some embodiments, the pharmaceutical composition is formulated for administration onto the ocular surface. In some embodiments, the pharmaceutical composition is formulated for use, or adaptable for use, in the treatment of an ocular disease, disorder, or condition.

In some aspects, the disclosure provides a method for treating a condition in a subject in need thereof, the method comprising administering an effective amount of a pharmaceutical composition described herein to an eye of the subject.

In some embodiments, the pharmaceutical composition is delivered to an ocular secretory gland of the subject. In some embodiments, the pharmaceutical composition is delivered to the lacrimal gland. In some embodiments, the cells within the lacrimal gland are acinar cells. In some embodiments, the cells within the lacrimal gland are ductile epithelial cells.

In some embodiments, the cells within the lacrimal gland are transduced by the rAAV vector. In some embodiments, the transduced cells within the lacrimal gland express an effective amount of DAO1 into the tear film and optionally onto the ocular surface of the subject. In some embodiments, the pharmaceutical composition is delivered to an accessory lacrimal gland (e.g., to Krause's glands, Wolfring's glands, and/or Popov's gland).

In some embodiments, about 1×10⁹ to about 1×10¹⁰, about 1×10¹⁰ to about 1×10¹¹, about 1×10¹¹ to about 1×10¹², about 1×10¹² to about 1×10¹³, or about 1×10¹³ to about 1×10¹⁵ genome copies of the rAAV vector are administered.

In some embodiments, the condition is an ocular condition. In some embodiments, the condition or the ocular condition is associated with increased histamine production and/or increased histamine signaling. In some embodiments, the condition or the ocular condition is an inflammatory condition. In some embodiments, the condition or the ocular condition is an autoimmune condition. In some embodiments, the condition or the ocular condition is an allergic condition. In some embodiments, the condition or the ocular condition is an allergic reaction to a therapeutic agent. In some embodiments, the condition or the ocular condition is an allergic reaction to a microbial infection. In some embodiments, the condition or the ocular condition is vernal keratoconjunctivitis. In some embodiments, the condition or the ocular condition is atopic keratoconjunctivitis. In some embodiments, the condition or the ocular condition is seasonal or perennial allergic conjunctivitis.

In some embodiments, the administration results in expression of DAO1 in the cells of the lacrimal gland and/or an accessory lacrimal gland. In some embodiments, the administration results in secretion of DAO1 into the tear film. In some embodiments, secretion of DAO1 into the tear film is stimulated by administration of an electrical stimulus, mechanical stimulus, ultrasound stimulus, and/or a drug. An example of an electrical stimulus is an intranasal stimulator such as the TrueTear® Intranasal Tear Neurostimulator. An example of a mechanical stimulus is oscillatory energy provided by a device such as the iTEAR® 100. An example of an ultrasound stimulus is application of a neuromodulation device, like the iTear system (Olympic Ophthalmics) developed to treat dry eye disease. In some embodiments, the drug that stimulates secretion of DAO1 into the tear film is a cholinergic agonist (e.g., pilocarpine or cevimeline). In some embodiments, the drug is a nicotinic acetylcholine receptor (nAChR) agonist (e.g., varenicline). In some embodiments, the drug is a secretagogue or mucoprotective agent (e.g., diquafosol, rebamipide, or ecabet). In some embodiments, the drug that stimulates secretion of DAO1 into the tear film is administered into the eye. In some embodiments the drug, such as the cholinergic agonist, is administered orally. In some embodiments, secretion of DAO1 into the tear film is stimulated by a drug, such as a cholinergic agonist or nAChR agonist, administered into the nasal cavity. In some aspects, DAO1, or an rAAV vector, plasmid, or any other construct configured to express DAO1 described herein, may be administered to a subject as a co-therapy in combination with a tear-increasing stimulus and/or drug (e.g., those described in this paragraph). In some aspects, such co-therapies may follow any protocol, or use any components or parameters, described in International Patent Application Pub. No. WO 2022/235786, the entire contents of which is incorporated herein.

In some embodiments, the administration results in an improvement of one or more symptoms of the ocular condition. In some embodiments, the symptom is selected from the group consisting of itching, swelling, tearing, and redness. In some embodiments, the administration results in an improvement of 0.5 points, 1 point, 1.5 points, 2 points, 2.5 points, 3 point, 3.5 points, or 4 points on the Conjunctival Itching Grading Scale. In some embodiments, the administration results in an improvement of 1 point, 2 points, 3 point, or 4 points on the Conjunctival Redness Assessment Grading Scale. In some embodiments, the administration results in an improvement of 1 point, 2 points, 3 point, or 4 points on the Ocular Tearing Score Grading Scale. In some embodiments, the administration results in an improvement of about 10% to about 20%, about 20% to about 30%, about 30% to about 40%, about 40% to about 50%, about 50% to about 60%, about 60% to about 70%, about 70% to about 80%, about 80% to about 90%, or more than about 95% in at least one area on the Corneal Staining Grading Scale.

In some embodiments, the improvement is measured about 1 months, about 2 months, about 3 months, about 4 months, about 5 months, about 6 months, about 9 months, or about 12 months after the administration. In some embodiments, the improvement persists for at least 3 months, at least 6 months, at least 9 months, at least 12 months, at least 15 months, at least 18 months, at least 24 months, at least 3 years, at least 4 years, or at least 5 years after the administration.

In some embodiments, the method further comprises administering one or more additional therapeutic agents. In some embodiments, the additional therapeutic agent is an agent that increases tear production. In some embodiments, the additional therapeutic agent is an agent that increases tear production is a cholinergic agonist. In some embodiments, the subject is human.

In some embodiments, the disclosure provides DAO1, an rAAV or other vector configured to express DAO1, a composition, or a pharmaceutical composition described herein for use in a method of treating a condition in a subject in need thereof comprising administering an effective amount of the pharmaceutical composition to the eye of the subject.

In some embodiments, the disclosure provides DAO1, an rAAV or other vector configured to express DAO1, a composition, or a pharmaceutical composition described herein for use in the manufacture of a medicament for treating a condition in a subject in need thereof.

In some embodiments, the disclosure provides a kit comprising DAO1, an rAAV or other vector configured to express DAO1, or a composition described herein, and a pharmaceutically acceptable carrier, and instructions for use in treating a condition in a subject.

In some embodiments, the instructions comprise administering the pharmaceutical composition to the eye of the subject.

In some embodiments, the disclosure provides a kit comprising DAO1, an rAAV or other vector configured to express DAO1, or a composition described herein, and a pharmaceutically acceptable carrier, and instructions for use in treating a condition associated with histamine production and/or increased histamine signaling in a subject. In some embodiments, the instructions comprise administering the pharmaceutical composition to the eye of the subject.

In some embodiments, the disclosure provides a kit comprising DAO1, an rAAV or other vector configured to express DAO1, or a composition described herein, and a pharmaceutically acceptable carrier, and instructions for use in treating an autoimmune condition in a subject. In some embodiments, the instructions comprise administering the pharmaceutical composition to the eye of the subject.

In some embodiments, the disclosure provides a kit comprising DAO1, an rAAV or other vector configured to express DAO1, or a composition described herein, and a pharmaceutically acceptable carrier, and instructions for use in treating an allergy condition in a subject. In some embodiments, the instructions comprise administering the pharmaceutical composition to the eye of the subject.

### BRIEF DESCRIPTION OF FIGURES

FIG. 1A shows a vector map of an rAAV expression cassette with inverted terminal repeats (“ITRs”), a promoter, and DAO1 polynucleotide elements.

FIG. 1B provides a schematic depicting the elements positioned between the ITRs of the rAAV expression cassette, including from 5′ to 3′: a cytomegalovirus (“CMV”) enhancer/promoter, an N-terminal secretion signal, a nucleotide sequence encoding DAO1, a woodchuck hepatitis virus post-translational regulatory element (“WPRE”), and polyadenylation (“pA”) signal.

FIGS. 2A and 2B show examples of delivery of a viral vector to the lacrimal gland of a human subject.

FIG. 3 shows an illustration of the Corneal Staining Grading Scale, which is a grading diagram of the division of the corneal surface based on the NEI/Industry Workshop scale.

FIG. 4A provides images obtained by confocal fluorescence microscopy of 293T cells transfected with an AAV plasmid encoding DAO1 at 20× magnification (top row) or 63× magnification (middle row) as compared to non-transfected control 293T cells at 20× magnification (bottom row). The left-most column shows detection of fluorescent signal for an AF488-labeled secondary antibody used to detect labeling with a primary anti-DAO1 antibody, the middle column shows fluorescence signal for nuclear staining using DAPI, and the right-most column shows an overlay of the AF488 and DAPI fluorescent signals.

FIG. 4B provides images obtained by confocal fluorescence microscopy of 293 T cells transfected with an AAV plasmid encoding DAO1 at 20× magnification (middle row) or 63× magnification (bottom row) compared to non-transfected control 293T cells at 20× magnification (top row). The right-most column shows fluorescence signal for nuclear staining using DAPI, the middle column shows of fluorescent signal for an AF488-labeled secondary antibody used to detect labeling with a primary anti-DAO1 antibody, and the right-most column shows and overlay of the AF488 and DAPI fluorescent signals.

FIG. 4C provides images obtained by confocal fluorescence microscopy of PANC-1 cells transfected with an AAV plasmid encoding DAO1 at 20× magnification (bottom row) as compared to non-transfected control cells at 40× magnification and labeled with a secondary antibody (top row) or at 20× magnification and unlabeled (middle row). Fluorescent signals are as described in FIG. 4B.

FIG. 4D provides images obtained by confocal fluorescence microscopy of PANC-1 cells transfected with an AAV plasmid encoding DAO1 at 40× magnification (top row) as compared to non-transfected control cells at 20× magnification (bottom row). Fluorescent signals are as described in FIG. 4B.

FIG. 5A shows an image of a Western blot of whole cell lysates harvested from 293T cells at 48 hours following transient transfection with AAV.DAO1 (lane labeled “AAV-DAO1”) as compared to control non-transfected 293T cells (lane labeled “293T”), with detection of DAO1 using a rabbit anti-DAO1 antibody. An arrow indicates the protein band corresponding to the DAO1 polypeptide.

FIG. 5B shows an image of a Western blot of recombinant human DAO1 polypeptide with detection of DAO1 using a mouse anti-his antibody.

FIGS. 5C-5D shows an image of a Western blot for detection of DAO1 in whole cell lysates harvested from 293T cells at 24 hours following transient transfection with AAV.DAO1 (lane labeled “AAV-DAO”) as compared to control non-transfected 293T cells (lane labeled “293T”) and recombinant human DAO1 polypeptide (lane labeled “Recombinant DAO”). An arrow indicates the protein band corresponding to the DAO1 polypeptide (FIG. 5C). GAPDH western blot (15 g/well) served as the loading control (FIG. 5D).

FIG. 5E shows an image of a Western blot for detection of DAO1 in conditioned media obtained from 293T cells transiently transfected with AAV.DAO1 (“AAV.DAO”) or non-transfected (“293T”) at 2, 4, and 6 hours following a fresh media change. An arrow indicates the protein band corresponding to the DAO1 polypeptide.

FIGS. 6A-6B show data generated using a colorimetric functional activity assay to measure DAO1 substrate cleavage. DAO1 cleaves a first substrate to yield H₂O₂ (hydrogen peroxide) that reacts with a second substrate to signal that is detected using fluorescence spectroscopy (excitation wavelength of 535 nm and emission wavelength of 587 nm). FIG. 6A shows a calibration curve generated by measuring fluorescence signal of the second substrate as a function of concentration of H₂O₂. FIG. 6B shows a graph quantifying H₂O₂ generated by activity of DAO1 present in media or cell lysate of 293T cells transiently transfected with AAV plasmid encoding DAO1 as compared to activity of media of non-transfected control cells.

FIG. 6C provides a graph quantifying H₂O₂ generated at 30, 60, and 90 minutes by activity of DAO1 present in combined conditioned media and whole cell extract obtained from 293T cells transiently transfected with AAV plasmid encoding DAO1 or non-transfected control cells. Recombinant DAO1 was used as positive control.

FIG. 7 shows a schematic diagram depicting the elements between the ITRs of an AAV plasmid. The plasmid encodes EGFP linked to a secretion signal at its N-terminus (“secEGFP”) under control of a CMV promoter. The woodchuck hepatitis virus post-translational regulatory element (WPRE) serves to increase transgene expression and is proximal to the bovine growth hormone polyadenylation (pA) signal.

FIGS. 8A-8K are images of lacrimal tissue stained with anti-eGFP antibody. Lacrimal glands were dosed with rAAV vectors containing an expression cassette with eGFP transgene by intralacrimal injection. Lacrimal tissue was stained with anti-eGFP antibody to assess eGFP expression. Black arrows indicate staining showing eGFP expression.

FIG. 9 provides images of porcine lacrimal gland that received an injection of AAV-secEGFP (AAV2 or AAV9 serotype) and was harvested on Day 103 and fixed in paraffin. IHC of 5 μM sections was performed using anti-GFP antibody and DAPI (nuclear) counterstain. Images were captured using a confocal microscope at 100× magnification. Negative control animals received no injection.

FIG. 10 provides an image of porcine lacrimal gland that received an injection of AAV9-secEGFP and was harvested on Day 103 and fixed in paraffin. IHC of 5 μM sections was performed using anti-GFP antibody and DAPI (nuclear) counterstain. In addition to lacrimal gland acinar cells, ductile epithelial cells appear to be transduced by AAV9 (white arrows).

FIG. 11 provides an image of porcine lacrimal gland that received an injection of AAV was harvested at Day 103 and fixed in paraffin. H&E staining of 5 μM paraffin sections at 100× reveal no inflammatory infiltrate, macro, or micro abnormalities.

FIG. 12 provides a schematic of a treatment schedule for administering AAV encoding a model protein via injection into the lacrimal gland of pigs in combination with administration of OC-01 (Varenicline) by nasal spray. Time points for tear collection and termination of the study are indicated.

FIG. 13 shows a calibration curve generated by measuring fluorescence signal of the second substrate as a function of concentration of histamine. This calibration curve was used to evaluate the histamine degradation kinetics of recombinant porcine diamine oxidase (FIGS. 14-15) and histamine degradation by conditioned medium obtained from non-transfected control 293T cells and 293T cells transfected with a plasmid engineered to express DAO1 (FIGS. 16-17).

FIGS. 14-15 provide graphs showing the histamine degradation kinetics of recombinant porcine diamine oxidase at 4 (FIG. 14) and 24 hours (FIG. 15).

FIGS. 16-17 provide graphs showing histamine degradation by conditioned medium obtained from non-transfected control 293T cells and 293T cells transfected with a plasmid engineered to express DAO1, at 4 (FIG. 16) and 24 hours (FIG. 17).

### DETAILED DESCRIPTION

The present disclosure provides methods of treating an ocular condition in a subject in need thereof. Such methods comprise, e.g., the expression of diamine oxidase (DAO1) and subsequent secretion from the lacrimal gland which may then be transferred to and combined with other components of the tear film onto the surface of the eye. In healthy subjects, the primary function of DAO1 is to break down excess histamine in the body. DAO1 is scarcely present in the blood circulation of humans, although it has been shown to increase substantially in pregnant women, suggesting a protective mechanism against adverse histamine. It has also been shown that in subjects with histamine intolerance and a number with ocular conjunctivitis, there is a disequilibrium between accumulated histamine and histamine degradation. Following a variety of noxious stimuli, excess histamine can be released from basophil granules and mast cells, thus creating tissue edema and starting a cascade of events leading to inflammation and allergic response. Elevated histamine levels in the tear film are a hallmark of perennial and/or seasonal rhinoconjunctivitis, atopic keratoconjunctivitis, giant papillary conjunctivitis, and vernal or allergic keratoconjunctivitis. Histamine induces itching, redness and lacrimation through the activation of H1 receptors on blood vessels and nociceptive nerves. In severe cases, the patient can develop a visual impairment due to corneal damage, cataract, or glaucoma.

As disclosed herein, AAV-based delivery of transgenes to the eye provides a method to treat ocular disorders. rAAV vectors have distinct advantages for transgene delivery. A transgene delivered by an rAAV vector is likely to be incorporated into the genome of the transduced cell, allowing potential long-term expression of the transgene product. In addition, rAAV vectors may be less immunogenic as compared to other viral delivery vectors, such as adenovirus. AAV-based delivery vectors using subretinal and intravitreal injection to transduce cells in the posterior segment of the eye are described and show efficacy in vivo (U.S. Pat. No. 10,308,957; Petrs-Silva et al., Mol Ther. 19:293-301 (2011); Rodriques et al., Pharm Res. 36:29 (2019)).

AAV serotypes used in AAV-based delivery of transgene to the eye include AAV1, AAV2, AAV4, AAV5, AAV6, AAV7, AAV8, and AAV9 used to deliver transgene (Lebherz et al. J Gene Med.; 10(4): 375-382 (2008)). AAV serotypes used in AAV-based delivery of a transgene to the lacrimal gland include AAV2, AAV4, AAV5, AAV5w8, AAV x5, AAV 9, AAV 12, and BAAV (Rocha et al., Invest Ophthalmol Vis Sci. 52:9567-9572 (2011)).

In some embodiments, an rAAV vector is provided for the expression of DAO1. In some embodiments, cells of the at least one eye and/or lacrimal gland are transduced with the rAAV vector of the disclosure. The lacrimal glands are the major source of tear fluid responsible for promoting a healthy ocular surface and maintaining normal visual function. The main lacrimal gland comprises palpebral and orbital lobes, which are continuous with each other at the lateral edge of the aponeurosis of levator palpebrae superioris. The lobules have many acini and intralobular ducts that form excretory ducts that open into the fornix of the conjunctiva. The main lacrimal gland is comprised of acinar cells, ductal cells, and/or myoepithelial cells (Obata Cornea.; 25(10 Suppl 1):S82-9 (2006)). The main lacrimal gland secretes an aqueous layer of tear film as well as mucins onto the ocular surface of the eye of a subject (see, e.g., Paulsen, F., et al (2004) Cell and tissue research, 316(2), 167-177). As used herein, the term “lacrimal gland” refers to the main lacrimal gland, as well as the Wolfring's glands and the Krause's glands of a subject. The accessory glands, known as the Wolfring's glands and Krause's glands are located in the eyelid. In the upper eyelid there are about 2 to 5 Wolfring's glands and about forty Krause's glands. In the lower eyelid there are about 6 to 8 Krause's glands. Specific location and anatomy of the lacrimal functional unit is well-known (Conrady et al., J Ophthalmol. Article ID 7542929 (2016)).

### Expression Cassettes

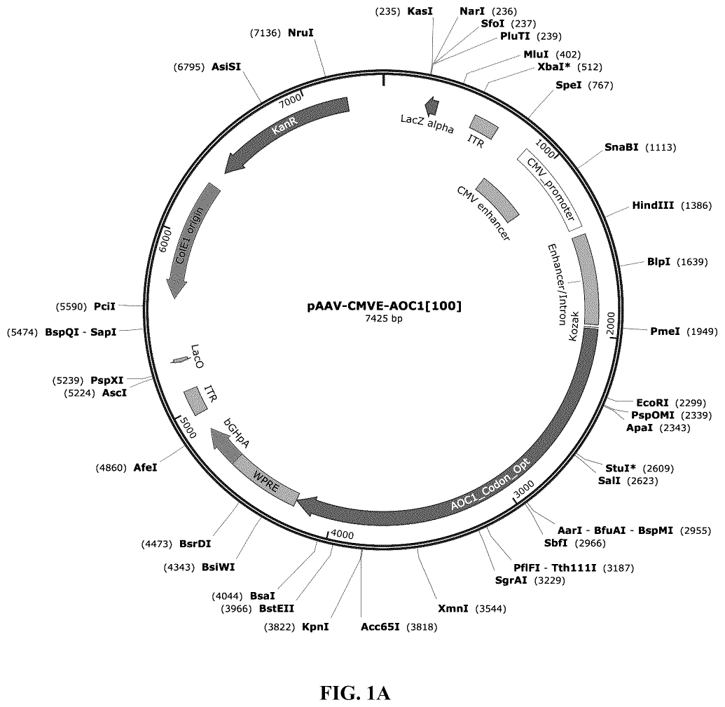

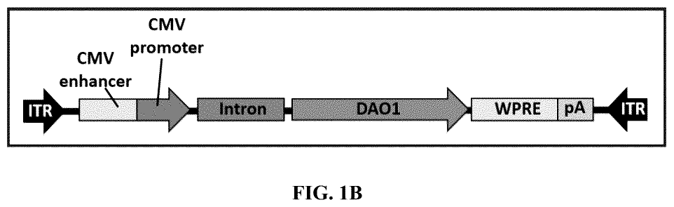

The rAAV vectors of the disclosure comprise an expression cassette. As used herein, the term “expression cassette” refers to a polynucleotide comprising at least one polynucleotide sequence encoding a protein of interest (e.g., DAO1) flanked by inverted terminal repeats. In some embodiments, the expression cassette further comprises other polynucleotide sequences, e.g., promoters, regulatory elements (e.g., one or more promoters), translation initiation sequences, coding sequences, and termination sequences (FIGS. 1A and 1B).

In mammals, histamine is metabolized by oxidative deamination via DAO1 (also called histaminase, amiloride-binding protein, amine oxidase copper containing 1, AOC1) (see, e.g., Elmore, et al (2002) J Biol Inorg Chem 7:565; Schwelberger, et al (2018) Inflamm Res 67:245; Finney et al (2014) Arch Biochem Biophys 546:19). The enzyme is a homodimer that catalyzes oxidative deamination of the primary amine group of histamine.

Histamine intolerance is the build-up of excessive levels of histamine due to decreased catabolism and/or increased production. Vakal et al., Molecules 2020, 25, 1293. Histamine is released during anaphylaxis and mast cell degranulation, and it has been suggested that mast cell degranulation increases DAO1 release and DAO1 is increased in severe anaphylaxis in mastocytosis patients (Boehm et al. Allergy. 2019, 74:58). Moreover, several therapeutic agents have been shown to inhibit DAO1, including, for example, berenil, pentamidine, aminoguanidine, metformin (Vakal 2020).

Excess histamine causes a range of symptoms (see, e.g., Schnedl et al., Food Sci Biotechnol (2019) 28(6):1779-1784). Histamine is a pro-inflammatory mediator contributing to allergic reactions (see, e.g., White MV (1990), J Allergy Clin Immunol 86: 599-605) and may contribute to Vernal Keratoconjunctivitis (VKC) (see, e.g., Abelson et al., Opthamology 1995, 102(12): 1958-1963; Bonini et al., (1992), Journal of allergy and clinical immunology, 89(1), 103-107).

Accordingly, the present disclosure provides expression cassettes comprising a polynucleotide sequence that encodes DAO1 for use in a therapy that induces breakdown of histamine (e.g., histamine present in the tear fluid). In some embodiments, the expression cassettes are used in a method of treating a condition (e.g., an ocular condition) associated with increased histamine production.

### DAO1 Enzyme

In some embodiments, the expression cassette of the present disclosure comprises a polynucleotide sequence encoding a DAO1 enzyme or a functional variant thereof. As used herein, the term “DAO1 enzyme” refers to a DAO1 enzyme from any species. The term “functional variant” refers to variants having sequence substitutions, insertions, deletions, and/or N- or C-terminal truncations, where the functional variant retains one or more functions of the reference protein, e.g., a native DAO1 enzyme. DAO1 is a 200-kDa homomeric glycoprotein that catalyzes oxidative deamination of the primary amino group of histamine.

In some embodiments, the DAO1 enzyme is a human DAO1 enzyme. In some embodiments, the human DAO1 enzyme is identified in a public database. Human DAO1 isoforms known in the art are typically identified via public databases. For example, the National Library of Medicine National Center for Biotechnology Information (NCBI) Gene Database (accessible via the world wide web: ncbi.nlm.nih.gov/) is a searchable database of genes that provides nomenclature, chromosomal localization, gene products, attributes of the gene, associated markers, phenotypes, interactions, links to citations, sequence information, information regarding sequence variants, gene maps, expression reports, homologs, protein domain content, and access to external databases. As is understood by the skilled artisan, sequence information for human DAO1 isoforms known in the art may be identified by entering the appropriate accession number into the NCBI Gene Database and selecting sequence information in the desired computer-readable format (e.g., FASTA). Such sequence information may include, but is not limited to, the nucleotide sequence for the full-length gene encoding DAO, the nucleotide sequence of the pre-mRNA transcript encoding DAO, the nucleotide sequence of the mRNA encoding DAO, the nucleotide sequence of the open reading frame (ORF) encoding DAO, and the amino acid sequence of DAO. For example, one isoform of human DAO1 is identifiable in the NCBI gene database via the accession number NM_001272072.2 and has the amino acid sequence set forth in SEQ ID NO: 2. In another example, one isoform of human DAO1 is identifiable in the NCBI gene database via the accession number NM_001091 and consists of the amino acid sequence set forth by SEQ ID NO: 2.

Exemplary amino acid sequences of human DAO1 and exemplary polynucleotide sequences (mRNA and ORF) encoding human DAO1 are set forth in Table 1. In some embodiments, the DAO1 enzyme comprises or consists of an amino acid sequence set forth in Table 1. In some embodiments, the DAO1 enzyme comprises or consists of an amino acid sequence encoded by an mRNA sequence set forth in Table 1. In some embodiments, the DAO1 enzyme comprises or consists of an amino acid sequence encoded by an ORF set forth in Table 1.

In some embodiments, the DAO1 enzyme comprises an amino acid sequence having at least 80%, at least 85%, at least 90%, at least 95%, at least 96%, at least 97%, at least 98%, or at least 99% identity to SEQ ID NO: 1. In some embodiments, the DAO1 enzyme comprises or consists of SEQ ID NO: 1. In some embodiments, the DAO1 enzyme comprises an amino acid sequence having at least 80%, at least 85%, at least 90%, at least 95%, at least 96%, at least 97%, at least 98%, or at least 99% identity to SEQ ID NO: 2. In some embodiments, the DAO1 enzyme comprises or consists of SEQ ID NO: 2.

In some embodiments, the DAO1 enzyme comprises an amino acid sequence having at least 80%, at least 85%, at least 90%, at least 95%, at least 96%, at least 97%, at least 98%, or at least 99% identity to one or more segments of SEQ ID NO: 1, including without limitation the segments spanning positions 39-125, 141-241, and/or 300-727 of SEQ ID NO: 1. In some embodiments, the DAO1 enzyme is encoded by an mRNA comprising or consisting of a nucleotide sequence that encodes one or more segments of SEQ ID NO: 1 (e.g., the segments spanning positions 39-125, 141-241, and/or 300-727 of SEQ ID NO: 1). In some embodiments, the DAO1 enzyme comprises an amino acid sequence having at least 80%, at least 85%, at least 90%, at least 95%, at least 96%, at least 97%, at least 98%, or at least 99% identity to one or more segments of SEQ ID NO: 2, including without limitation the segments spanning positions 39-125, 141-241, and/or 300-708 of SEQ ID NO: 2. In some embodiments, the DAO1 enzyme is encoded by an mRNA comprising or consisting of a nucleotide sequence that encodes one or more segments of SEQ ID NO: 2 (e.g., the segments spanning positions 39-125, 141-241, and/or 300-708 of SEQ ID NO: 2).

In some embodiments, the DAO1 enzyme comprises an amino acid sequence having at least 80%, at least 85%, at least 90%, at least 95%, at least 96%, at least 97%, at least 98%, or at least 99% identity to one or more segments of SEQ ID NO: 1 or SEQ ID NO: 2, wherein each segment comprises at least 20, 40, 60, 80, 100, 120, 140, 160, 180, 200, 220, 240, 260, 280, 300, 320, 340, 360, 380, 400, 420, 440, 460, 480, or 500 contiguous amino acids of SEQ ID NO: 1 or SEQ ID NO: 2, segment comprising a length within a range defined by any pair of the foregoing values. For example, a functional DAO1 enzyme may share high sequence identity (e.g., 95%) with one or more predicted functional domains (e.g., positions 39-125, 141-241, and/or 300-727 of SEQ ID NO: 1; or 39-125, 141-241, and/or 300-708 of SEQ ID NO: 2) while having greater sequence divergence in other portions of the DAO1 enzyme. Similarly, in some embodiments, a functional DAO1 enzyme may have at least 80%, at least 85%, at least 90%, at least 95%, at least 96%, at least 97%, at least 98%, or at least 99% sequence identity to a segment spanning from position 20 to the final position of either SEQ ID NO: 1 or SEQ ID NO: 2 (e.g., omitting a predicted N-terminal signal peptide shared by both sequences).

In some embodiments, the DAO1 enzyme is encoded by an mRNA comprising or consisting of a nucleotide sequence having at least 80%, at least 85%, at least 90%, at least 95%, at least 96%, at least 97%, at least 98%, or at least 99% identity to SEQ ID NO: 3. In some embodiments, the DAO1 enzyme is encoded by an mRNA comprising or consisting of SEQ ID NO: 3. In some embodiments, the DAO1 enzyme is encoded by an mRNA comprising or consisting of a nucleotide sequence having at least 80%, at least 85%, at least 90%, at least 95%, at least 96%, at least 97%, at least 98%, or at least 99% identity to SEQ ID NO: 4. In some embodiments, the DAO1 enzyme is encoded by an mRNA comprising or consisting of SEQ ID NO: 4.

In some embodiments, the DAO1 enzyme is encoded by an ORF comprising or consisting of a nucleotide sequence having at least 80%, at least 85%, at least 90%, at least 95%, at least 96%, at least 97%, at least 98%, or at least 99% identity to SEQ ID NO: 29. In some embodiments, the DAO1 enzyme is encoded by an ORF comprising or consisting of SEQ ID NO: 29. In some embodiments, the DAO1 enzyme is encoded by an ORF comprising or consisting of a nucleotide sequence having at least 80%, at least 85%, at least 90%, at least 95%, at least 96%, at least 97%, at least 98%, or at least 99% identity to SEQ ID NO: 30. In some embodiments, the DAO1 enzyme is encoded by an ORF comprising or consisting of SEQ ID NO: 30. In some embodiments, the DAO1 enzyme is encoded by an ORF comprising or consisting of a nucleotide sequence having at least 80%, at least 85%, at least 90%, at least 95%, at least 96%, at least 97%, at least 98%, or at least 99% identity to SEQ ID NO: 31. In some embodiments, the DAO1 enzyme is encoded by an ORF comprising or consisting of SEQ ID NO: 31.

### Polynucleotides Encoding a DAO1 Enzyme

In some embodiments, the expression cassette comprises a polynucleotide encoding a DAO1 enzyme described herein (e.g., a human DAO1 enzyme). In some embodiments, the polynucleotide comprises an ORF encoding a DAO1 enzyme described herein (e.g., a human DAO1 enzyme).

In some embodiments, the expression cassette provides increased expression of DAO1 in the at least one eye and/or lacrimal gland. In some embodiments, expression of DAO1 may be increased 5%, 10%, 15%, 20%, or 25% compared to expression of DAO1 in an untreated subject, or in the contralateral eye of a treated subject. As used herein, “subject” means any mammal, including mice, rabbits, non-human primates (NIP) and humans. In some embodiments, the subject is a human or NHP. Moreover, “individual” or “patient” may be used interchangeably with “subject.” In some embodiments, expression of DAO1 may be increased by at least 1.5-fold, 2-fold, 3-fold, 4-fold, or 5-fold compared to expression of DAO1 in an untreated subject, or in the contralateral eye of a treated subject. In some embodiments, DAO1 may be expression at any detectable level in the treated eye, whereas DAO1 may be not be expressed, or expressed at undetectable levels, in an untreated subject, or in the contralateral eye of a treated subject. Put another way, the eye or lacrimal gland to which an rAAV vector described herein is administered may express DAO1 in higher abundance than in an eye or lacrimal gland that has only endogenous (i.e., native) expression of DAO1 or in eyes that have a lower or impaired secretion of endogenous (i.e., native) DAO1.

The term “functional variant” refers to variants having sequence substitutions, insertions, deletions, and/or N- or C-terminal truncations, where the functional variant retains one or more functions of the reference protein, e.g., native DAO1.

In some embodiments, the expression cassette comprises a polynucleotide encoding a protein that is human DAO1 or a functional variant thereof. In some embodiments, the expression cassette comprises a polynucleotide comprising a nucleotide sequence encoding an amino acid sequence identified in Table 1. In some embodiments, the expression cassette comprises a polynucleotide comprising a nucleotide sequence identified in Table 1.

In some embodiments, the expression cassette comprises a polynucleotide comprising a nucleotide sequence encoding a protein having at least 80%, at least 85%, at least 90%, at least 95%, at least 96%, at least 97%, at least 98%, or at least 99% identity to the amino acid sequence set forth in SEQ ID NO: 1. In some embodiments, the expression cassette comprises a polynucleotide comprising a nucleotide sequence encoding the amino acid sequence set forth in SEQ ID NO: 1. In some embodiments, the expression cassette comprises a polynucleotide comprising a nucleotide sequence encoding a protein having at least 80%, at least 85%, at least 90%, at least 95%, at least 96%, at least 97%, at least 98%, or at least 99% identity with SEQ ID NO: 2. In some embodiments, the expression cassette comprises a polynucleotide comprising a nucleotide sequence encoding the amino acid sequence set forth in SEQ ID NO: 2.

In some embodiments, the expression cassette comprises a polynucleotide comprising a nucleotide sequence encoding a DAO1 enzyme, wherein the DAO1 enzyme comprises or consists of an amino acid sequence having at least 80%, at least 85%, at least 90%, at least 95%, at least 96%, at least 97%, at least 98%, or at least 99% identity to SEQ ID NO: 1. In some embodiments, the DAO1 enzyme comprises or consists of SEQ ID NO: 1. In some embodiments, the expression cassette comprises a polynucleotide comprising a nucleotide sequence encoding a DAO1 enzyme, wherein the DAO1 enzyme comprises or consists of an amino acid sequence having at least 80%, at least 85%, at least 90%, at least 95%, at least 96%, at least 97%, at least 98%, or at least 99% identity to SEQ ID NO: 2. In some embodiments, the DAO1 enzyme comprises or consists of SEQ ID NO: 2.

In some embodiments, the expression cassette comprises a polynucleotide encoding DAO1 comprising a nucleotide sequence that shares at least 80%, at least 85%, at least 90%, at least 95%, at least 96%, at least 97%, at least 98%, or at least 99% identity with SEQ ID NO: 30.

In some embodiments, the expression cassette comprises a polynucleotide encoding DAO1 comprising or consisting of the nucleotide sequence set forth in SEQ ID NO: 30. In some embodiments, the expression cassette comprises a polynucleotide encoding DAO1 comprising or consisting of a nucleotide sequence that shares at least 80%, at least 85%, at least 90%, at least 95%, at least 96%, at least 97%, at least 98%, or at least 99% identity with SEQ ID NO: 31. In some embodiments, the expression cassette comprises a polynucleotide comprising or consisting of the nucleotide sequence set forth in SEQ ID NO: 31. In some embodiments, the protein is human DAO1 or a functional variant thereof.

### Codon Optimization

In some embodiments, the expression cassette comprises a polynucleotide encoding a DAO1 enzyme, wherein the polynucleotide comprises or consists of a nucleotide sequence codon-optimized for expression in a target cell. In some embodiments, the target cell is a mammalian cell. In some embodiments, the target cell is a human cell, a murine cell, or a non-human primate (NHP) cell.

A codon-optimized nucleotide sequence, e.g., a codon-optimized nucleotide sequence encoding a DAO1 enzyme, typically is a sequence comprising at least one synonymous nucleobase substitution with respect to a reference sequence (e.g., a wild-type ORF encoding a DAO1 enzyme). A codon-optimized nucleotide sequence can be partially or completely different in sequence from the reference sequence. For example, a reference sequence encoding polyserine uniformly encoded by TCT codons can be sequence-optimized by having 100% of its nucleobases substituted (for each codon, T in position 1 replaced by A, C in position 2 replaced by G, and T in position 3 replaced by C) to yield a sequence encoding polyserine which would be uniformly encoded by AGC codons. The percentage of sequence identity obtained from a global pairwise alignment between the reference polyserine nucleic acid sequence and the sequence-optimized polyserine nucleic acid sequence would be 0%. However, the protein products from both sequences would be 100% identical.

Codon optimization methods are known in the art and can be useful to achieve one or more desired results, e.g., to increase expression of a synthetic gene in a target cell. In some embodiments, an expression cassette comprises a nucleotide sequence that is sequence-optimized relative to a reference sequence using a method of sequence optimization. Methods of sequence optimization are known in the art, and include known sequence optimization tools, algorithms and services. Non-limiting examples include services from GeneArt (Life Technologies), DNA2.0 (Menlo Park CA), Geneious®, and GeneGPS® (Atum, Newark, CA).

In some embodiments, an expression cassette of the disclosure comprises a polynucleotide encoding a DAO1 enzyme that is sequence-optimized relative to a reference sequence using a method of sequence optimization (e.g., GeneGPS®, e.g., Geneious®). In some embodiments, the method of sequence optimization comprises a codon optimization algorithm described in U.S. Pat. Nos. 7,561,972; 7,561,973; 8,126,653; and 8,401,798, each of which is incorporated herein by reference. In some embodiments, the polynucleotide sequence is sequence-optimized based on codon usage bias in a host cell (e.g., mammalian cell, e.g., human cell, murine cell, non-human primate cell) relative to a reference sequence, using a method of sequence optimization known in the art (e.g., GeneGPS®, e.g., Geneious®). In some embodiments, the polynucleotide sequence comprises or consists of a nucleotide sequence that is codon-optimized relative to a reference sequence using a method of sequence optimization, wherein the reference sequence is selected from SEQ ID NOs: 30 and 31. In some embodiments, the polynucleotide sequence comprises or consists of a nucleotide sequence that is codon-optimized relative to a reference sequence for expression in a human host cell, wherein the reference sequence is selected from SEQ ID NOs: 30 and 31. In some embodiments, the polynucleotide sequence comprises or consists of SEQ ID NO: 29.

In some embodiments, the disclosure provides an expression cassette comprising a polynucleotide comprising or consisting of a nucleotide sequence having at least 80%, at least 85%, at least 90%, at least 95%, at least 96%, at least 97%, at least 98%, or at least 99% identity to SEQ ID NO: 29. In some embodiments, the polynucleotide comprises or consists of SEQ ID NO: 29.

TABLE 1
Human DAO1 Amino Acid and Polynucleotide Sequences

| Name/ | NCBI Accession | Amino acid | mRNA | ORF Nucleotide |
|---|---|---|---|---|
| identifier | No. | sequence | sequence | sequence |
| AOC1 variant 1 | NM_001272072.2 | SEQ ID NO: 1 | SEQ ID NO: 3 | SEQ ID NO: 30 |
| AOC1 variant 2 | NM_001091.4 | SEQ ID NO: 2 | SEQ ID NO: 4 | SEQ ID NO: 31 |
| AOC1 variant 2 | — | SEQ ID NO: 2 | — | SEQ ID NO: 29 |
| (codon optimized) | | | | |

### Expression Cassette Elements

In some embodiments, the expression cassette of the present disclosure comprises a promoter. The term “promoter” as used herein refers to a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific, tissue-specific, or species specific. Promoters may be “constitutive,” meaning continually active, or “inducible,” meaning the promoter can be activated or deactivated by the presence or absence of biotic or abiotic factors. Also included in the nucleic acid constructs or vectors of the invention are enhancer sequences that may or may not be contiguous with the promoter sequence. Enhancer sequences influence promoter-dependent gene expression and may be located in the 5′ or 3′ regions of the native gene.

Any suitable promoter region or promoter sequence therein can be used in the subject polynucleotide cassettes, so long as the promoter region promotes expression of a polynucleotide sequence encoding a DAO1 enzyme in the at least one eye and/or lacrimal glands. In some embodiments, the promoter promotes expression of the gene in mammalian eye and/or lacrimal glands. In some embodiments, the expression cassette comprises a cell-type specific promoter. The promoter may specifically promote transcription in the cells of the eye and/or the cells of the lacrimal gland.

In some embodiments, the promoter is a CAG promoter. In some embodiments, the promoter comprises a nucleotide sequence having at least 90%, 95%, 96%, 97%, 98%, or 99% identity with the nucleotide sequence set forth in SEQ ID NO: 5. In some embodiments, the promoter comprises SEQ ID NO: 5.

In some embodiments, the promoter is a CMV promoter. In some embodiments, the promoter comprises a nucleotide sequence having at least 95%, 96%, 97%, 98%, or 99% identity to SEQ ID NO: 21. In some embodiments, the promoter comprises or consists of SEQ ID NO: 21.

The expression cassette may contain a polyadenylation (polyA) sequence. In some embodiments, the expression cassettes described herein comprise a transcription termination signal. Elements directing the efficient termination and polyadenylation of the heterologous nucleic acid transcripts increases heterologous gene expression. Transcription termination signals are generally found downstream of the polyadenylation signal. In some embodiments, vectors comprise a polyadenylation sequence 3′ of a polynucleotide encoding a polypeptide to be expressed. The term “polyA site” or “polyA sequence” as used herein denotes a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Cleavage and polyadenylation is directed by a poly(A) sequence in the RNA. The core poly(A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly(A) sequence is an ideal polyA sequence (e.g., AATAAA, ATTAAA, AGTAAA). In particular embodiments, the poly(A) sequence is an SV40 polyA sequence, a bovine growth hormone polyA sequence (BGH polyA), a rabbit β-globin polyA sequence (rpgpA), variants thereof, or another suitable heterologous or endogenous polyA sequence known in the art. In some embodiments, the expression cassette described herein comprises a polyA sequence. In some embodiments, the polyA sequence is a BGH polyA sequence. In some embodiments, the BGH polyA sequence comprises or consists of a nucleotide sequence having at least 95%, 96%, 97%, 98%, or 99% identity with SEQ ID NO: 18. In some embodiments, the BGH polyA sequence comprises or consists of SEQ ID NO: 18.

Optionally the rAAV vectors of the disclosure comprise the Woodchuck Post-transcriptional Regulatory Element (WPRE). In some embodiments, the rAAV vector comprises a WPRE comprising SEQ ID NO: 22.

### Recombinant AAV Vector

In some embodiments of the present disclosure, the subject expression cassettes are used to deliver DAO1 or a functional variant thereof to at least one eye and/or lacrimal gland of a subject, e.g., to treat an ocular disorder. Accordingly, in some embodiments of the disclosure, the composition that provides for the expression of DAO1 or a functional variant thereof in at least one eye and/or lacrimal gland of a subject is a gene delivery vector, wherein the gene delivery vector comprises an expression cassette described herein.

In some embodiments, the gene delivery vector is an rAAV vector. In such embodiments, the expression cassette is flanked on the 5′ and 3′ ends by functional AAV inverted terminal repeat (ITR) sequences. By “functional AAV ITR sequences” is meant that the ITR sequences function as intended for the rescue, replication and packaging of the AAV vector. Hence, AAV ITRs for use in the gene delivery vectors of the present disclosure need not have a wild-type nucleotide sequence, and may be altered by the insertion, deletion or substitution of nucleotides or the AAV ITRs may be derived from any of several AAV serotypes, e.g., AAV1, AAV2, AAV3, AAV4, AAV5, AAV6, AAV7, AAV8, AAV9, AAV10. In some embodiments, the AAV ITR is derived from AAV1. In some embodiments, the AAV ITR is derived from AAV2. In some embodiments, the AAV ITR is derived from AAV3. In some embodiments, the AAV ITR is derived from AAV4. In some embodiments, the AAV ITR is derived from AAV5. In some embodiments, the AAV ITR is derived from AAV6. In some embodiments, the AAV ITR is derived from AAV7. In some embodiments, the AAV ITR is derived from AAV9. In some embodiments, the AAV ITR is derived from AAV1. In some embodiments, the AAV ITR is derived from AAV10. Certain rAAV vectors have the wild-type REP and CAP genes deleted in whole or part, but retain functional flanking ITR sequences. In some embodiments, the AAV ITR is one identified in Table 2.

TABLE 2
Exemplary ITR Sequences

| | | SEQ |
|---|---|---|
| | | ID |
| Name | Nucleotide Sequence | NO |
| 5′ | CCTGCAGGCAGCTGCGCGCTCGCTCGCTCACTGAGGCCG | 19 |
| ITR | CCCGGGCAAAGCCCGGGCGTCGGGCGACCTTTGGTCGCC | |
| | CGGCCTCAGTGAGCGAGCGAGCGCGCAGAGAGGGAGTG | |
| | GCCAACTCCATCACTAGGGGTTCCT | |
| 5′ | AGGAACCCCTAGTGATGGAGTTGGCCACTCCCTCTCTGC | 20 |
| ITR | GCGCTCGCTCGCTCACTGAGGCCGGGCGACCAAAGGTCG | |
| | CCCGACGCCCGGGCTTTGCCCGGGCGGCCTCAGTGAGCG | |
| | AGCGAGCGCGCAGCTGCCTGCAGG | |
| AAV2 | GCGCGCTCGCTCGCTCACTGAGGCCGCCCGGGCAAAGCC | 16 |
| 5′ | CGGGCGTCGGGCGACCTTTGGTCGCCCGGCCTCAGTGAG | |
| ITR | CGAGCGAGCGCGCAGAGAGGGAGTGGCCAACTCCATCA | |
| | CTAGGGGTTCCTTGTAGTTAATGATTAAC | |
| AAV2 | GTTAATCATTAACTACAAGGAACCCCTAGTGATGGAGTT | 17 |
| 3′ | GGCCACTCCCTCTCTGCGCGCTCGCTCGCTCACTGAGGC | |
| ITR | CGGGCGACCAAAGGTCGCCCGACGCCCGGGCTTTGCCCG | |
| | GGCGGCCTCAGTGAGCGAGCGAGCGCGC | |

In such embodiments, the rAAV vector comprises an AAV capsid, derived from any adeno-associated virus serotype known in the art, or prospectively discovered, including without limitation, AAV1, AAV2, AAV3, AAV4, AAV5, AAV6, AAV7, AAV8, AAV9, AAV10, etc. For example, the AAV capsid may be a wild-type (or “native”) capsid. In some embodiments, the rAAV vector comprises an AAV capsid derived from AAV1. In some embodiments, the rAAV vector comprises an AAV capsid derived from AAV2. In some embodiments, the rAAV vector comprises an AAV capsid derived from AAV3. In some embodiments, the rAAV vector comprises an AAV capsid derived from AAV4. In some embodiments, the rAAV vector comprises an AAV capsid derived from AAV5. In some embodiments, the rAAV vector comprises an AAV capsid derived from AAV6. In some embodiments, the rAAV vector comprises an AAV capsid derived from AAV7. In some embodiments, the rAAV vector comprises an AAV capsid derived from AAV8. In some embodiments, the rAAV vector comprises an AAV capsid derived from AAV9. In some embodiments, the rAAV vector comprises an AAV capsid derived from AAV10. AAV capsids of particular interest include AAV2, AAV5, AAV8, and AAV9. However, as with the ITRs, the capsid need not have a wild-type nucleotide sequence, but rather may be altered by the insertion, deletion or substitution of nucleotides in the VP1, VP2 or VP3 sequence, so long as the capsid is able to transduce cells of the eye and/or lacrimal glands. Put another way, the AAV capsid may be a variant AAV capsid. In some embodiments, the rAAV vector is a “pseudotyped” AAV created by using the capsid (cap) gene of one AAV and the rep gene and ITRs from a different AAV, e.g., a pseudotyped AAV2 created by using rep from AAV2 and cap from AAV1, AAV3, AAV4, AAV5, AAV6, AAV7, AAV8, or AAV9 together with a plasmid containing a vector based on AAV2. For example, the rAAV vector may be rAAV2/1, rAAV2/3, rAAV2/4, rAAV2/5, rAAV2/6, rAAV2/7, rAAV2/8, rAAV2/9, etc. In some embodiments, the rAAV is rAAV2/1. In some embodiments, the rAAV is rAAV2/3. In some embodiments, the rAAV is rAAV2/4. In some embodiments, the rAAV is rAAV2/5. In some embodiments, the rAAV is rAAV2/6. In some embodiments, the rAAV is rAAV2/7. In some embodiments, the rAAV is rAAV2/8. In some embodiments, the rAAV is rAAV2/9.

In some embodiments, the rAAV is replication defective, in that the rAAV vector cannot independently further replicate and package its genome. For example, when eye and/or lacrimal glands are transduced with rAAV vectors, the gene is expressed in the transduced eye and/or lacrimal gland, however, due to the fact that the transduced eye and/or lacrimal glands lack AAV rep and cap genes and accessory function genes, the rAAV is not able to replicate.

rAAV vectors of the present disclosure encapsulating the expression cassettes as described herein, can be produced using helper-free production. rAAVs are replication-deficient viruses and normally require components from a live helper virus, such as adenovirus, in a host cell for packaging of infectious rAAV vectors. rAAV helper-free production systems allow the production of infectious rAAV vectors without the use of a live helper virus. In the helper-free system, a host packaging cell line is co-transfected with three plasmids. A first plasmid contains adenovirus gene products (i.e. E2A, E4, and VA RNA genes) needed for the packaging of rAAV vectors. A second plasmid contains the required AAV genes (i.e., REP and CAP genes). A third plasmid contains the polynucleotide sequence encoding DAO1 or a functional variant thereof and a promoter flanked by ITRs. A host packaging cell line can be, for example, AAV-293 host cells. Suitable host cells contain additional components required for packaging infectious rAAV vectors that are not supplied by the plasmids. In some embodiments, the CAP genes can encode, for example, AAV capsid proteins as described herein. In some embodiments, the promoter is a promoter sequence as described herein. In some embodiments, the promoter sequence is a CAG sequence. In some embodiments, the encoded enzyme is DAO1.

AAV serotypes shown to infect the eye and/or lacrimal gland include AAV2, AAV5, AAV 5w8, and AAV9 (Rocha et al., supra). Exemplary amino acid and nucleotide sequences for the AAV capsid proteins are identified in Table 3.

In some embodiments, the AAV serotype used to infect the eye and/or lacrimal gland is AAV2. In some embodiments, the AAV capsid protein comprises SEQ ID NO: 6. In some embodiments, the AAV capsid protein comprises a sequence at least 95%, 96%, 97%, 98%, 99%, or 100% similar to SEQ ID NO: 6. In some embodiments, the AAV capsid protein shares at least 95%, 98%, or 100% identity to the AAV2 VP1 protein (SEQ ID NO: 6).

In some embodiments, the polynucleotide sequence encoding the AAV2 VP1 protein comprises SEQ ID NO: 7. In some embodiments, the polynucleotide sequence encoding the AAV2 VP1 protein comprises a sequence at least 95%, 96%, 97%, 98%, 99%, or 100% similar to SEQ ID NO: 7. In some embodiments, the polynucleotide sequence encoding the AAV2 VP1 protein shares at least 95%, 98%, or 100% identity with SEQ ID NO: 7.

In some embodiments, the AAV capsid protein comprises SEQ ID NO: 8. In some embodiments, the AAV capsid protein comprises a sequence at least 95%, 96%, 97%, 98%, 99%, or 100% similar to SEQ ID NO: 8. In some embodiments, the AAV capsid protein shares at least 95%, 98%, or 100% identity to the AAV2 VP3 protein (SEQ ID NO: 8).

In some embodiments, the polynucleotide sequence encoding the AAV2 VP3 protein comprises SEQ ID NO: 9. In some embodiments, the polynucleotide sequence encoding the AAV capsid protein comprises a sequence at least 95%, 96%, 97%, 98%, 99%, or 100% similar to SEQ ID NO: 9. In some embodiments, the polynucleotide sequence encoding the AAV2 VP3 protein shares at least 95%, 98%, or 100% identity with SEQ ID NO: 9.

In some embodiments, the AAV serotype used to infect the eye and/or lacrimal gland is AAV5. In some embodiments, the AAV capsid protein comprises SEQ ID NO: 10. In some embodiments, the AAV capsid protein comprises a sequence at least 95%, 96%, 97%, 98%, 99%, or 100% similar to SEQ ID NO: 10. In some embodiments, the AAV capsid protein shares at least 95%, 98%, or 100% identity to the AAV5 capsid protein (SEQ ID NO: 10).

In some embodiments, the polynucleotide sequence encoding the AAV5 capsid protein comprises SEQ ID NO: 11. In some embodiments, the polynucleotide sequence encoding the AAV capsid protein comprises a sequence at least 95%, 96%, 97%, 98%, 99%, or 100% similar to SEQ ID NO: 11. In some embodiments, the polynucleotide sequence encoding the AAV5 capsid protein shares at least 95%, 98%, or 100% identity with SEQ ID NO: 11.

In some embodiments, the AAV serotype used to infect the eye and/or lacrimal gland is AAV8. In some embodiments, the capsid protein comprises SEQ ID NO: 12. In some embodiments, the AAV capsid protein comprises a sequence at least 95%, 96%, 97%, 98%, 99%, or 100% similar to SEQ ID NO: 12. In some embodiments, the AAV capsid protein shares at least 95%, 98%, or 100% identity to the AAV8 capsid protein (SEQ ID NO: 12).

In some embodiments, the polynucleotide encoding the AAV8 capsid protein comprises SEQ ID NO: 13. In some embodiments, the polynucleotide sequence encoding the AAV capsid protein comprises a sequence at least 95%, 96%, 97%, 98%, 99%, or 100% similar to SEQ ID NO: 13. In some embodiments, the polynucleotide sequence encoding the AAV8 capsid protein shares at least 95%, 98%, or 100% identity with SEQ ID NO: 13.

In some embodiments, the AAV serotype used to infect the eye and/or lacrimal gland is AAV9. In some embodiments, the AAV capsid protein comprises SEQ ID NO: 14. In some embodiments, the AAV capsid protein comprises a sequence at least 95%, 96%, 97%, 98%, 99%, or 100% similar to SEQ ID NO: 14. In some embodiments, the AAV capsid protein shares at least 95%, 98%, or 100% identity to the AAV9 capsid protein (SEQ ID NO: 14).

In some embodiments, the polynucleotide sequence encoding the AAV9 capsid protein comprises SEQ ID NO: 15. In some embodiments, the polynucleotide sequence encoding the AAV capsid protein comprises a sequence at least 95%, 96%, 97%, 98%, 99%, or 100% similar to SEQ ID NO: 15. In some embodiments, the polynucleotide sequence encoding the AAV9 capsid protein shares at least 95%, 98%, or 100% identity with SEQ ID NO: 15.

TABLE 3
AAV Capsid Sequences

| | | Nucleotide (Open |
|---|---|---|
| | Protein | Reading Frame) |
| AAV2 VP1 | SEQ ID NO: 6 | SEQ ID NO: 7 |
| AAV2 VP3 | SEQ ID NO: 8 | SEQ ID NO: 9 |
| AAV5 | SEQ ID NO: 10 | SEQ ID NO: 11 |
| AAV8 | SEQ ID NO: 12 | SEQ ID NO: 13 |
| AAV9 | SEQ ID NO: 14 | SEQ ID NO: 15 |

Exemplary rAAVs

In some embodiments, the rAAV comprises an AAV capsid. In some embodiments, an rAAV described herein comprises an expression cassette comprising a polynucleotide comprising a nucleotide sequence encoding DAO1. In some embodiments, the polynucleotide is operably linked to a promoter. In some embodiments, the rAAV comprises an AAV capsid and the expression cassette, wherein the expression cassette comprises a polynucleotide operatively linked to a promoter, wherein the polynucleotide comprises a nucleotide sequence encoding DAO1. In some embodiments, the promoter is any described herein or known in the art. In some embodiments, the promoter is a CAG promoter. In some embodiments, the promoter is a CMV promoter. In some embodiments, the expression cassette further comprises a 5′ITR and/or a 3′ITR. In some embodiments, the 5′ITR is an AAV2 5′ITR. In some embodiments, the 3′ITR is a AAV2 3′ITR. In some embodiments, the expression cassette comprises a nucleotide sequence comprising from 5′ to 3′: a 5′ITR (e.g., an AAV2 5′ITR), a promoter (e.g., a CMV promoter), a 5′untranslated region (5′UTR), a polynucleotide sequence comprising a nucleotide sequence encoding a DAO1 enzyme, a polyA sequence, and a 3′ITR (e.g., an AAV2 3′ITR). In some embodiments, the expression cassette comprises a nucleotide sequence comprising from 5′ to 3′: a 5′ITR (e.g., an AAV2 5′ITR), an enhancer (e.g., a CMV enhancer), a promoter (e.g., a CMV promoter), a 5′UTR, a polynucleotide sequence comprising a nucleotide sequence encoding a DAO1 enzyme, a WPRE sequence, a polyA sequence, and a 3′ITR (e.g., an AAV2 3′ITR). In some embodiments, the 5′UTR comprises a Kozak sequence positioned immediately upstream of and adjacent to an initiation codon in the nucleotide sequence encoding the DAO1 enzyme.

In some embodiments, the rAAV comprises an AAV capsid and an expression cassette comprising a polynucleotide operatively linked to a promoter, wherein the polynucleotide comprises a nucleotide sequence encoding a human DAO1 enzyme described herein. In some embodiments, the rAAV comprises an AAV capsid and an expression cassette comprising a polynucleotide operatively linked to a CAG promoter or a CMV promoter, wherein the polynucleotide comprises a nucleotide sequence encoding a DAO1 enzyme described herein.

In some embodiments, the rAAV comprises an AAV capsid and an expression cassette comprising a polynucleotide operatively linked to a promoter (e.g., a CAG promoter or CMV promoter), wherein the polynucleotide comprises a nucleotide sequence encoding a DAO1 enzyme, wherein the DAO1 enzyme comprises or consists of an amino acid sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 1. In some embodiments, the DAO1 enzyme comprises or consists of the amino acid sequence set forth in SEQ ID NO: 1. In some embodiments, the rAAV comprises an AAV capsid and an expression cassette comprising a polynucleotide operatively linked to a promoter (e.g., a CAG promoter), wherein the polynucleotide comprises a nucleotide sequence encoding a DAO1 enzyme, wherein the nucleotide sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 30. In some embodiments, the nucleotide sequence comprises SEQ ID NO: 30.

In some embodiments, the rAAV comprises an AAV capsid and an expression cassette comprising a polynucleotide operatively linked to a promoter (e.g., a CAG promoter or CMV promoter), wherein the polynucleotide comprises a nucleotide sequence encoding a DAO1 enzyme, wherein the DAO1 enzyme comprises or consists of an amino acid sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 2. In some embodiments, the DAO1 enzyme comprises or consists of the amino acid sequence set forth in SEQ ID NO: 2. In some embodiments, the rAAV comprises an AAV capsid and an expression cassette comprising a polynucleotide operatively linked to a promoter (e.g., a CAG promoter or CMV promoter), wherein the polynucleotide comprises a nucleotide encoding a DAO1 enzyme, wherein the nucleotide sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 31. In some embodiments, the nucleotide sequence comprises SEQ ID NO: 31. In some embodiments, the rAAV comprises an AAV capsid and an expression cassette comprising a polynucleotide operatively linked to a promoter (e.g., a CAG promoter or CMV promoter), wherein the polynucleotide comprises a nucleotide encoding a DAO1 enzyme, wherein the nucleotide sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 29. In some embodiments, the nucleotide sequence comprises SEQ ID NO: 29.

In some embodiments, the rAAV comprises an AAV capsid and an expression cassette comprising or consisting of a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 23. In some embodiments, the expression cassette comprises or consists of SEQ ID NO: 23.

In some embodiments, the rAAV comprises an AAV2 capsid and an expression cassette comprising a polynucleotide operatively linked to a promoter, wherein the polynucleotide comprises a nucleotide sequence encoding a human DAO1 enzyme described herein. In some embodiments, the rAAV comprises an AAV2 capsid and an expression cassette comprising a polynucleotide operatively linked to a CAG promoter or a CMV promoter, wherein the polynucleotide comprises a nucleotide sequence encoding a DAO1 enzyme described herein.

In some embodiments, the rAAV comprises an AAV2 capsid and an expression cassette comprising a polynucleotide operatively linked to a promoter (e.g., a CAG promoter or CMV promoter), wherein the polynucleotide comprises a nucleotide sequence encoding a DAO1 enzyme, wherein the DAO1 enzyme comprises or consists of an amino acid sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 1. In some embodiments, the DAO1 enzyme comprises or consists of the amino acid sequence set forth in SEQ ID NO: 1. In some embodiments, the rAAV comprises an AAV2 capsid and an expression cassette comprising a polynucleotide operatively linked to a promoter (e.g., a CAG promoter or CMV promoter), wherein the polynucleotide comprises a nucleotide encoding a DAO1 enzyme, wherein the nucleotide sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 30. In some embodiments, the nucleotide sequence comprises SEQ ID NO: 30.

In some embodiments, the rAAV comprises an AAV2 capsid and an expression cassette comprising a polynucleotide operatively linked to a promoter (e.g., a CAG promoter or CMV promoter), wherein the polynucleotide comprises a nucleotide sequence encoding a DAO1 enzyme, wherein the DAO1 enzyme comprises or consists of an amino acid sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 2. In some embodiments, the DAO1 enzyme comprises the amino acid sequence set forth in SEQ ID NO: 2. In some embodiments, the rAAV comprises an AAV2 capsid and an expression cassette comprising a polynucleotide operatively linked to a promoter (e.g., a CAG promoter or CMV promoter), wherein the polynucleotide comprises a nucleotide encoding a DAO1 enzyme, wherein the nucleotide sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 31. In some embodiments, the nucleotide sequence comprises SEQ ID NO: 31. In some embodiments, the rAAV comprises an AAV2 capsid and an expression cassette comprising a polynucleotide operatively linked to a promoter (e.g., a CAG promoter or CMV promoter), wherein the polynucleotide comprises a nucleotide encoding a DAO1 enzyme, wherein the nucleotide sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 29. In some embodiments, the nucleotide sequence comprises SEQ ID NO: 29.

In some embodiments, the rAAV comprises an AAV2 capsid and an expression cassette comprising or consisting of a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 23. In some embodiments, the rAAV comprises an AAV2 capsid and an expression cassette comprising or consisting of SEQ ID NO: 23.

In some embodiments, the rAAV comprises an AAV5 capsid and an expression cassette comprising a polynucleotide operatively linked to a promoter, wherein the polynucleotide comprises a nucleotide sequence encoding a human DAO1 enzyme described herein. In some embodiments, the rAAV comprises an AAV5 capsid and an expression cassette comprising a polynucleotide operatively linked to a CAG promoter, wherein the polynucleotide comprises a nucleotide sequence encoding a DAO1 enzyme described herein.

In some embodiments, the rAAV comprises an AAV5 capsid and an expression cassette comprising a polynucleotide operatively linked to a promoter (e.g., a CAG promoter or CMV promoter), wherein the polynucleotide comprises a nucleotide sequence encoding a DAO1 enzyme, wherein the DAO1 enzyme comprises or consists of an amino acid sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 1. In some embodiments, the DAO1 enzyme comprises the amino acid sequence set forth in SEQ ID NO: 1. In some embodiments, the rAAV comprises an AAV5 capsid and an expression cassette comprising a polynucleotide operatively linked to a promoter (e.g., a CAG promoter or CMV promoter), wherein the polynucleotide comprises a nucleotide encoding a DAO1 enzyme, wherein the nucleotide sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 30. In some embodiments, the nucleotide sequence comprises SEQ ID NO: 30.

In some embodiments, the rAAV comprises an AAV5 capsid and an expression cassette comprising a polynucleotide operatively linked to a promoter (e.g., a CAG promoter or CMV promoter), wherein the polynucleotide comprises a nucleotide sequence encoding a DAO1 enzyme, wherein the DAO1 enzyme comprises an amino acid sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 2. In some embodiments, the DAO1 enzyme comprises the amino acid sequence set forth in SEQ ID NO: 2. In some embodiments, the rAAV comprises an AAV5 capsid and an expression cassette comprising a polynucleotide operatively linked to a promoter (e.g., a CAG promoter or CMV promoter), wherein the polynucleotide comprises a nucleotide encoding a DAO1 enzyme, wherein the nucleotide sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 31. In some embodiments, the nucleotide sequence comprises SEQ ID NO: 31. In some embodiments, the rAAV comprises an AAV5 capsid and an expression cassette comprising a polynucleotide operatively linked to a promoter (e.g., a CAG promoter or CMV promoter), wherein the polynucleotide comprises a nucleotide encoding a DAO1 enzyme, wherein the nucleotide sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 29. In some embodiments, the nucleotide sequence comprises SEQ ID NO: 29.

In some embodiments, the rAAV comprises an AAV5 capsid and an expression cassette comprising or consisting of a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 23. In some embodiments, the rAAV comprises an AAV5 capsid and an expression cassette comprising or consisting of SEQ ID NO: 23

In some embodiments, the rAAV comprises an AAV9 capsid and an expression cassette comprising a polynucleotide operatively linked to a promoter, wherein the polynucleotide comprises a nucleotide sequence encoding a human DAO1 enzyme described herein. In some embodiments, the rAAV comprises an AAV9 capsid and an expression cassette comprising a polynucleotide operatively linked to a CAG promoter, wherein the polynucleotide comprises a nucleotide sequence encoding a DAO1 enzyme described herein.

In some embodiments, the rAAV comprises an AAV9 capsid and an expression cassette comprising a polynucleotide operatively linked to a promoter (e.g., a CAG promoter or CMV promoter), wherein the polynucleotide comprises a nucleotide sequence encoding a DAO1 enzyme, wherein the DAO1 enzyme comprises an amino acid sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 1. In some embodiments, the DAO1 enzyme comprises the amino acid sequence set forth in SEQ ID NO: 1. In some embodiments, the rAAV comprises an AAV9 capsid and an expression cassette comprising a polynucleotide operatively linked to a promoter (e.g., a CAG promoter or CMV promoter), wherein the polynucleotide comprises a nucleotide encoding a DAO1 enzyme, wherein the nucleotide sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 30. In some embodiments, the nucleotide sequence comprises SEQ ID NO: 30.

In some embodiments, the rAAV comprises an AAV9 capsid and an expression cassette comprising a polynucleotide operatively linked to a promoter (e.g., a CAG promoter or CMV promoter), wherein the polynucleotide comprises a nucleotide sequence encoding a DAO1 enzyme, wherein the DAO1 enzyme comprises or consists of an amino acid sequence that shares at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 2. In some embodiments, the DAO1 enzyme comprises the amino acid sequence set forth in SEQ ID NO: 2. In some embodiments, the rAAV comprises an AAV9 capsid and an expression cassette comprising a polynucleotide operatively linked to a promoter (e.g., a CAG promoter or CMV promoter), wherein the polynucleotide comprises a nucleotide encoding a DAO1 enzyme, wherein the nucleotide sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 31. In some embodiments, the nucleotide sequence comprises SEQ ID NO: 31. In some embodiments, the rAAV comprises an AAV9 capsid and an expression cassette comprising a polynucleotide operatively linked to a promoter (e.g., a CAG promoter or CMV promoter), wherein the polynucleotide comprises a nucleotide encoding a DAO1 enzyme, wherein the nucleotide sequence comprises a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 29. In some embodiments, the nucleotide sequence comprises SEQ ID NO: 29.

In some embodiments, the rAAV comprises an AAV9 capsid and an expression cassette comprising or consisting of a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO: 23. In some embodiments, the rAAV comprises an AAV9 capsid and an expression cassette comprising or consisting of SEQ ID NO: 23.

### Methods of Use

Methods and compositions described herein can be used to treat ocular conditions and reduce the associated symptoms of the ocular conditions. The terms “treatment”, “treating” and the like are used herein to generally mean obtaining a desired pharmacologic and/or physiologic effect. The effect may be prophylactic in terms of completely or partially preventing a disease or symptom thereof, e.g., reducing the likelihood that the disease or symptom thereof occurs in the subject, and/or may be therapeutic in terms of a partial or complete cure for a disease and/or adverse effect attributable to the disease. “Treatment” as used herein covers any treatment of a disease in a mammal, and includes, without limitation: (a) inhibiting progress of the disease; (b) alleviating, reducing, or reducing an increase in one or more symptoms of the disease; (c) alleviating, reducing, or reducing an increase in one or more signs of the disease; (d) causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

As used herein, “administer,” “administering,” “administration” and the like refers to providing a substance (e.g., an rAAV vector, or a polypeptide comprising DAO1, or a fragment thereof) to a subject in a manner that is pharmacologically useful (e.g., to treat a disease, disorder, or condition in the subject).

In some embodiments, the ocular condition treated in accordance with the methods described herein associated with increased histamine production and/or increased histamine signaling. In some embodiments, the ocular condition is an inflammatory condition. In some embodiments, the ocular condition is an autoimmune reaction. In some embodiments, the ocular condition is an autoimmune condition. In some embodiments, the ocular condition is an allergic reaction. In some embodiments, the ocular condition is an allergic reaction to a therapeutic agent. In some embodiments, the ocular condition is an allergic condition. In some embodiments, the ocular condition is vernal keratoconjunctivitis. In some embodiments, the ocular condition is atopic keratoconjunctivitis. In some embodiments, the ocular condition is seasonal or perennial allergic conjunctivitis.

In some embodiments, the disclosure provides a method of treating a subject with an ocular disease or disorder, comprising administering to the subject an rAAV vector comprising a nucleotide encoding human DAO1 described herein, wherein expression of the human DAO1 is increased in the subject compared to expression of the human DAO1 in an untreated subject, or in the contralateral eye of a treated subject. Similarly, methods of treatment comprising the administration of a polypeptide comprising DAO1 or a fragment thereof, as well as the administration of alternative vectors (e.g., plasmids) configured to express DAO1 or a fragment thereof, are also described herein in various embodiments.

In some embodiments, the disclosure provides rAAV vectors for use in a method of treating an ocular condition in a subject in need thereof, the method comprising administering a recombinant adeno-associated virus (rAAV) vector, the rAAV vector comprising an AAV capsid and an expression cassette comprising a polynucleotide encoding human DAO1 operatively linked to a promoter, to at least one eye of the subject or to at least one lacrimal gland of the eye of the subject. In some embodiments, the rAAV vector for use is administered to the lacrimal gland of a subject. In some embodiments, the rAAV vector for use comprises an expression cassette encoding human DAO1.

In some embodiments, the disclosure provides rAAV vector for use, or adaptable for use, to treat a subject with an ocular disease, disorder, or condition. In some embodiments, the rAAV vector for use or adaptable for use comprise an AAV capsid and an expression cassette comprising a polynucleotide encoding human DAO1 (or a fragment thereof) operatively linked to a promoter.

In some embodiments, the disclosure provides methods of treating an ocular condition in a subject in need thereof, comprising administering a recombinant adeno-associated virus (rAAV) vector described herein, a plasmid configured to express DAO1 or a fragment thereof, or a polypeptide comprising DAO1 or a fragment thereof, to at least one eye of the subject or to at least one lacrimal gland of the eye of the subject. In some embodiments, the rAAV (or any other vector) being administered, may comprise a nucleotide encoding a DAO1 that shares at least 90% (e.g., 91, 92, 93, 94, 95, 96, 97, 98, 99, or 100%) sequence identity with SEQ ID NO: 1 or 2. Similarly, in embodiments where the DAO1 is administered directly, the polypeptide comprising the DAO1 may share at least 90% (e.g., 91, 92, 93, 94, 95, 96, 97, 98, 99, or 100%) sequence identity with SEQ ID NO: 1 or 2.

In some embodiments, the disclosure provides rAAV vectors for use in a method of treating an ocular condition in a subject in need thereof, the method comprising administering a recombinant adeno-associated virus (rAAV) vector comprising an expression cassette comprising the nucleic acid sequence of SEQ ID NO: 23 to at least one eye of the subject or to at least one lacrimal gland of the eye of the subject.

In some embodiments, expression of the human DAO1 is increased 5%, 10%, 15%, 20%, 25%, 30%, 35%, 40%, 45%, 50%, 55%, 60%, 65%, 70%, 75%, 80%, 85%, 90%, 95%, or 99% compared to expression of human DAO1 in an untreated subject, or in the contralateral eye of a treated subject. In some embodiments, expression of human DAO1 is increased by at least 1.5-fold, 2-fold, 3-fold, 4-fold, or 5-fold, 6-fold, 7-fold, 8-fold, or 9-fold compared to expression of human DAO1 in an untreated subject, or in the contralateral eye of a treated subject.

In some embodiments, expression of the human DAO1 is increased for about 1 day, 2 days, 3 days, 4 days, 5 days, 6 days, 7 days, 1 week, 2 weeks, 3 weeks, 4 weeks, 1 month, 2 months, 3 months, 4 months, 5 months, 6 months, 7 months, 8 months, 9 months, 10 months, 11 months, or 1 year compared to expression of the human DAO1 in an untreated subject, or in the contralateral eye of a treated subject.

As used herein, “approximately” or “about,” as applied to one or more values of interest, refers to a value that is similar to a stated reference value. In certain embodiments, “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1% or less in either direction (greater than or less than) of the stated reference value unless otherwise stated or otherwise evident from the context (except where such number would exceed 100% of a possible value).

In some embodiments, a subject is administered an rAAV or other vector comprising a nucleotide encoding human DAO1 enzyme described herein, wherein expression of the human DAO1 enzyme is increased in the subject compared to expression of human DAO1 in an untreated subject, or in the contralateral eye of a treated subject.

In some embodiments, expression of the human DAO1 enzyme is increased 5%, 10%, 15%, 20%, 25%, 30%, 35%, 40%, 45%, 50%, 55%, 60%, 65%, 70%, 75%, 80%, 85%, 90%, 95%, or 99% compared to expression of the human DAO1 enzyme in an untreated subject, or in the contralateral eye of a treated subject. In some embodiments, expression of the human DAO1 enzyme may be increased at least 1.5-fold, 2-fold, 3-fold, 4-fold, or 5-fold, 6-fold, 7-fold, 8-fold, or 9-fold compared to expression of the human DAO1 enzyme in an untreated subject, or in the contralateral eye of a treated subject.

In some embodiments, expression of the human DAO1 enzyme is increased for about 1 day, 2 days, 3 days, 4 days, 5 days, 6 days, 7 days, 1 week, 2 weeks, 3 weeks, 4 weeks, 1 month, 2 months, 3 months, 4 months, 5 months, 6 months, 7 months, 8 months, 9 months, 10 months, 11 months, or 1 year compared to expression of the human DAO1 enzyme in an untreated subject, or in the contralateral eye of a treated subject.

In some embodiments, the disclosure provides a method of treating a subject with an ocular disease or disorder, comprising administering to the subject DAO1, or an rAAV (or other vector) comprising a nucleotide encoding a human DAO1 enzyme, wherein expression of human DAO1 enzyme is increased in the subject compared to expression of human DAO1 enzyme in an untreated subject, or in the contralateral eye of a treated subject.

In some embodiments, the disclosure provides a method for treating a condition associated with histamine intolerance in a subject in need thereof, the method comprising administering to the subject an effective amount of DAO1, or an effective amount of an rAAV (or other vector) described herein. In some embodiments, the condition is associated with an overproduction of histamine in one or more tissues of the subject. In some embodiments, the condition is associated with an overproduction of histamine in one or both eyes of the subject. In some embodiments, the condition is associated with an accumulation of histamine in one or more tissues of the subject. In some embodiments, the condition is associated with an accumulation of histamine in one or both eyes of the subject.

In some embodiments, the histamine intolerance results from an allergic condition. In some embodiments, the allergic condition is a seasonal or perennial allergic condition. In some embodiments, the histamine intolerance results from an autoimmune condition.

In some embodiments, the condition is characterized by one or more allergy symptoms (e.g., sneezing, rhinitis, conjunctivitis, shortness of breath, cough, chest tightness, hives, skin discoloration, stomach cramps, throat constriction, pain, swelling, vomiting, diarrhea, bloating), wherein the administering ameliorates or prevents the one or more symptoms. In some embodiments, the condition is characterized by one or more symptoms of the eye (e.g., itching, swelling, tearing, and redness), wherein the administering ameliorates or prevents the one or more symptoms. In some embodiments, the severity of the one or more symptoms are evaluated based upon any method known in the art for characterizing allergy-related symptoms. In some embodiments, the severity of the one or more symptoms are evaluated based upon any method known in the art for characterizing a pathology associated with histamine intolerance. In some embodiments the one or more symptoms are evaluated using methods described herein (e.g., conjunctival allergen challenge, corneal staining test, eye dryness score test, Schirmer score test, ocular surface disease index test).

In some embodiments, the method comprises administering a dose of DAO1, or an rAAV (or another vector configured to express DAO1) to a subject having a condition associated with histamine intolerance (e.g., an allergic condition), wherein the condition is characterized by one or more symptoms described herein (e.g., one or more symptoms of an allergy). In some embodiments, the dose is administered prior to the onset of the one or more symptoms. In some embodiments, the administering prevents the onset of the one or more symptoms. In some embodiments, the administering reduces the severity of the one or more symptoms. For example, in some embodiments, the method comprises administering a dose of the rAAV to a subject having a seasonal or perennial allergic condition characterized by one or more symptoms (e.g., rhinitis, keratoconjunctivitis, headache, fatigue, cough, sneezing), wherein the dose of the rAAV is administered prior to the season during which the allergic condition occurs, wherein the administering prevents the onset of the one or more symptoms and/or reduces the severity of the one or more symptoms. In some embodiments, the dose is administered following the onset of the one or more symptoms, wherein the administering reduces the severity of the one or more symptoms.

In some embodiments, the method comprises administering a dosing regimen of the rAAV to a subject having a condition associated with a histamine intolerance (e.g., allergic condition), wherein the condition is characterized by one or more symptoms described herein (e.g., one or more symptoms of an allergy), wherein the dosing regimen comprises a first dose of the rAAV and at least one additional dose, wherein the first dose of the rAAV is administered to the subject prior to or subsequent to the onset of the one or more symptoms, and wherein the at least one additional dose is administered to the subject following the first dose, thereby preventing or reducing the severity of the one or more symptoms. In some embodiments, the dosing regimen comprises a frequency of dosing and/or dose amount that is selected based upon the pharmacokinetic parameters of the rAAV. In some embodiments, a clinician will administer the rAAV at a frequency and/or dose that achieves or maintains one or more desired effects. In some embodiments, the one or more desired effects is preventing one or more symptoms associated with the subject's condition. In some embodiments, the one or more desired effects is reducing the severity of one or more symptoms associated with the subject's condition. In some embodiments, the severity of the one or more symptoms is measured using a method described herein or known in the art for evaluating a pathology associated with histamine intolerance. In some embodiments, the one or more desired effects is achieved immediately following administering of the first dose of the rAAV. In some embodiments, the one or more desired effects occurs at any time point following administering of the first dose of the rAAV. In some embodiments, the one or more desired effects is achieved following administration of the at least one additional dose of the rAAV. In some embodiments, the one or more desired effects is achieved at any point during the dosing regimen. In some embodiments, the one or more desired effects is achieved following administration of the first dose of the rAAV and the subject is administered at least one additional dose of the rAAV to prevent reversal of the one or more desired effects. In some embodiments, the method comprises administering a first dose of the rAAV to the subject prior to, immediately following, or during the onset of one or more symptoms and an additional dose following a duration of about 1 week, about 2 weeks, about 3 weeks, about 1 month, about 2 months, about 3 months, about 4 months, about 5 months, about 6 months, about 7 months, about 8 months, about 9 months, about 10 months, about 11 months, about 1 year, about 1.5 years, or about 2 years after the first dose. The protocol and parameters described above may be used to design regimens for treatments wherein DAO1 is administered directly or via an alternative vector system (e.g., plasmid-based expression).

In some embodiments, the disclosure provides a method for treating an inflammatory condition of the eye in a subject in need thereof, the method comprising administering to the subject one or more therapeutically effective doses of DAO1, or an rAAV or other vector described herein.

In some embodiments, the disclosure provides a method for treating an autoimmune condition of the eye in a subject in need thereof, the method comprising administering to the subject one or more therapeutically effective doses of DAO1, or an rAAV or other vector described herein.

In some embodiments, the disclosure provides a method for treating an allergic condition of the eye in a subject in need thereof, the method comprising administering to the subject one or more therapeutically effective doses of DAO1, or an rAAV or other vector described herein.

In some embodiments, the disclosure provides a method for treating vernal keratoconjunctivitis in a subject in need thereof, the method comprising administering to the subject one or more therapeutically effective doses of DAO1, or an rAAV or other vector described herein.

In some embodiments, the disclosure provides a method for treating atopic keratoconjunctivitis in a subject in need thereof, the method comprising administering to the subject one or more therapeutically effective doses of DAO1, or an rAAV or other vector described herein.

In some embodiments, the disclosure provides a method for treating seasonal or perennial allergic conjunctivitis in a subject in need thereof, the method comprising administering to the subject one or more therapeutically effective doses of DAO1, or an rAAV or other vector described herein.

In some embodiments, the disclosure provides an rAAV described herein for use in a method of treating a condition associated with histamine intolerance in a subject in need thereof, the method comprising administering to the subject an effective amount of an rAAV described herein.

In some embodiments, the disclosure provides an rAAV described herein for use in the manufacture of a medicament for treating a condition associated with histamine intolerance in a subject in need thereof.

In some embodiments, the disclosure provides an rAAV described herein for use in a method of treating an autoimmune condition in a subject in need thereof, the method comprising administering to the subject an effective amount of an rAAV described herein.

In some embodiments, the disclosure provides an rAAV described herein for use in the manufacture of a medicament for treating an autoimmune condition in a subject in need thereof.

In some embodiments, the disclosure provides an rAAV described herein for use in a method of treating an allergy condition in a subject in need thereof, the method comprising administering to the subject an effective amount of an rAAV described herein.

In some embodiments, the disclosure provides an rAAV described herein for use in the manufacture of a medicament for treating an allergy condition in a subject in need thereof.

In some embodiments, the disclosure provides an rAAV described herein for use in a method of treating a vernal keratoconjunctivitis in a subject in need thereof, the method comprising administering to the subject an effective amount of an rAAV described herein.

In some embodiments, the disclosure provides an rAAV described herein for use in the manufacture of a medicament for treating a vernal keratoconjunctivitisin a subject in need thereof.

In some embodiments, the disclosure provides an rAAV described herein for use in a method of treating an atopic keratoconjunctivitis in a subject in need thereof, the method comprising administering to the subject an effective amount of an rAAV described herein.

In some embodiments, the disclosure provides an rAAV described herein for use in the manufacture of a medicament for treating an atopic keratoconjunctivitis in a subject in need thereof.

In some embodiments, the disclosure provides an rAAV described herein for use in a method of treating a seasonal or perennial allergic conjunctivitis in a subject in need thereof, the method comprising administering to the subject an effective amount of an rAAV described herein.

In some embodiments, the disclosure provides an rAAV described herein for use in the manufacture of a medicament for treating a seasonal or perennial allergic conjunctivitis in a subject in need thereof.

### Combination Therapy

In some embodiments, a method of treatment described herein comprises further administering to the subject one or more additional therapeutic agents. In some embodiments, the additional therapeutic agent is an agent that increases tear production. In some embodiments, the additional therapeutic agent is another AAV-based gene therapy construct. Suitable therapeutic agents for use as a combination therapy in the present disclosure are further described in PCT/US2022/027653, herein incorporated by reference.

In some embodiments, the disclosure provides a method for treating a condition associated with histamine intolerance in a subject in need thereof, the method comprising administering to one or both eyes of a subject an effective amount of DAO1, or an effective amount of an rAAV or other vector described herein, wherein the patient is receiving, has received, or will subsequently receive a treatment that increases tear production and/or re-establishes tear film homeostasis. In some embodiments, the subject has decreased tear production and/or impaired tear film homeostasis. In some embodiments, the DAO1, or the vector (e.g., the rAAV) is administered via intralacrimal injection to one or both lacrimal glands. In some embodiments, the DAO1, or the vector (e.g., the rAAV) is administered by topical application to the eye. In some embodiments, the treatment is administered following administration of the DAO1, or the vector (e.g., the rAAV) to the subject. In some embodiments, the treatment results in an increased amount of the DAO1, or the vector (e.g., the rAAV) delivered to the tear film of the eye of the subject in a predetermined time compared to the amount of DAO1 or the vector (e.g., the rAAV) delivered to the tear film in the absence of the subject receiving the treatment. In some embodiments, the predetermined period is about 5 minutes to about 60 minutes.

In some embodiments, the treatment that increases tear production comprises administering an effective amount of a nicotinic acetylcholine receptor (nAChR) agonist, or a pharmaceutically acceptable salt thereof. In some embodiments, the nAChR agonist is one described herein.

nAChRs are a class of pentameric ligand-gated ion channels that have high affinity and selectivity for both nicotine and acetylcholine (which resembles nicotine in its protonated form) and comprise combinations of alpha and beta subunits. Examples of nAChR subtypes include, but are not limited to, alpha3beta4, alpha4beta2, alpha3alpha5beta4, and alpha4alpha6beta2. An important nAChR receptor subtype involved in instigating the nasolacrimal reflex, for example, is the alpha4beta2 subtype located on the trigeminal nerve endings in the nasal mucosa.

Administration of a nAChR agonist may desensitize the receptor. Receptor desensitization results in reduced response to an agonist even at higher agonist concentrations, which further results in diminished efficacy of the treatment. For instance, short term desensitization of the nAChR receptor to an agonist may occur over a 24-hour period after administration of the agonist. The potential for receptor desensitization may potentially limit the dosing frequency over a period of time in order to preserve an effective response to the agonist.

A nAChR agonist may be characterized as a full or partial agonist as determined by its ability to activate a given receptor to produce a response as compared to the response at that receptor for acetylcholine (ACh). In general, a nAChR agonist is a full agonist if it evokes a response upon binding to a given receptor that is equal or greater to that of ACh. A nAChR agonist is a partial agonist if it evokes a lower response upon binding to the receptor as compared to the response generated from ACh.

nAChR agonist response, from which receptor activation can be determined can, for example, be generated using an appropriate cell-based assay. Cells designed to express a particular nAChR receptor subtype and generate an electrical current response when bound to and activated by a nAChR agonist can be used to characterize the agonist profile of a compound and the amount of receptor activation thus determined. An example of a generic protocol is described below.

Cells that express a particular human nAChR subtype are first exposed to ACh. ACh binds and activates the receptor, thereby evoking a current. The concentration of the ACh is chosen to elicit the maximum response of the receptor (e.g., 1280 micromolar ACh). This current is recorded as the ACh response and serves as the 100% nAChR agonist response and to which responses to other nAChR agonists are compared. After washing, the cells are exposed to a nAChR agonist at various concentrations (e.g., 0.1, 0.3, 1, 3, 10, 30, 100, and 300 micromolar).

The current evoked by exposure to the nAChR agonist is measured and recorded for each nAChR concentration. This nAChR agonist response data is then normalized to unity versus the maximal ACh evoked current and plotted as a function of the logarithm of the nAChR agonist concentration. The nAChR agonist response is then calculated as a percentage of the ACh response.

In some embodiments, the method to determine the relative agonist activity of nAChR agonist comprises conditions wherein the ACh response is evoked from a 1 or more millimolar ACh solution.

A nAChR agonist evoking a response equal to or greater than the maximum ACh response determined at the same receptor type is a full agonist. In some embodiments, a nAChR agonist evoking a response of less than 100% of the ACh response may still be characterized a full agonist, taking into account experimental variability. For example, variability between tests or measurement methods, and statistical error, may account for differences in the response results. In some embodiments, a nAChR agonist evoking 80% to 120% of the ACh response is considered a full agonist. In some embodiments, a nAChR agonist evoking 99% of the ACh response or greater is considered a full agonist. In some embodiments, a nAChR agonist evoking 95% of the ACh response or greater is considered a full agonist. In some embodiments, a nAChR agonist evoking 90% of the ACh response or greater is considered a full agonist. In some embodiments, a nAChR agonist evoking 85% of the ACh response or greater is considered a full agonist. In some embodiments, a nAChR agonist evoking 80% of the ACh response or greater is considered a full agonist.

Taking into account experimental variability, if the nAChR agonist evokes less than 100% of the ACh response, then generally the agonist is considered a partial agonist. In some embodiments, a nAChR agonist evoking less than 95% of the ACh response is considered a partial agonist. In some embodiments, a nAChR agonist evoking less than 90% of the ACh response is considered a partial agonist. In some embodiments, a nAChR agonist evoking less than 85% of the ACh response is considered a partial agonist. In some embodiments, a nAChR agonist evoking less than 80% of the ACh response is considered a partial agonist.

In some embodiments, a nAChR agonist evoking 5% to 95% of the ACh response is considered a partial agonist. In some embodiments, a nAChR agonist evoking 5% to 90% of the ACh response is considered a partial agonist. In some embodiments, a nAChR agonist evoking 5% to 85% of the ACh response is considered a partial agonist. In some embodiments, a nAChR agonist evoking 5% to 80% of the ACh response is considered a partial agonist.

In some embodiments, a nAChR agonist evoking 10% to 95% of the ACh response is considered a partial agonist. In some embodiments, a nAChR agonist evoking 10% to 90% of the ACh response is considered a partial agonist. In some embodiments, a nAChR agonist evoking 10% to 85% of the ACh response is considered a partial agonist. In some embodiments, a nAChR agonist evoking 10% to 80% of the ACh response is considered a partial agonist.

nAChR agonists that generate a low level of electrical activity at relatively high concentrations of agonist may be described as a weak partial agonist. In some embodiments, a nAChR agonist evoking 30% or less of the ACh response is considered a weak partial agonist. In some embodiments, a nAChR agonist evoking 25% or less of the ACh response is considered a weak partial agonist. In some embodiments, a nAChR agonist evoking 20% or less of the ACh response is considered a weak partial agonist. In some embodiments, the relatively high concentration of nAChR agonist is at least 100 micromolar. In some embodiments, the relatively high concentration of nAChR agonist is at least 200 micromolar. In some embodiments, the relatively high concentration of nAChR agonist is at least 300 micromolar or greater. For instance, a 300 micromolar concentration of nAChR agonist that evokes 25% of the maximal Ach-evoked current is considered a weak partial agonist.

In some embodiments, the nAChR agonist is a full agonist. In some embodiments, the nAChR agonist is a partial agonist. In some embodiments, the nAChR agonist is a weak partial agonist.

In some embodiments, the nAChR agonist, or a pharmaceutically acceptable salt thereof, is an agonist of at least one of the nAChR subtypes selected from alpha3beta4, alpha3alpha5beta4, alpha4beta2, and alpha4alpha6beta2. In some embodiments, the nAChR agonist, or a pharmaceutically acceptable salt thereof, is an agonist of at least two of the nAChR subtypes selected from alpha3beta4, alpha3alpha5beta4, alpha4beta2, and alpha4alpha6beta2. In some embodiments, the nAChR agonist, or a pharmaceutically acceptable salt thereof, is an agonist of at least three of the nAChR subtypes selected from alpha3beta4, alpha3alpha5beta4, alpha4beta2, and alpha4alpha6beta2. In some embodiments, the nAChR agonist, or a pharmaceutically acceptable salt thereof, is an agonist of nAChR subtype alpha3beta4. In some embodiments, the nAChR agonist, or a pharmaceutically acceptable salt thereof, is an agonist of nAChR subtype alpha3alpha5beta4. In some embodiments, the nAChR agonist, or a pharmaceutically acceptable salt thereof, is an agonist of nAChR subtype alpha4beta2. In some embodiments, the nAChR agonist, or a pharmaceutically acceptable salt thereof, is an agonist of nAChR subtype alpha4alpha6beta2. In some embodiments, the nAChR agonist, or a pharmaceutically acceptable salt thereof, is an agonist of nAChR subtype alpha7. In some embodiments, the nAChR agonist, or a pharmaceutically acceptable salt thereof, is not an agonist of nAChR subtype alpha7. In some embodiments, the nAChR agonist, or a pharmaceutically acceptable salt thereof, is not a full agonist of nAChR subtype alpha7. In some embodiments, the nAChR agonist is a full agonist of the aforementioned subtypes. In some embodiments, the nAChR agonist is a partial agonist of the aforementioned subtypes. In some embodiments, the nAChR agonist is a weak partial agonist of the aforementioned subtypes.

The terms “alpha7” or “07” nAChR refer to the homomeric alpha7 subtype, wherein the pentameric subunits of nAChR are composed entirely of alpha7 subunits. Thus, a nAChR agonist that binds and activates nAChR alpha7 is an agonist that binds and activates nAChR homomeric alpha7 receptor. In some embodiments described herein, the nAChR agonist is not an alpha7 receptor agonist.

In some embodiments, the nAChR agonist, or a pharmaceutically acceptable salt thereof, selectively binds to at least one of the nAChR subtypes selected from alpha3beta4, alpha3alpha5beta4, alpha4beta2, and alpha4alpha6beta2. As used herein, “selectively binds” or “is selective for” means that a compound has a higher affinity for the nAChR subtype and/or a lower half-maximal effective concentration (“EC50”) for that nAChr subtype for at least one reference nAChR subtype. Selectivity may be associated with at least a 5-fold affinity difference in EC50 value, at least a 10-fold affinity difference in EC50 value, at least a 20-fold affinity difference in EC50 value, or at least a 50-fold affinity difference in EC50 value. In some embodiments, the nAChR agonist, or a pharmaceutically acceptable salt thereof, selectively binds to nAChR subtype alpha3beta4. In some embodiments, the nAChR agonist, or a pharmaceutically acceptable salt thereof, selectively binds to nAChR subtype alpha3alpha5beta4. In some embodiments, the nAChR agonist, or a pharmaceutically acceptable salt thereof, selectively binds to nAChR subtype alpha4beta2. In some embodiments, the nAChR agonist, or a pharmaceutically acceptable salt thereof, selectively binds to nAChR subtype alpha4alpha6beta2. In some embodiments, the nAChR agonist, or a pharmaceutically acceptable salt thereof, selectively binds to nAChR subtype alpha7. In some embodiments, the nAChR agonist, or a pharmaceutically acceptable salt thereof, does not selectively bind to nAChR subtype alpha7.

The nAChR agonists contemplated in this disclosure include varenicline, a pharmaceutically acceptable salt thereof, and compound 1, or a pharmaceutically acceptable salt thereof. In some embodiments the nAChR agonist is not varenicline.

Varenicline is characterized as a full agonist of the nAChR subtype alpha7 and a partial agonist of subtypes alpha3beta4, alpha4beta2, alpha6beta2, alpha3alpha5beta4, and alpha4alpha6beta2. In some embodiments, the nAChR agonist is varenicline, or a pharmaceutically acceptable salt thereof. Pharmaceutically acceptable salts of varenicline include varenicline tartrate. Additional related information for varenicline may be found in, for example, U.S. Pat. Nos. 9,504,644, 9,504,645, 9,532,944, 9,597,284, and 10,456,386.

Compound 1, as recited herein, refers to the structure:

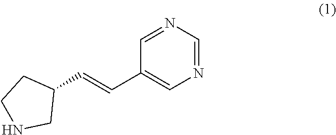 [SMILES: C(=C\[C@H]1CCNC1)/c1cncnc1]

An alternative structural representation of compound 1 is shown here:

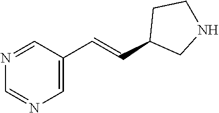 [SMILES: C(=C\[C@H]1CCNC1)/c1cncnc1]

Compound 1 may be also referred to by its chemical name. For instance, compound 1 is also referred to as (R)-5-((E)-2-pyrrolidin-3-ylvinyl)pyrimidine, or variations thereof including simpanicline, 5-{(E)-2-[(3R)-pyrrolidin-3-yl]vinyl}pyrimidine and (R,E)-5-((2-pyrrolidine-3-yl)vinyl)pyrimidine.

Compound 1 is a full agonist of nAChR subtypes alpha4beta2, alpha3beta4, alpha3alpha5beta4, and alpha4alpha6beta2. Compound 1 is a full agonist of nAChR subtypes alpha4beta2, and alpha3beta4.

Compound 1 is a partial agonist of subtype alpha3beta2.

Compound 1 is a weak partial agonist of subtype alpha7. In one example, a 300 micromolar concentration of compound 1 citrate evoked only 25% of the maximal ACh-evoked current.

In some embodiments, the nAChR agonist may be compound 1, or a pharmaceutically acceptable salt thereof. Pharmaceutically acceptable salts of compound 1 include galactarate (e.g., hemi-galactarate dihydrate) and citrate (e.g., mono-citrate). Patent related information for compound 1 may be found in U.S. Pat. Nos. 7,098,331, 7,714,001, 8,063,068, 8,067,443, 8,604,191, 9,145,396, 9,981,949, 8,633,222, and PCT publication WO 2017/177024.

In some embodiments, the nAChR agonist is (R)-5-((E)-2-pyrrolidin-3-ylvinyl)pyrimidine, or a pharmaceutically acceptable salt thereof. In some embodiments, the nAChR agonist is (R)-5-((E)-2-pyrrolidin-3-ylvinyl)pyrimidine hemigalactarate dihydrate. In some embodiments, the nAChR agonist is (R)-5-((E)-2-pyrrolidin-3-ylvinyl)pyrimidine mono-citrate.

### Modes of Administration

In some embodiments, the disclosure provides methods comprising administering an rAAV vector, the rAAV vector comprising an AAV capsid and an expression cassette comprising a polynucleotide encoding DAO1 operatively linked to a promoter, to the eye of the subject or to a lacrimal gland of the eye of the subject. In other aspects, methods according to the disclosure may comprise administration of a polypeptide comprising DAO1 or a fragment thereof, or an alternative vector configured to express DAO1 or a fragment thereof (e.g., plasmid-based expression systems) to the eye of the subject or to a lacrimal gland of the eye of the subject.

As noted above, the lacrimal functional unit is composed of main and accessory lacrimal glands, the ocular surface, and the interconnecting innervation. For each eye, a main lacrimal gland is situated superotemporally in the orbital within the lacrimal fossa of the frontal bone. The accessory glands, known as the Wolfring's glands and Krause's glands are located in the eyelid. In the upper eyelid there are about 2 to 5 Wolfring's glands and about forty Krause's glands. In the lower eyelid there are about 6 to 8 Krause's glands. Specific location and anatomy of the lacrimal functional unit is well-known (Conrady et al. J Ophthalmol.; 2016: 7542929 (2016)). Together, the lacrimal glands secrete tear film onto the ocular surface through lacrimal ducts. Lacrimal glands also express and secrete proteins and products necessary for corneal regeneration and promoting transparency into the tear film, such as transforming growth factor-O and retinol (Conrady et al. J Ophthalmol.; 2016: 7542929 (2016); Pan et al. Optom Vis Sci.; 95:27-31 (2018)). In addition to secreting tears into the eye, the lacrimal duct drains the lacrimal fluid into the nasal cavity.

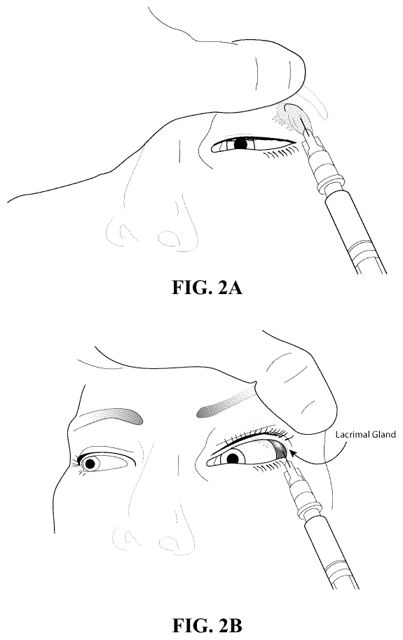

Administration of DAO1, or of a vector (e.g., an rAAV) to the lacrimal gland may be accomplished by topical administration to the ocular surface, direct injection into the lacrimal gland, and/or topical administration to the lacrimal gland. In some embodiments, the DAO1 or the vector (e.g., the rAAV) is administered to the lacrimal gland by topical administration. In some embodiments, the DAO1 or the vector (e.g., the rAAV) is administered to the lacrimal gland by direct injection. The lacrimal gland may be accessed surgically or by manipulation of the eyelid. Manipulation of the eyelid provides access to the tissue for administration topically (e.g., by lavage of the tissue with a pharmaceutical composition comprising the viral vector). Direct injection into the lacrimal gland may be done by penetrating the skin over the lacrimal gland (FIG. 2A) or by manipulating the eyelid to access the lacrimal gland (FIG. 2B). Administration of a viral vector to the lacrimal gland may be accomplished by transconjunctival injection. In some embodiments, the rAAV is administered to the lacrimal gland by direct injection as depicted in FIG. 2A. In some embodiments, the rAAV is administered to the lacrimal gland by manipulating the eyelid as depicted in FIG. 2B.

In some embodiments, cells within the eye, the lacrimal gland, and/or the nasolacrimal duct are transduced by the rAAV vector. Cells within the eye, the lacrimal gland and/or the nasolacrimal duct include, without limitation, acinar cells, ductal cells, and/or myoepithelial cells. In some embodiments, the transduced cells within the eye, the lacrimal gland, and/or the nasolacrimal duct express a therapeutically effective amount of DAO1 enzyme into the tear film and optionally onto the ocular surface of the subject. In some embodiments, a therapeutically effective amount of DAO1 enzyme is secreted into the nasal cavity of the subject. In some embodiments, a therapeutically effective amount of DAO1 enzyme is secreted onto the ocular surface of the subject.

Delivery of rAAV vectors to the eye and/or lacrimal gland to express a transgene into tear film has been demonstrated in vivo. In one example, the main lacrimal glands of mice were directly injected with rAAV vectors encoding a luciferase transgene with serotypes AAV2, AAV4, AAV5, AAV 5w8, AAV x5, AAV 9, AAV12, and bovine AAV (BAAV). AAV9, AAV 5w8, AAV5, and AAV2 each are able to transduce the lacrimal ductal and acinar cells of the lacrimal gland (Rocha et al., supra).

In some embodiments, the rAAV vector is administered to a lacrimal gland of the subject. In some embodiments, the lacrimal gland is the main lacrimal gland. In some embodiments, the lacrimal gland is any one of the Wolfring's glands or the Krause's glands of the subject.

Compositions and rAAV vectors of the disclosure may be administered to the lacrimal gland of the subject by any suitable method. For example, the subject composition may be administered by direct injection to the main or accessory lacrimal glands.

Access to the lacrimal gland in human subjects can be achieved, for example, by manually elevating the upper eyelid to expose the palpebral lobe of the lacrimal gland and delivering the therapeutic agent using a syringe, e.g., with a 30G needle.

The viral vectors of the disclosure are generally delivered to the subject as a pharmaceutical composition. Pharmaceutical compositions comprise a pharmaceutically acceptable solvent (e.g., water, etc.) and one or more excipients. In some embodiments, the pharmaceutical compositions comprise a buffer at about neutral pH (pH 5, 6, 7, 8, or 9). In some embodiments, the pharmaceutical composition comprises phosphate buffered saline (e.g., PBS at pH of about 7). The pharmaceutical compositions may comprise a pharmaceutically acceptable salt. The concentration of the salt may be selected to ensure that the pharmaceutical composition is isotonic to, or nearly isotonic to, the target tissue.

In some embodiments, the pharmaceutical compositions of the disclosure comprise about 1×10⁸ genome copies per milliliter (GC/mL), about 5×10⁸ GC/mL, about 1×10⁹ GC/mL, about 5×10⁹ GC/mL, about 1×10¹⁰ GC/mL, about 5×10¹¹ GC/mL, about 1×10¹¹ GC/mL, about 5×10¹¹ GC/mL, about 1×10¹² GC/mL, about 5×10¹² GC/mL, about 5×10³ GC/mL, or about 1×10¹⁴ GC/mL of the viral vector (e.g., rAAV vector). In some embodiments, the pharmaceutical compositions of the disclosure comprise about 1×10⁸ genome copies per milliliter (GC/mL), about 5×10⁸ GC/mL to about 1×10⁹ GC/mL, about 1×10⁹ GC/mL to about 5×10⁹ GC/mL, about 5×10⁹ GC/mL to about 1×10¹⁰ GC/mL, about 1×10¹⁰ GC/mL to about 5×10¹⁰ GC/mL, about 5×10¹⁰ GC/mL to about 1×10¹¹ GC/mL, about 1×10¹¹ GC/mL to about 5×10¹¹ GC/mL, about 5×10¹¹ GC/mL to about 1×10¹² GC/mL, about 1×10¹² GC/mL to about 5×10¹² GC/mL, about 5×10¹² GC/mL to about 5×10¹³ GC/mL, or about 5×10¹³ GC/mL to about 1×10¹⁴ GC/mL of the viral vector (e.g., rAAV vector). In some embodiments, the pharmaceutical compositions of the disclosure comprise about 5×10⁸ GC/mL to about 5×10⁹ GC/mL, about 5×10⁹ GC/mL to about 5×10¹⁰ GC/mL, about 5×10¹⁰ GC/mL to about 5×10¹¹ GC/mL, about 5×10¹¹ GC/mL to about 5×10¹² GC/mL, or about 5×10¹² GC/mL to about 1×10¹⁴ GC/mL of the viral vector (e.g., rAAV vector). In yet further embodiments, the pharmaceutical compositions of the disclosure comprise about 5×10⁸ GC/mL to about 5×10¹⁰ GC/mL, about 5×10¹⁰ GC/mL to about 5×10¹² GC/mL, or about 5×10¹² GC/mL to about 1×10¹⁴ GC/mL of the viral vector (e.g., rAAV vector).

In some embodiments, the pharmaceutical compositions of the disclosure comprise about 1×10¹² GC/mL to about 6.2×10¹² GC/mL of the viral vector (e.g., rAAV vector). In some embodiments, the pharmaceutical compositions of the disclosure comprise about 1×10¹² GC/mL or about 6.2×10¹² GC/mL of the viral vector (e.g., rAAV vector).

In some embodiments, the pharmaceutical compositions of the disclosure are administered in a total volume of about 10 μL, about 20 μL, about 30 μL, about 40 μL, about 50 μL, about 60 μL, about 70 μL, about 80 μL, about 90 μL, about 100 μL, 110 μL, about 120 μL, about 130 μL, about 140 μL, about 150 μL, about 160 μL, about 170 μL, about 180 μL, about 190 μL, or about 200 μL. In some embodiments, the pharmaceutical compositions of the disclosure are administered in a total volume of about 10 μL to about 20 μL, about 20 μL to about 30 μL, about 30 μL to about 40 μL, about 40 μL to about 50 μL, about 50 μL to about 60 μL, about 60 μL to about 70 μL, about 70 μL to about 80 μL, about 80 μL to about 90 μL, about 90 μL to about 100 μL, about 100 μL to 110 μL, 110 μL to about 120 μL, about 120 μL to about 130 μL, about 130 μL to about 140 μL, about 140 μL to about 150 μL, about 150 μL to about 160 μL, about 160 μL to about 170 μL, about 170 μL to about 180 μL, about 180 μL to about 190 μL, or about 190 μL to about 200 μL.

Genome copies per milliliter can be determined by quantitative polymerase change reaction (qPCR) using a standard curve generated with a reference sample having a known concentration of the polynucleotide genome of the virus. For AAV, the reference sample used is often the transfer plasmid used in generation of the rAAV vector but other reference samples may be used.

Alternatively, or in addition, the concentration of a viral vector can be determined by measuring the titer of the vector on a cell line. Viral titer is typically expressed as viral particles (vp) per unit volume (e.g., vp/mL). In some embodiments, the pharmaceutical compositions of the disclosure comprise about 1×10⁸ viral particles per milliliter (vp/mL), about 5×10⁸ vp/mL, about 1×10⁹ vp/mL, about 5×10⁹ vp/mL, about 1×10¹⁰ vp/mL, about 5×10¹⁰ vp/mL, about 1×10¹¹ vp/mL, about 5×10¹¹ vp/mL, about 1×10¹² vp/mL, about 5×10¹² vp/mL, about 5×10¹³ vp/mL, or about 1×10¹⁴ vp/mL of the viral vector (e.g., rAAV vector). In some embodiments, the pharmaceutical compositions of the disclosure comprise about 1×10⁸ viral particles per milliliter (vp/mL) to about 5×10⁸ vp/mL, about 5×10⁸ vp/mL to about 1×10⁹ vp/mL, about 1×10⁹ vp/mL to about 5×10⁹ vp/mL, about 5×10⁹ vp/mL to about 1×10¹⁰ vp/mL, about 1×10¹⁰ vp/mL to about 5×10¹⁰ vp/mL, about 5×10¹⁰ vp/mL to about 1×10¹¹ vp/mL, about 1×10¹¹ vp/mL to about 5×10¹¹ vp/mL, about 5×10¹¹ vp/mL to about 1×10¹² vp/mL, about 1×10¹² vp/mL to about 5×10¹² vp/mL, about 5×10¹² vp/mL to about 5×10¹³ vp/mL, or about 5×10¹³ vp/mL to about 1×10¹⁴ vp/mL of the viral vector (e.g., rAAV vector).

A variety of tests are available to evaluate ocular conditions in a subject before, during, and after treatment with any of the methods or compositions disclosed herein. In some of the embodiments disclosed herein, the effective treatment of the subject is indicated by one or more of the tests that can be, for example, a) Eye Dryness score test on a visual analog scale, b) Schirmer's test, c) Corneal Fluorescein Staining test, and d) Ocular Surface Disease Index test. Tests to evaluate the signs and symptoms of an ocular condition may be administered under standardized or reproducible conditions in order to obtain a subject's test score. Conditions include exposing the subject to an environment artificially created to adversely challenge the subject or where the environment (temperature, humidity, air flow) is monitored and carefully controlled.

### Assessment of Efficacy

The efficacy of a method described herein can be assessed using any suitable method known in the art.

In some embodiments, the methods described herein result in one or more symptoms of the ocular condition being reduced compared to the symptoms of the ocular condition before administration of the rAAV vector. As used herein, “symptoms” include any of the diagnostic criteria or symptoms associated with a given ocular condition, including those described herein. Non-limiting examples of symptoms that may be alleviated by treatment in accordance with the methods described herein include, for example, itching, swelling, tearing, and redness.

In some embodiments, symptoms (e.g., itching, swelling, tearing, and redness) can be reduced following administration of the compositions and rAAVs of the disclosure. In some embodiments, one or more symptoms of the ocular condition are reduced compared to the symptoms of the ocular condition in an untreated control subject. In some embodiments, one or more symptoms of the ocular condition are reduced compared to the symptoms of the ocular condition in a contralateral eye. By “contralateral eye” it is meant the eye of the subject that is opposite from the eye that has been treated with a composition according to the present disclosure. The contralateral eye can be used as a control for treatment, so long as the subject suffers from bilateral disease, or in the case of a model animal, has been subjected to the experimental protocol leading up to treatment in both eyes.

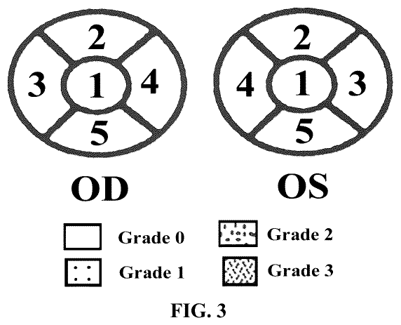

The efficacy of a method of treatment described herein may be evaluated by assessing the symptoms of an ocular condition treated in accordance with the methods described herein on a clinical scale. Examples of the clinical scales that may be used to evaluate the efficacy of a method of treatment described herein include, without limitation, the Conjunctival Itching Grading Scale (Table 4), the Conjunctival Redness Assessment Grading Scale (Table 5), and the Corneal Staining Grading Scale (FIG. 3).

As used herein, the term “statistically significant” refers to a method of analysis selected by a person of ordinary skill in the art based upon study design and the data type generated to assess the difference observed between two or more groups, wherein the analysis determines if the difference is not random or due strictly to chance.

### Conjunctival Allergen Challenge (CAC)

A conjunctival allergen challenge consists of an initial visit—the allergen titration visit—increasing allergen concentrations of the specific allergen that elicited a positive skin test reaction are instilled bilaterally at 10-minute intervals until a positive reaction is induced. Following challenge, ocular/conjunctival itching is evaluated by the subject, and ocular/conjunctival redness is evaluated by trained clinicians; both evaluations use standardized rating scales that range from 0 (none) to 4 (severe). A positive CAC reaction is defined as moderate to severe redness in two of the three vessel beds (conjunctival, ciliary, episcleral) of each eye and moderate to severe itching in both eyes within 10 minutes of allergen instillation. Subjects who fail to test positively are excluded from the study. During the following visit, the allergen confirmation, patients are challenged with the final titer of allergen used in the first visit. If a moderate to severe ocular allergic response is reproduced at two out of the three post-CAC time points, the diagnosis is confirmed, and the subject is enrolled.

### Conjunctival Itching Grading Scale

In some embodiments, treatment of an ocular condition in accordance with the methods described herein results in an improvement of 0.5 points, 1 point, 1.5 points, 2 points, 2.5 points, 3 point, 3.5 points, or 4 points on the Conjunctival Itching Grading Scale shown in Table 4.

TABLE 4
Conjunctival Itching Grading Scale.

| Score | Ocular Itching Descriptor |
|---|---|
| 0 | None |
| 0.5 | An intermittent tickle sensation possible |
| | localized in the corner of the eye |
| 1.0 | An intermittent tickle sensation involving |
| | more than just the corner of the eye |
| 1.5 | Intermittent all-over tickling sensation |
| 2.0 | A mild continuous itch (can be localized) without desire to rub |
| 2.5 | Moderate, diffuse continuous itch with desire to rub |
| 3.0 | A severe itch with desire to rub |
| 3.5 | Severe itch improved with minimal rubbing |
| 4.0 | Incapacitating itch with an irresistible urge to rub |

In some of the embodiments disclosed herein, effective treatment is indicated by a statistically significant decrease in the subject's score on the Conjunctival Itching Grading Scale, and wherein the statistically significant decrease in the subject's score on the Conjunctival Itching Grading Scale is determined after administration to the subject of the first dose, or the optionally one or more subsequent doses, of the rAAV vector described herein, wherein the subject's score on the Conjunctival Itching Grading Scale is compared to a) a score on the Conjunctival Itching Grading Scale of the subject prior to administration of the first dose of the rAAV vector; b) a score on the Conjunctival Itching Grading Scale of an subject administered a control; c) a score on the Conjunctival Itching Grading Scale of an subject administered a comparator compound; or d) a contralateral eye.

In some of the embodiments disclosed herein, the subject's score on the Conjunctival Itching Grading Scale is compared to a corneal score of the subject prior to administration of the rAAV vector according to the methods described herein. In some of the embodiments disclosed herein, the subject's score on the Conjunctival Itching Grading Scale is compared to a score on the Conjunctival Itching Grading Scale of a subject administered a control. In some of the embodiments disclosed herein, the subject's score on the Conjunctival Itching Grading Scale is compared to a score on the Conjunctival Itching Grading Scale of a subject administered a comparator compound. In some of the embodiments disclosed herein, the subject's score on the Conjunctival Itching Grading Scale is compared to a contralateral eye.

In some embodiments, a statistically significant decrease in a subject's ocular/conjunctival itching score is observed in a subject administered an rAAV vector described herein compared to a control. In some of the embodiments described herein, the statistically significant decrease in the subject's ocular/conjunctival itching score is at least 5%, 10%, 15%, 20%, 25%, 50%, 75%, 80%, 90%, 95%, 100%, 125%, 150%, 200%, 250%, 300%, or 350%. In some embodiments, a statistically significant decrease in a subject's ocular/conjunctival itching score is observed in a subject administered an rAAV vector described herein compared to a control, wherein the statistically significant decrease in the subject's ocular/conjunctival itching score is at least 5%, 10%, 15%, 20%, 25%, 50%, 75%, 80%, 90%, 95%, 100%, 125%, 150%, 200%, 250%, 300%, or 35%.

In some of the embodiments described herein, the statistically significant decrease in the subject's ocular/conjunctival itching score is characterized by a p value of 0.05 or less, 0.01 or less, 0.005 or less, or 0.001 or less. In some of the embodiments described herein, the statistically significant decrease in the subject's ocular/conjunctival itching score is characterized by a p value of 0.05 or less. In some of the embodiments described herein, the statistically significant decrease in the subject's ocular/conjunctival itching score is characterized by a p value of 0.01 or less.

In some of the embodiments described herein, the statistically significant decrease in the subject's ocular/conjunctival itching score is within 1 day, within 2 days, within 3 days, within 4 days, within 1 week, within 2 weeks, within 3 weeks, within 4 weeks, within 1 month, within 2 months, within 3 months, within 4 months, within 5 months or within 6 months after treatment with any of the methods or compositions disclosed.

In some embodiments, the decrease in a subject's ocular/conjunctival itching score persists for about 1 day, 2 days, 3 days, 4 days, 5 days, 6 days, 7 days, 1 week, 2 weeks, 3 weeks, 4 weeks, 1 month, 2 months, 3 months, 4 months, 5 months, 6 months, 9 months, or 1 year after treatment with any of the methods or compositions disclosed herein.

In some of the embodiments described herein, wherein the statistically significant decrease in the subject's ocular/conjunctival itching score is based on the subject's ocular/conjunctival itching score determined after administering the first dose of the rAAV vector.

In some of the embodiments described herein, the statistically significant decrease is based on the subject's ocular/conjunctival itching score determined after treatment with any of the methods or compositions disclosed herein.

### Conjunctival Redness Assessment Grading Scale

In some embodiments, treatment of an ocular condition in accordance with the methods described herein results in an improvement of 1 point, 2 points, 3 point, or 4 points on the Conjunctival Redness Assessment Grading Scale shown in Table 5.

TABLE 5
Conjunctival Redness Assessment Grading Scale

| Score | Ocular Redness Descriptor |
|---|---|
| 0 | None |
| 1 | Mild: slightly dilated blood vessels; color of |
| | vessels is typically pink; can be quadrantal |
| 2 | Moderate: more apparent dilation of blood vessels; |
| | vessel color is more intense (redder); |
| | involves the majority of the vessel bed |
| 3 | Severe: numerous and obvious dilated blood vessels; |
| | in the absence of chemosis the color is deep red, |
| | may be less red or pink in presence of chemosis, |
| | is not quadrantic |
| 4 | Extremely severe: large, numerous, dilated blood |
| | vessels characterized by unusually severe deep red |
| | color, regardless of grade of chemosis, which |
| | involves the entire vessel bed |

In some of the embodiments disclosed herein, effective treatment is indicated by a statistically significant decrease in the subject's score on the Conjunctival Redness Assessment Grading Scale, and wherein the statistically significant decrease in the subject's score on the Conjunctival Redness Assessment Grading Scale is determined after administration to the subject of the first dose, or the optionally one or more subsequent doses, of the rAAV vector described herein, wherein the subject's score on the Conjunctival Redness Assessment Grading Scale is compared to a) a score on the Conjunctival Redness Assessment Grading Scale of the subject prior to administration of the first dose of the rAAV vector; b) a score on the Conjunctival Redness Assessment Grading Scale of an subject administered a control; c) a score on the Conjunctival Redness Assessment Grading Scale of an subject administered a comparator compound; or d) a contralateral eye.

In some of the embodiments disclosed herein, the subject's ocular/conjunctival redness score is compared to an ocular/conjunctival redness score of the subject prior to administration of the first dose of the rAAV vector, and the first dose of the treatment that increases tear production.

In some of the embodiments disclosed herein, the subject's ocular/conjunctival itching score is compared to an ocular/conjunctival redness score of a subject administered a control.

In some of the embodiments disclosed herein, the subject's ocular/conjunctival itching score is compared to an ocular/conjunctival redness score of a subject administered a comparator compound.

In some of the embodiments disclosed herein, the subject's ocular/conjunctival redness score is compared to an ocular/conjunctival itching score in a contralateral eye.

In some embodiments, a statistically significant decrease in a subject's ocular/conjunctival redness score is observed in a subject administered an rAAV vector described herein compared to a control. In some of the embodiments described herein, the statistically significant decrease in the subject's ocular/conjunctival redness score is at least 5%, 10%, 15%, 20%, 25%, 50%, 75%, 80%, 90%, 95%, 100%, 125%, 150%, 200%, 250%, 300%, or 350%. In some embodiments, a statistically significant decrease in a subject's ocular/conjunctival redness score is observed in a subject administered an rAAV vector described herein compared to a control, wherein the statistically significant decrease in the subject's ocular/conjunctival redness score is at least 5%, 10%, 15%, 20%, 25%, 50%, 75%, 80%, 90%, 95%, 100%, 125%, 150%, 200%, 250%, 300%, or 350%.

In some of the embodiments described herein, the statistically significant decrease in the subject's ocular/conjunctival redness score is characterized by a p value of 0.05 or less, 0.01 or less, 0.005 or less, or 0.001 or less. In some of the embodiments described herein, the statistically significant decrease in the subject's ocular/conjunctival redness score is characterized by a p-value of 0.05 or less. In some of the embodiments described herein, the statistically significant decrease in the subject's ocular/conjunctival redness score is characterized by a p-value of 0.01 or less.

In some of the embodiments described herein, the statistically significant decrease in the subject's ocular/conjunctival redness score is within 1 day, within 2 days, within 3 days, within 4 days, within 1 week, within 2 weeks, within 3 weeks, within 4 weeks, within 1 month, within 2 months, within 3 months, within 4 months, within 5 months or within 6 months after treatment with any of the methods or compositions disclosed.

In some embodiments, the decrease in a subject's ocular/conjunctival redness score persists for about 1 day, 2 days, 3 days, 4 days, 5 days, 6 days, 7 days, 1 week, 2 weeks, 3 weeks, 4 weeks, 1 month, 2 months, 3 months, 4 months, 5 months, 6 months, 9 months, or 1 year after treatment with any of the methods or compositions disclosed herein.

In some of the embodiments described herein, wherein the statistically significant decrease in the subject's ocular/conjunctival redness score is based on the subject's ocular/conjunctival redness score determined after administering the first dose of the rAAV vector.

In some of the embodiments described herein, the statistically significant decrease is based on the subject's ocular/conjunctival redness score determined after treatment with any of the methods or compositions disclosed herein.

In some of the embodiments disclosed herein, the subject's score on the Conjunctival Redness Assessment Grading Scale is compared to a corneal score of the subject prior to administration of the rAAV vector according to the methods described herein. In some of the embodiments disclosed herein, the subject's score on the Conjunctival Redness Assessment Grading Scale is compared to a score on the Conjunctival Redness Assessment Grading Scale of a subject administered a control. In some of the embodiments disclosed herein, the subject's score on the Conjunctival Redness Assessment Grading Scale is compared to a score on the Conjunctival Redness Assessment Grading Scale of a subject administered a comparator compound. In some of the embodiments disclosed herein, the subject's score on the Conjunctival Redness Assessment Grading Scale is compared to a contralateral eye.

In some embodiments, treatment of an ocular condition in accordance with the methods described herein results in an improvement of about 10% to about 20%, about 20% to about 30%, about 30% to about 40%, about 40% to about 50%, about 50% to about 60%, about 60% to about 70%, about 70% to about 80%, about 80% to about 90%, or more than about 95% in at least one area on the Corneal Staining Grading Scale.

Corneal surface changes are associated with insufficient tear flow and excessive dryness, as well as the ocular conditions described herein and ocular discomfort. Corneal surface changes may include disruption of the mucin coating protecting the surface epithelial cells, and/or damage to the epithelial cell walls.

### Corneal Staining Test

Corneal surface changes are associated with insufficient tear flow and excessive dryness, as well as the ocular conditions described herein and ocular discomfort. Corneal surface changes may include disruption of the mucin coating protecting the surface epithelial cells, and/or damage to the epithelial cell walls.

Corneal Staining, including fluorescein staining, lissamine green staining, and rose bengal staining, tests are diagnostic tests for determining corneal surface health and can indicate areas of damage on the corneal surface. Any suitable method of corneal staining known in the art may be used to evaluate the efficacy of a method of treatment described herein, for example those described in Begley et al., The Ocular Surface 17 (2019) 208-220. The normal corneal surface does not take up water-soluble dyes instilled into the tear film. However, damaged epithelial cells allow water-soluble dyes to diffuse into the surface cells. The dyes, which stain damaged epithelial cells, may be visualized on the corneal surface indicating damage on the corneal surface.

To administer a Corneal Staining test, a staining dye is applied to one or both eyes. The dye is allowed to penetrate and stain the area between surface cells. Devitalized cells and strands of devitalized surface tissue (filaments) can be visualized with this test. A test administrator then uses a corneal surface scoring system developed to rate the severity of damage observed. This scoring system is useful for treatment of an ocular condition over time. FIG. 3 depicts the NEI/Industry Workshop Scale used in the scoring system. Other equivalent standardized scoring systems may be used. A test administrator grades the areas of the corneal surface that are damaged and calculates the corneal score reflecting the severity of damage to the corneal surface.

A test administrator may use the corneal score to evaluate the effectiveness of a particular treatment in a subject. The test may be administered multiple times to monitor any change in the severity of the subject's ocular surface over a period of time. In general, higher numbers indicate more damage to the corneal surface compared to lower numbers, which indicate lower levels of damage to the corneal surface. Reduction in corneal scores over time is evidence of a reduction in the damage to corneal surface. The decrease in corneal scores generally indicates and improvement in the subject's condition. The decrease in corneal scores over time is also evidence that the treatment is effective in treating dry eye disease, increasing tear production, or improving ocular discomfort.

In some of the embodiments disclosed herein, effective treatment is indicated by a statistically significant decrease in the subject's corneal score, and wherein the statistically significant decrease in the subject's corneal score is determined after administration to the subject of the first dose, or the optionally one or more subsequent doses, of the compositions described herein, wherein the subject's corneal score is compared to a) a corneal score of the subject prior to administration of the first dose of the rAAV vector, and the first dose of the treatment that increases tear production; b) a corneal score of an subject administered a control; c) a corneal score of an subject administered a comparator compound; or d) a contralateral eye.

In some of the embodiments disclosed herein, the subject's corneal score is compared to a corneal score of the subject prior to administration of the pharmaceutical composition according to the methods described herein. In some of the embodiments disclosed herein, the subject's corneal score is compared to a corneal score of a subject administered a control. In some of the embodiments disclosed herein, the subject's corneal score is compared to a corneal score of a subject administered a comparator compound. In some of the embodiments disclosed herein, the subject's corneal score is compared to a contralateral eye.

In some of the embodiments disclosed herein, the subject's corneal score may be used to measure a definition of corneal healing defined as less than 0.5-mm fluorescein staining or no (zero) fluorescein staining (Bonini et al., Ophthalmology. 125:1332-1343 (2018)).

In some embodiments, a statistically significant decrease in a subject's corneal healing score is observed in a subject administered an rAAV vector described herein compared to a control.

In some of the embodiments described herein, the statistically significant decrease in the subject's corneal healing score is at least 5%, 10%, 15%, 20%, 25%, 50%, 75%, 80%, 90%, 95%, 100%, 125%, 150%, 200%, 250%, 300%, or 350%. In some embodiments, a statistically significant decrease in a subject's OSDI score is observed in a subject administered an rAAV vector described herein compared to a control, wherein the statistically significant decrease in the subject's OSDI Score is at least 5%, 10%, 15%, 20%, 25%, 50%, 75%, 80%, 90%, 95%, 100%, 125%, 150%, 200%, 250%, 300%, or 350%.

In some of the embodiments described herein, the statistically significant decrease in the subject's corneal staining score is at least 5%, 10%, 15%, 20%, 25%, 50%, 75%, 80%, 90%, 95%, 100%, 125%, 150%, 200%, 250%, 300%, or 350%.

In some of the embodiments described herein, the statistically significant decrease in the subject's corneal score is characterized by a p value of 0.05 or less, 0.01 or less, 0.005 or less, or 0.001 or less.

In some of the embodiments described herein, the statistically significant decrease in the subject's corneal score is within 1 day, within 2 days, within 3 days, within 4 days, within 1 week, within 2 weeks, within 3 weeks, within 4 weeks, within 1 month, within 2 months, within 3 months, within 4 months, within 5 months or within 6 months after treatment according to the methods or compositions disclosed herein.

In some embodiments, the decrease in a subject's corneal score persists for at least 1 day, 2 days, 3 days, 4 days, 5 days, 6 days, 7 days, 1 week, 2 weeks, 3 weeks, 4 weeks, 1 month, 2 months, 3 months, 4 months, 5 months, 6 months, 9 months, or 1 year after treatment with any of the methods or compositions disclosed herein.

In some of the embodiments described herein, the statistically significant decrease is based on the subject's corneal score determined after administering one or more subsequent doses of the rAAV vector, and optionally the first or the one or more subsequent doses of the treatment that increases tear production.

### Eye Dryness Score Test

An Eye Dryness Score test on a visual analog scale may be used to evaluate ocular conditions, tearing levels, or ocular discomfort in a subject. The test involves the use of a visual analog scale and comprises a 100 mm horizontal line where one endpoint at 0 is labeled “no discomfort” and the other endpoint at 100 is labeled “maximal discomfort.” FIG. 3 shows an example of the visual analog scale (not shown at actual scale). The subject is asked to rate their ocular symptoms due to eye dryness by placing a vertical mark on the horizontal line to indicate their level of discomfort. The Eye Dryness score is then obtained by identifying where the subject's response is on the 100 mm scale.

The Eye Dryness score, measured in mm, can be used to evaluate the severity of the ocular symptom, and the effectiveness of a particular treatment of a subject. This test has the advantage of being administered to the subject as frequently as every 5 minutes, which allows the test administrator to closely monitor the changes in the subject's symptoms over time. Higher numbers indicate more discomfort for an ocular symptom compared to lower numbers, which indicate relatively lower levels of discomfort. A decrease in the Eye Dryness score is evidence that the treatment is effective in treating an ocular condition, increasing tear production, or improving ocular discomfort. Reduction in Eye Dryness scores over time is evidence of a reduction in or alleviation of the ocular symptom and generally indicates an improvement in the subject's condition.

In some of the embodiments disclosed herein, effective treatment is indicated by a statistically significant decrease in the subject's Eye Dryness score, and wherein the statistically significant decrease in the subject's Eye Dryness score is determined after administration to the subject of the first dose, or the optionally one or more subsequent doses, of the rAAV vector, and the first dose, or the optionally one or more subsequent doses, of the treatment that increases tear production, wherein the subject's Eye Dryness score is compared to a) an Eye Dryness score of the subject prior to administration of the first dose of the rAAV vector, and the first dose of the treatment that increases tear production; b) an Eye Dryness score of a subject administered a control; c) an Eye Dryness score of a subject administered a comparator compound; or d) an Eye Dryness score in a contralateral eye.

In some of the embodiments disclosed herein, the subject's Eye Dryness score is compared to an Eye Dryness score of the subject prior to administration of the first dose of the rAAV vector, and the first dose of the treatment that increases tear production.

In some of the embodiments disclosed herein, the subject's Eye Dryness score is compared to an Eye Dryness score of a subject administered a control.

In some of the embodiments disclosed herein, the subject's Eye Dryness score is compared to an Eye Dryness score of a subject administered a comparator compound.

In some of the embodiments disclosed herein, the subject's Eye Dryness score is compared to an Eye Dryness score in a contralateral eye.

In some embodiments, a statistically significant decrease in a subject's Eye Dryness score is observed in a subject administered an rAAV vector described herein compared to a control. In some of the embodiments described herein, the statistically significant decrease in the subject's Eye Dryness Score is at least 5%, 10%, 15%, 20%, 25%, 50%, 75%, 80%, 90%, 95%, 100%, 125%, 150%, 200%, 250%, 300%, or 350%. In some embodiments, a statistically significant decrease in a subject's Eye Dryness score is observed in a subject administered an rAAV vector described herein compared to a control, wherein the statistically significant decrease in the subject's Eye Dryness Score is at least 5%, 10%, 15%, 20%, 25%, 50%, 75%, 80%, 90%, 95%, 100%, 125%, 150%, 200%, 250%, 300%, or 350%.

In some of the embodiments described herein, the statistically significant decrease in the subject's Eye Dryness score is at least 3 mm, at least 5 mm, at least 10 mm, at least 15 mm, at least 20 mm, at least 25 mm, at least 30 mm, at least 35 mm, at least 40 mm, at least 45 mm, or at least 50 mm. In some embodiments, a statistically significant decrease in a subject's Eye Dryness score is observed in a subject administered an rAAV vector described herein compared to a control, wherein the statistically significant decrease in the subject's Eye Dryness Score is at least 3 mm, at least 5 mm, at least 10 mm, at least 15 mm, at least 20 mm, at least 25 mm, at least 30 mm, at least 35 mm, at least 40 mm, at least 45 mm, or at least 50 mm.

In some of the embodiments described herein, the statistically significant decrease in the subject's Eye Dryness score is between 3 mm and 10 mm, between 3 mm and 20 mm, between 3 mm and 25 mm, between 3 mm and 30 mm, between 3 mm and 35 mm, between 3 mm and 40 mm, between 3 mm and 45 mm, between 3 mm and 50 mm, between 5 mm and 10 mm, between 5 mm and 20 mm, between 5 mm and 25 mm, between 5 mm and 30 mm, between 5 mm and 35 mm, between 5 mm and 40 mm, between 5 mm and 45 mm, between 5 mm and 50 mm, between 10 mm and 15 mm, between 10 mm and 20 mm, between 10 mm and 25 mm, between 10 mm and 30 mm, between 10 mm and 35 mm, between 10 mm and 40 mm, between 10 mm and 45 mm, between 10 mm and 50 mm, between 15 mm and 20 mm, between 20 mm and 30 mm, between 25 mm and 35 mm, between 30 mm and 40 mm, between 30 mm and 45 mm, or between 30 mm and 50 mm.

In some of the embodiments described herein, the statistically significant decrease in the subject's Eye Dryness Score is characterized by a p value of 0.05 or less, 0.01 or less, 0.005 or less, or 0.001 or less. In some of the embodiments described herein, the statistically significant decrease in the subject's Eye Dryness Score is characterized by a p value of 0.05 or less. In some of the embodiments described herein, the statistically significant decrease in the subject's Eye Dryness Score is characterized by a p value of 0.01 or less.

In some of the embodiments described herein, the statistically significant decrease in the subject's Eye Dryness Score is within 1 day, within 2 days, within 3 days, within 4 days, within 1 week, within 2 weeks, within 3 weeks, within 4 weeks, within 1 month, within 2 months, within 3 months, within 4 months, within 5 months or within 6 months after treatment with any of the methods or compositions disclosed.

In some embodiments, the decrease in a subject's Eye Dryness Score persists for about 1 day, 2 days, 3 days, 4 days, 5 days, 6 days, 7 days, 1 week, 2 weeks, 3 weeks, 4 weeks, 1 month, 2 months, 3 months, 4 months, 5 months, 6 months, 9 months, or 1 year after treatment with any of the methods or compositions disclosed herein.

In some of the embodiments described herein, wherein the statistically significant decrease in the subject's Eye Dryness score is based on the subject's Eye Dryness score determined after administering the first dose of the rAAV vector.

In some of the embodiments described herein, the statistically significant decrease is based on the subject's Eye Dryness score determined after treatment with any of the methods or compositions disclosed herein.

### Schirmer Score Test

Ocular conditions may affect tear volume and tear production. A Schirmer's test may be used to evaluate tear production and assess the severity of dry eye disease, insufficient tearing, or ocular discomfort in a subject. The test measures the amount of tears produced in each eye. The test typically involves first placing an anesthetic into one or both of the subject's eyes. These drops prevent the eyes from watering in reaction to the test strips. Then, the test administrator places a piece of filter paper inside one or both lower eyelids and the person closes their eyes. After 5 minutes, the test administrator removes the filter paper and assesses how far the tears have travelled on the paper. The Schirmer's test may be administered to one or both eyes.

In general, the smaller the amount of moisture on the paper, the fewer tears that person has produced. In healthy eyes, each strip of paper typically contains more than 10 millimeters of moisture. A Schirmer's score of less than 10 millimeters of moisture may indicate one or more of the following conditions including the ocular conditions described herein, abnormally low tearing, or ocular discomfort.

A Schirmer's test may be used to evaluate the effectiveness of a particular treatment in a subject and may be administered multiple times to monitor any change in the severity of the subject's symptoms over a period of time. An increase in Schirmer's scores over time in an subject being treated for ocular conditions described herein, insufficient tearing, or ocular discomfort is evidence for an increase in tear volume or tear production and generally indicates improvement in the subject's condition. An increase in the Schirmer's score over time is evidence that treatment is effective in treating ocular conditions described herein, increasing tear production, or improving ocular discomfort.

In some of the embodiments disclosed herein, effective treatment is indicated by a statistically significant increase in the subject's Schirmer's score, and wherein the statistically significant increase in the subject's Schirmer's score is determined after treatment with any of the methods or compositions disclosed herein, wherein the subject's Schirmer's score is compared to a) a Schirmer's score of the subject prior to administration of the first dose of the rAAV vector, and the first dose of the treatment that increases tear production; b) a Schirmer's score of a subject administered a control; c) a Schirmer's score of a subject administered a comparator compound; or d) a contralateral eye.

In some of the embodiments disclosed herein, the subject's Schirmer's score is compared to a Schirmer's score of the subject prior to administration of a pharmaceutical composition described herein. In some of the embodiments disclosed herein, the subject's Schirmer's score is compared to a Schirmer's score of a subject administered a control. In some of the embodiments disclosed herein, the subject's Schirmer's score is compared to a Schirmer's score of a subject administered a comparator compound. In some of the embodiments disclosed herein, the subject's Schirmer's score is compared to a contralateral eye.

In some embodiments, a statistically significant increase in a subject's Schirmer's score is observed in a subject administered an rAAV vector described herein compared to a control. In some of the embodiments described herein, the statistically significant increase in the subject's Schirmer's score is at least 5%, 10%, 15%, 20%, 25%, 50%, 75%, 80%, 90%, 95%, 100%, 125%, 150%, 200%, 250%, 300%, or 350%. In some of the embodiments described herein, the increase in the subject's Schirmer's score is at least 100%, 200%, or 300%. In some embodiments, a statistically significant increase in a subject's Schirmer's score is observed in a subject administered an rAAV vector described herein compared to a control, wherein the statistically significant increase in the subject's Schirmer's Score is at least 5%, 10%, 15%, 20%, 25%, 50%, 75%, 80%, 90%, 95%, 100%, 125%, 150%, 200%, 250%, 300%, or 350%.

In some of the embodiments described herein, the statistically significant increase in the subject's Schirmer's score is at least 3 mm, at least 5 mm, at least 10 mm, at least 15 mm, at least 20 mm, at least 25 mm, at least 30 mm, at least 35 mm, at least 40 mm, at least 45 mm, or at least 50 mm.

In some of the embodiments described herein, the statistically significant increase in the subject's Schirmer's score is between 3 mm and 5 mm, between 3 mm and 10 mm, between 3 mm and 15 mm, between 3 mm and 20 mm, between 3 mm and 25 mm, between 3 mm and 30 mm, between 5 mm and 10 mm, between 5 mm and 15 mm, between 5 mm and 20 mm, between 5 mm and 25 mm, between 5 mm and 30 mm, between 10 mm and 15 mm, between 10 mm and 20 mm, between 10 mm and 25 mm, between 10 mm and 30 mm, 15 mm and 20 mm, between 15 mm and 25 mm, between 15 mm and 30 mm, between 20 mm and 25 mm, or between 20 mm and 30 mm.

In some embodiments, a statistically significant increase in a subject's Schirmer score is observed in a subject administered an rAAV vector described herein compared to a control, wherein the statistically significant increase in the subject's Schirmer Score is at least 3 mm, at least 5 mm, at least 10 mm, at least 15 mm, at least 20 mm, at least 25 mm, at least 30 mm, at least 35 mm, at least 40 mm, at least 45 mm, or at least 50 mm.

In some of the embodiments described herein, the statistically significant decrease in the subject's Schirmer's score is characterized by a p value of 0.05 or less, 0.01 or less, 0.005 or less, or 0.001 or less.

In some of the embodiments described herein, the statistically significant decrease in the subject's Schirmer's score is within 1 day, within 2 days, within 3 days, within 4 days, within 1 week, within 2 weeks, within 3 weeks, within 4 weeks, within 1 month, within 2 months, within 3 months, within 4 months, within 5 months or within 6 months after treatment according to the methods or compositions disclosed herein.

In some of the embodiments described herein, wherein the statistically significant decrease in the subject's Schirmer's score is based on the subject's Schirmer's score determined after treatment according to the methods or compositions disclosed herein.

In some of the embodiments described herein, the statistically significant decrease is based on the subject's Schirmer's score determined after treatment according to any of the methods or compositions disclosed herein.

### Ocular Surface Disease Index Test

Subjects treated for ocular conditions and associated symptoms can provide important information used to diagnose their condition and determine the severity of symptoms through questionnaires. A well-designed questionnaire can be validated for reproducibility and should consist of relevant questions that elicit responsive answers. One example of a questionnaire is the Ocular Surface Disease Index (OSDI), a 12-question survey for subjects with an ocular condition that's been shown to be a reliable and valid instrument for directly assessing symptom frequency. The ocular symptoms that are evaluated include, but are not limited to, burning/stinging, itching, foreign body sensation, eye discomfort, eye dryness, photophobia, and pain. Most people are familiar with questionnaires and understand that they are an efficient way for a health provider to gather information. Questionnaires also reduce bias, as there are no verbal or visual cues to inadvertently influence the respondent. The test administrator collects the response from the subject and calculates an OSDI based on the subject's answers to the questions.

The OSDI score can be used to evaluate the severity of the ocular symptom, and the effectiveness of a particular treatment of a subject. Higher numbers indicate a higher severity of an ocular condition. Reduction in OSDI scores over time is evidence of a reduction in or alleviation of the ocular symptoms and generally indicates an improvement in the subject's condition. A decrease in the OSDI score is also evidence that the treatment is effective in treating an ocular conditions and reducing symptoms.

In some of the embodiments disclosed herein, effective treatment is indicated by a statistically significant decrease in the subject's OSDI score, and wherein the statistically significant decrease in the subject's OSDI score is determined after administration to the subject of the first dose, or the optionally one or more subsequent doses, of the rAAV vector, and the first dose, or the optionally one or more subsequent doses, of the treatment that increases tear production, wherein the subject's OSDI score is compared to a) an OSDI score of the subject prior to administration of the first dose of the rAAV vector, and the first dose of the treatment that increases tear production; b) an OSDI score of an subject administered a control; c) an OSDI score of a subject administered a comparator compound; or d) a contralateral eye.

In some of the embodiments disclosed herein, the subject's OSDI score is compared to an OSDI score of the subject prior to treatment according to the disclosed methods described herein. In some of the embodiments disclosed herein, the subject's OSDI score is compared to an OSDI score of a subject administered a control. In some of the embodiments disclosed herein, the subject's OSDI score is compared to an OSDI score of a subject administered a comparator compound. In some of the embodiments disclosed herein, the subject's OSDI score is compared to an OSDI score of a contralateral eye.

In some embodiments, a statistically significant decrease in a subject's OSDI score is observed in a subject administered an rAAV vector described herein compared to a control.

In some of the embodiments described herein, the statistically significant decrease in the subject's OSDI score is at least 5%, 10%, 15%, 20%, 25%, 50%, 75%, 80%, 90%, 95%, 100%, 125%, 150%, 200%, 250%, 300%, or 350%. In some embodiments, a statistically significant decrease in a subject's OSDI score is observed in a subject administered an rAAV vector described herein compared to a control, wherein the statistically significant decrease in the subject's OSDI Score is at least 5%, 10%, 15%, 20%, 25%, 50%, 75%, 80%, 90%, 95%, 100%, 125%, 150%, 200%, 250%, 300%, or 350%.

In some of the embodiments described herein, the statistically significant decrease in the subject's OSDI score is characterized by a p value of 0.05 or less, 0.01 or less, 0.005 or less, or 0.001 or less.

In some of the embodiments described herein, the statistically significant decrease in the subject's OSDI score is within 1 day, within 2 days, within 3 days, within 4 days, within 1 week, within 2 weeks, within 3 weeks, within 4 weeks, within 1 month, within 2 months, within 3 months, within 4 months, within 5 months or within 6 months after treatment according to the methods or compositions disclosed herein.

In some embodiments, the decrease in a subject's OSDI score persists for at least 1 day, 2 days, 3 days, 4 days, 5 days, 6 days, 7 days, 1 week, 2 weeks, 3 weeks, 4 weeks, 1 month, 2 months, 3 months, 4 months, 5 months, 6 months, 9 months, or 1 year after treatment with any of the methods or compositions disclosed herein.

In some of the embodiments described herein, wherein the statistically significant decrease in the subject's OSDI score is based on the subject's OSDI score determined after administering the first dose of the rAAV vector.

In some of the embodiments described herein, the statistically significant decrease is based on the subject's OSDI score determined after treatment according to the methods or compositions disclosed herein.

### Maintenance Of Effective Treatment Over Time

The efficacy of a method of treatment described herein may be evaluated at any suitable time point after administration, for example, the improvement on the Conjunctival Itching Grading Scale, the Conjunctival Redness Assessment Grading Scale, the Ocular Tearing Score Grading Scale, and/or the Corneal Staining Grading Scale may be measured at about 1 month, about 2 months, about 3 months, about 4 months, about 5 months, about 6 months, about 7 months, about 8 months, about 9 months, about 10 months, about 11 months, about 12 months, about 18 months, about 24 months, about 3 years, about 4 years or about 5 years after the administration of an rAAV vector described herein. In some embodiments, the improvement on the Conjunctival Itching Grading Scale, the Conjunctival Redness Assessment Grading Scale, the Ocular Tearing Score Grading Scale, and/or the Corneal Staining Grading Scale is measured at two or more time points after the administration of an rAAV vector described herein, for example, every 3 months, every 6 months or every 12 months after the administration of the rAAV vector.

The present disclosure may provide for effective treatment over a period of time where a statistically significant improvement in a subject's score is maintained. The term “maintained” as used in the present disclosure and as it relates to the maintenance of a statistically significant improvement in a subject's score (EDS, Schirmer, corneal staining, or OSDI) refers to the statistically significant improvement not diminishing below a certain threshold over time. A statistically significant improvement can be maintained even if, at a later point in time, the subject's score changes. An improvement after treatment according to the disclosed methods, can be maintained without additional dosing or after one or more subsequent doses.

For example, a corneal staining score of 0.5-mm or 0-mm could be defined as corneal healing and represent a significant improvement at 30 or 60 days after initiation of therapy (Bonini et al., Ophthalmology. 125:1332-1343 (2018)).

For example, an Eye Dryness score being “maintained within 10% means” that the decrease in subject's Eye Dryness score does not diminish by more than 10% during the specified time. A further improvement in the subject's Eye Dryness score would also be considered maintenance of the statistically significant improvement (e.g., if the Eye Dryness score further improved by 15% during the specified time, this would be considered “maintained within 10%”).

In another example, if there is a statistically significant decrease (improvement) in a subject's Eye Dryness score 30 days after treatment according to the methods described herein, and at a later point, the subject's score is the same or lesser (indicating a benefit to the subject), then the statistically significant improvement is said to be maintained. Alternatively, if at a later point, the subject's Eye Dryness score is greater than the Eye Dryness score 30 days after treatment according to the methods described herein, the subject may still be receiving a therapeutic benefit and the later determined score may still be a statistically significant improvement compared to prior administration of the first dose, or their pre-treatment baseline score.

In another example, if there is a statistically significant decrease (improvement) in a subject's Eye Dryness score 60 days after treatment according to the methods described herein, and at a later point, the subject's score is the same or lesser (indicating a benefit to the subject), then the statistically significant improvement is said to be maintained. Alternatively, if at a later point, the subject's Eye Dryness score is greater than the Eye Dryness score 60 days after treatment according to the methods described herein, the subject may still be receiving a therapeutic benefit and the later determined score may still be a statistically significant improvement compared to prior to treatment according to the methods described herein.

Note that what constitutes an improvement differs depending on the score being measured. For example, an improvement in an Eye Dryness score, the corneal score and the OSDI score is a decrease in the numerical value of the score. For the Schirmer's test, an improvement is typically an increase in the numerical value of the Schirmer's score.

In some of the embodiments described herein, the maintenance of the statistically significant improvement of the subject's score (e.g., EDS, Schirmer, corneal staining, or OSDI) means that the statistically significant improvement does not diminish by more than 10%, 20%, 30%, 40%, 50%, or 60%.

In some of the embodiments described herein, the statistically significant improvement in the subject's score (e.g., EDS, Schirmer, corneal, or OSDI) is maintained for at least 1 week, at least 1 month, at least 3 months, at least 6 months, at least 9 months, or at least 12 months. In some of the embodiments described herein, the statistically significant improvement in the subject's score is maintained for at least 30 days after treatment according to the methods described herein, wherein the statistically significant improvement does not diminish by more than 30%. In some embodiments, the statistically significant improvement in the subject's score is maintained for at least 1 month from administration of the first dose of the rAAV vector, and, optionally, the first dose of the treatment that increases tear production, wherein the statistically significant improvement does not diminish by more than 30%.

In some of the embodiments described herein, the statistically significant improvement in the subject's score (e.g., EDS, Schirmer, corneal, or OSDI) is maintained for at least 1 week, at least 1 month, at least 3 months, at least 6 months, at least 9 months, or at least 12 months after treatment according to the methods described herein, wherein the statistically significant improvement does not diminish by more than 10%, 20%, 30%, 40%, 50%, or 60% compared to the subject's score within 60 days after treatment according to the methods described herein.

In some of the embodiments described herein, the statistically significant improvement in the subject's score (e.g., EDS, Schirmer, corneal staining, or OSDI) is maintained for at least 1 week, at least 1 month, at least 3 months, at least 6 months, at least 9 months, or at least 12 months after after treatment according to the methods described herein, wherein the statistically significant improvement does not diminish by more than 20% compared to the subject's corresponding Eye Dryness Score, corneal score, or OSDI score within 60 days after treatment according to the methods described herein.

In some embodiments, the improvement measured on the Conjunctival Itching Grading Scale, the Conjunctival Redness Assessment Grading Scale, the Ocular Tearing Score Grading Scale, and the Corneal Staining Grading Scale persists for a prolonged time (e.g., for at least 3 months, at least 6 months, at least 9 months, at least 12 months, at least 15 months, at least 18 months, at least 24 months, at least 3 years, at least 4 years, or at least 5 years) after the administration of the rAAV vector provided herein.

### Dosing Timing And Method Of Administration

The schedule of doses administered to a subject depends on various considerations including the duration of effectiveness of each dose, the transduction efficiency of the rAAV vector, and the effect of the dose on the body. For example, wherein the patient's condition does not improve, upon the health provider's discretion, the method of treating an ocular condition as described herein, may be adjusted in dose or administered repeatedly in order to ameliorate or otherwise control or limit the symptoms of the subject's ocular condition. For instance, the period of time between administrations of one or more doses is extended, or the period of time between days the subject is administered one or more doses is extended. As a non-limiting example, administration of one or more doses is modified to administration of one or more doses after measuring symptoms of the ocular condition.

The term “dose”, as used herein, may refer to a dose of a pharmaceutical composition of the disclosure, or a dose of the treatment that reduces symptoms in an ocular condition.

In some of the embodiments described herein, a dose of the rAAV vector is a dose of a rAAV vector carrying an expression cassette. In such cases, delivery of an appropriate dose (e.g., effective amount) of the gene product is achieved by administering an appropriate amount/titer of the viral vector to the target site which allows expression of an effective amount of the gene product over a period of time. An “effective amount,” as used herein, refers to an amount or dose of an rAAV, treatment, or composition described herein that is sufficient to reduce the symptoms and or signs of an ocular condition described herein. The term “amount” as used herein refers to an absolute amount (e.g., an absolute amount of protein or rAAV particles) or concentration (e.g., a concentration of protein in a solution), whether the amount referred to in a given instance refers to an absolute amount, concentration, or both, will be clear to the skilled artisan based on the context provided herein.

In some embodiments, the viral vector is an rAAV vector. In some embodiments, the viral vector is administered to the lacrimal gland. In some embodiments, the rAAV is administered to the lacrimal gland by topical administration. In some embodiments, the rAAV is administered to the lacrimal gland by direct injection. In some embodiments, a dose of the rAAV results in the stable production of the gene product for a period of time (e.g., about 1 day, about 2 days, about 4 days, about 1 week, about 2 weeks, about 3 weeks, about 4 weeks, about 1 month, about 2 months, about 3 months, about 4 months, about 5 months, about 6 months, about 9 months, about 12 months, or longer). In some embodiments, a dose of the rAAV results in the stable production of the gene product for about 1 week. In some embodiments, a dose of the rAAV results in the stable production of the gene product for about 2 weeks. In some embodiments, a dose of the rAAV results in the stable production of the gene product for about 3 weeks. In some embodiments, a dose of the rAAV results in the stable production of the gene product for about 4 weeks. In some embodiments, a dose of the rAAV results in the stable production of the gene product for about 1 month. In some embodiments, a dose of the rAAV results in the stable production of the gene product for about 2 months. In some embodiments, a dose of the rAAV results in the stable production of the gene product for about 3 months. In some embodiments, a dose of the rAAV results in the stable production of the gene product for about 4 months. In some embodiments, a dose of the rAAV results in the stable production of the gene product for about 5 months. In some embodiments, a dose of the rAAV results in the stable production of the gene product for about 6 months. In some embodiments, a dose of the rAAV results in the stable production of the gene product for about 9 months. In some embodiments, a dose of the rAAV results in the stable production of the gene product for about 12 months.

In some embodiments, a method described herein comprises administering an effective amount of an rAAV described herein to a subject, wherein the rAAV comprises a polynucleotide encoding a gene product (e.g., DAO1). In some embodiments, the method comprises delivering a first dose and one or more subsequent doses of the rAAV. The one or more subsequent doses are administered after a period of time after the first dose. In some embodiments, this period of time between the first dose and the next subsequent dose is at least 1 day, at least 3 days, at least 1 week, at least 2 weeks, at least 3 weeks, at least 4 weeks, at least 1 month, at least 2 months, at least 4 months, at least 6 months, at least 9 months, at least 12 months, or longer. In some embodiments, this period of time between the first dose and the next subsequent dose is between 1-7 days, between 1-4 weeks, between 2-6 weeks, between 4-8 weeks, between 1-3 months, between 2-4 months, between 3-6 months, between 4-12 months, between 6-24 months. In some embodiments, the period of time between the one or more subsequent doses is at least 1 day, at least 3 days, at least 1 week, at least 2 weeks, at least 3 weeks, at least 4 weeks, at least 1 month, at least 2 months, at least 4 months, at least 6 months, at least 9 months, at least 12 months, or longer. In some embodiments, the period of time between the one or more subsequent doses is between 1-7 days, between 1-4 weeks, between 2-6 weeks, between 4-8 weeks, between 1-3 months, between 2-4 months, between 3-6 months, between 4-12 months, between 6-24 months.

In some embodiments, a method described herein comprises administering an effective amount of the rAAV to a subject, wherein the subject has received, is receiving, or will receive a treatment described herein for increasing tear production. In some embodiments, the treatment described herein for increasing tear production comprises administering one or more doses of a nAChR agonist described herein. In some embodiments, the nAChR agonist is administered via local administration. In some embodiments, the nAChR agonist is administered as an intranasal spray.

In some embodiments, the treatment that increases tear production (e.g., a treatment comprising administering to the subject one or more doses of an nAChR agonist) is administered on a separate schedule from the administration of the rAAV. In some embodiments, the treatment that increases tear production is administered throughout the period between the multiple doses of the rAAV. In some embodiments, the treatment that increases tear production is administered after the first dose of the rAAV. In some of the embodiments described herein, the treatment that increases tear production is administered for at least 1 week, at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least one year, or longer. In some of the embodiments described herein, the treatment that increases tear production is administered for 2-52 weeks, 2-40 weeks, 2-36 weeks, 2-24 weeks, 2-12 weeks, 2-8 weeks, 4-52 weeks, 4-40 weeks, 4-36 weeks, 4-24 weeks, 4-12 weeks, 4-8 weeks, 5-52 weeks, 5-40 weeks, 5-36 weeks, 5-24 weeks, 5-12 weeks, 5-8 weeks, 6-52 weeks, 6-40 weeks, 6-36 weeks, 6-24 weeks, 6-12 weeks, or 6-8 weeks.

In some embodiments, the treatment that increases tear production (e.g., a treatment comprising administering to the subject one or more doses of an nAChR agonist) is administered to the subject one to four times daily after the first day of administration of the rAAV, once a day after the first day of administration of the rAAV, twice a day after the first day of administration of the rAAV, or three times a day after the first day of administration of the rAAV.

In some embodiments, the subject is administered a dosing cycle of an nAChR agonist described herein, wherein the duration between doses of the nAChR agonist is increased over time. For instance, administration of a dose every 4 hours is modified to administration of a dose every 8 or 12 hours.

In some embodiments, the treatment that increases tear production is administered for at least 2 months, at least 3 months, at least 4 months, at least 5 months, at least 6 months, at least 7 months, at least 8 months, at least 9 months, at least 10 months, at least 11 months, or at least one year. In some embodiments, the treatment that increases tear production is administered for 2-52 weeks, 2-40 weeks, 2-36 weeks, 2-24 weeks, 2-12 weeks, 2-8 weeks, 4-52 weeks, 4-40 weeks, 4-36 weeks, 4-24 weeks, 4-12 weeks, 4-8 weeks, 5-52 weeks, 5-40 weeks, 5-36 weeks, 5-24 weeks, 5-12 weeks, 5-8 weeks, 6-52 weeks, 6-40 weeks, 6-36 weeks, 6-24 weeks, 6-12 weeks, or 6-8 weeks.

In some embodiments, the method comprises a first dose and one or more subsequent doses of the rAAV. In some embodiments, the one or more subsequent doses are administered after a period of time after the first dose. This period of time between the first dose and the next subsequent dose is at least 1 hour, at least 2 hours, at least 3 hours, at least 4 hours, at least 5 hours, at least 6 hours, at least 7 hours, or at least 8 hours. The period of time between the first dose and the next subsequent dose is between 1-3 hours, 2-4 hours, 3-6 hours, or 4-8 hours. The period of time between the one or more subsequent doses is at least 1 hour, at least 2 hours, at least 3 hours, at least 4 hours, at least 5 hours, at least 6 hours, at least 7 hours, or at least 8 hours. The period of time between the one or more subsequent doses is between 1-3 hours, 2-4 hours, 3-6 hours, or 4-8 hours.

In some embodiments, the time period between administration of a dose of a rAAV, and a dose of an effective amount of a treatment that increases tear production is less than 5 minutes, between 5-60 minutes, between 30-90 minutes, between 1-3 hours, between 1-8 hours, between 1-12 hours, between 1-24 hours, between 8-12 hours, between 8-24 hours, between 12-24 hours, between 1-3 days, between 1-7 days, between 1-14 days, between 1-28 days, between 3-7 days, between 3-14 days, between 3-28 days, between 7-14 days, or between 7-28 days.

In some embodiments, the method comprises administering a first dose and one or more subsequent doses of the rAAV, and a first dose and one or more subsequent doses of the treatment that increases tear production. The one or more subsequent doses are administered after a period of time after the first dose. This period of time between the first dose and the next subsequent dose is at least 1 hour, at least 2 hours, at least 3 hours, at least 4 hours, at least 5 hours, at least 6 hours, at least 7 hours, or at least 8 hours. The period of time between the first dose and the next subsequent dose is between 1-3 hours, 2-4 hours, 3-6 hours, or 4-8 hours. The period of time between the one or more subsequent doses is at least 1 hour, at least 2 hours, at least 3 hours, at least 4 hours, at least 5 hours, at least 6 hours, at least 7 hours, or at least 8 hours. The period of time between the one or more subsequent doses is between 1-3 hours, 2-4 hours, 3-6 hours, or 4-8 hours.

In some embodiments, the treatment that increases tear production, and the rAAV, are administered to the subject in need thereof in separate dosage forms. In some embodiments, the treatment that increases tear production, and the rAAV, are administered to the subject in need thereof in a combined dosage form.

### Pharmaceutical Compositions and Kits

In some embodiments, the disclosure provides a pharmaceutical composition comprising an rAAV vector described herein. In some embodiments, the pharmaceutical composition comprises an rAAV vector described herein, and a pharmaceutically acceptable carrier, delivery agent, or excipient. In some embodiments, the disclosure provides a pharmaceutical composition comprising a polypeptide comprising a DAO1 enzyme, or a fragment thereof, optionally wherein the polypeptide has an amino acid sequence having at least 90% identity to an amino acid sequence selected from SEQ ID NO: 1 and 2; and b) a pharmaceutically-acceptable carrier suitable for administration to an eye of a human subject. In some embodiments, the disclosure provides a pharmaceutical composition comprising a) a vector comprising a polynucleotide that encodes a polypeptide comprising a DAO1 enzyme, or a fragment thereof, optionally wherein the polypeptide has an amino acid sequence having at least 90% identity to an amino acid sequence selected from SEQ ID NO: 1 and 2; and b) a pharmaceutically-acceptable carrier suitable for administration to an eye of a human subject. In some embodiments, the polypeptide has at least 91, 92, 93, 94, 95, 96, 97, 98, 99, or 100% sequence identity to SEQ ID NO: 1 or SEQ ID NO: 2. In some embodiments, the polypeptide comprises at least 20, 40, 60, 80, 100, 120, 140, 160, 180, 200, 220, 240, 260, 280, 300, 320, 340, 360, 380, 400, 420, 440, 460, 480, or 500 amino acids.

In some embodiments, a pharmaceutical composition may comprise 0.25, 0.50, 0.75, 1.00, 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, or 6.00 μg of a polypeptide comprising DAO1 (or a fragment thereof), or an amount within a range defined by any pair of the foregoing values; and optionally one or more excipients, diluents, and/or carriers. In some aspects, the polypeptide has an amino acid sequence having at least 90% identity to an amino acid sequence selected from SEQ ID NO: 1 and 2. In some embodiments, the polypeptide comprises at least 20, 40, 60, 80, 100, 120, 140, 160, 180, 200, 220, 240, 260, 280, 300, 320, 340, 360, 380, 400, 420, 440, 460, 480, or 500 amino acids. In some embodiments the pharmaceutical composition is formulated for the treatment of an ocular disease, disorder, or condition.

In some embodiments, the disclosure provides use of an rAAV vector or pharmaceutical composition described herein, in the manufacture of a medicament for treatment of an ocular disease, disorder, or condition. In some embodiments, the disclosure provides use of an rAAV vector or pharmaceutical composition herein for use, or adaptable for use, in the treatment of an ocular disease, disorder, or condition.

In some embodiments, the pharmaceutically acceptable carrier comprises phosphate buffered saline. In some embodiments, the pharmaceutical composition is formulated to be compatible with its intended route of administration (e.g., intralacrimal). In some embodiments, the pharmaceutical composition is formulated for administration into the lacrimal gland. In some embodiments, the pharmaceutical composition is formulated for administration onto the ocular surface. Examples of excipients, diluents, and/or carriers suitable for administration to the eye, which can be referred to as pharmaceutically-acceptable carriers, include sterile, pyrogen-free water and sterile, pyrogen-free, buffered saline (e.g., saline buffered using phosphate or other buffers such as HEPES to maintain pH at appropriate physiological levels), isotonic sodium chloride solution, balanced salt solution, emulsions (e.g., oil/water emulsions), and various types of wetting agents.

Any concentration of the vector, such as the rAAV vector, that is suitable to effectively transduce cells of the eye, the lacrimal gland, and/or the nasolacrimal duct can be prepared for contacting cells of the eye, the lacrimal gland, and/or the nasolacrimal duct in vitro or in vivo. For example, the rAAV vector may be formulated at a concentration of 10⁵ vector genomes per mL or more, for example 5×10⁵ vector genomes per mL; 10⁶ vector genomes per mL; 5×10⁶ vector genomes per mL; 10⁷ vector genomes per mL; 5×10⁷ vector genomes per mL; 10⁸ vector genomes per ml; 5×10⁸ vector genomes per mL; 10⁹ vector genomes per mL; 5×10⁹ vector genomes per mL, 10¹⁰ vector genomes per mL, 5×10¹⁰ vector genomes per mL; 10¹¹ vector genomes per mL; 5×10¹¹ vector genomes per mL; 10¹² vector genomes per mL; 5×10¹² vector genomes per mL; 10¹³ vector genomes per mL; 1.5×10¹³ vector genomes per mL; 3×10¹³ vector genomes per mL; 5×10¹³ vector genomes per mL; 7.5×10¹³ vector genomes per mL; 9×10¹³ vector genomes per mL; 10¹⁴ vector genomes per mL; 5×10¹⁴ vector genomes per mL; 10¹⁵ vector genomes per mL; 5×10¹⁵ vector genomes per mL; 10¹⁶ vector genomes per mL; or more, but typically not more than 5×10¹⁶ vector genomes per mL. In some aspects, the rAAV vector may be formulated at a concentration within a range defined by any pair of concentrations described in this paragraph.

In some embodiments, the rAAV vector comprises an AAV capsid and an expression cassette configured to express DAO1, as described herein. Formulations according to the disclosure may comprise any concentration of such rAAV vectors, e.g., at a concentration suitable to effectively transduce cells of the eye, the lacrimal gland, and/or the nasolacrimal duct.

In some embodiments, a formulation may comprise 10⁵; 2.5×10⁵; 5×10⁵; 10⁷; 2.5×10⁷; 5×10⁷; 7.5×10⁷; 10⁸; 2.5×10⁸; 5×10⁸; 7.5×10⁸; 10⁹; 2.5×10⁹; 5×10⁹; 7.5×10⁹; 10¹⁰; 2.5×10¹⁰; 5×10¹⁰; 7.5×10¹⁰; 10¹¹; 2.5×10¹¹; 5×10¹¹; 7.5×10¹¹; 10¹²; 2.5×10¹²; 5×10¹²; 7.5×10¹²; 10¹³; 2.5×10¹³; 5×10¹³; 7.5×10¹³; 1×10¹⁴; 2.5×10¹⁴; 5×10¹⁴; 7.5×10¹⁴; 1×10¹⁵; 1×10¹⁶; or 5×10¹⁶ rAAV vectors per mL, or a concentration of rAAV vectors per mL within a range defined by any pair of concentrations described in this paragraph.

In some embodiments the disclosure provides alternative DAO1 vehicles, such as plasmids configured to express DAO1. Formulations according to the disclosure may comprise any concentration of such plasmids, e.g., at a concentration suitable to effectively transfect cells of the eye, the lacrimal gland, and/or the nasolacrimal duct. In some embodiments, a formulation may comprise 10⁵; 2.5×10⁵; 5×10⁵; 10⁶; 2.5×10⁶; 5×10⁶; 7.5×10⁶; 10⁷; 2.5×10⁷; 5×10⁷; 7.5×10⁷; 10⁸; 2.5×10⁸; 5×10⁸; 7.5×10⁸; 10⁹; 2.5×10⁹; 5×10⁹; 7.5×10⁹; 10¹⁰; 2.5×10¹⁰; 5×10¹⁰; 7.5×10¹⁰; 10¹¹; 2.5×10¹¹; 5×10¹¹; 7.5×10¹¹; 10¹²; 2.5×10¹²; 5×10¹²; 7.5×10¹²; 10¹³; 2.5×10¹³; 5×10¹³; 7.5×10¹³; 10¹⁴; 2.5×10¹⁴; 5×10¹⁴; 7.5×10¹⁴; 10¹⁵; 2.5×10¹⁵; 5×10¹⁵; 7.5×10¹⁵; 10¹⁶; 2.5×10¹⁶; 5×10¹⁶; or 7.5×10¹⁶ plasmids per mL, or a concentration of plasmids per mL within a range defined by any pair of concentrations described in this paragraph.

Similarly, any total number of rAAV vectors suitable to provide appropriate transduction of cells of the eye, the lacrimal gland, and/or the nasolacrimal duct to confer the desired effect or treat the disease can be administered to the mammal or to the primate's eye. In some embodiments, at least 10⁵; 2.5×10⁵; 5×10⁵; 7.5×10⁵; 10⁶; 2.5×10⁶; 5×10⁶; 7.5×10⁶; 10⁷; 2.5×10⁷; 5×10⁷; 7.5×10⁷; 10⁸; 2.5×10⁸; 5×10⁸; 7.5×10⁸; 10⁹; 2.5×10⁹; 5×10⁹; 7.5×10⁹; 10¹⁰; 2.5×10¹⁰; 5×10¹⁰; 7.5×10¹⁰; 10¹¹; 2.5×10¹¹; 5×10¹¹; 7.5×10¹¹; 10¹²; 2.5×10¹²; 5×10¹²; 7.5×10¹²; 10¹³; 2.5×10¹³; 5×10¹³; 7.5×10¹³; 10¹⁴, 2.5×10¹⁴; 5×10¹⁴; 7.5×10¹⁴; 10¹⁵; 2.5×10¹⁵; 5×10¹⁵; or 7.5×10¹⁵; 10¹⁶; 2.5×10¹⁶; 5×10¹⁶; or 7.5×10¹⁶ rAAV vectors, or more, but typically not more than 1×10¹⁵ rAAv vectors are injected per eye. For example, in some embodiments, about 1×10⁹ to about 1×10¹⁰, about 1×10¹⁰ to about 1×10¹¹, about 1×10¹¹ to about 1×10¹², about 1×10¹² to about 1×10¹³, or about 1×10¹³ to about 1×10¹⁵ genome copies of the rAAV vector are administered per eye. In some aspects, the total number of rAAV vectors administered to the eye of the human subject or animal being treated may comprise a concentration within a range defined by any pair of concentrations described in this paragraph. Any suitable number of administrations of the rAAV vectors to the mammal or the primate eye can be made. In some embodiments, the methods comprise a single administration; in other embodiments, multiple administrations are made over time as deemed appropriate by an attending clinician.

In some embodiments, a suitable amount or concentration of rAAV vector (or any other vector described herein) in a therapeutic formulation may be a concentration effective to express from 100 μg/mL to 50 μg/mL of DAO1 in a tear film of a subject subsequent to administration of the composition to the subject. For example, the amount or concentration of the rAAV vector or any other vector described herein (e.g., a plasmid configured to express DAO1), may be one that results in the expression of 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 μg/mL of DAO1 (or a concentration within a range defined by any pair of the foregoing values) in a tear film of a subject. Expression may be measured, e.g., 1, 2, 3, 4, 5, 6, 7, 8, 9, 10, 11, 12, 13, 14, 15, 16, 17, 18, 19, 20, 21, 22, or 23 hours after administration, or after a longer duration of time, such as after 1, 2, 3, 4, or 5 days.

The rAAV vector may be formulated into any suitable unit dosage, including, without limitation, 1×10⁵ vector genomes or more, for example, 1×10⁶, 1×10⁷, 1×10⁸, 1×10⁹, 1×10¹⁰, 1×10¹¹, 1×10¹², 1×10¹³, 1×10¹⁴, or 1×10¹⁵ vector genomes or more, in certain instances, 1×10¹⁴ vector genomes, but usually no more than 4×10¹⁵ vector genomes. In some embodiments, the rAAV vector is formulated into any suitable unit dosage, including, without limitation, 1×10⁵, 1×10⁶, 1×10⁷, 1×10⁸, 1×10⁹, 1×10¹⁰, 1×10¹¹, 1×10¹², 1×10¹³, 1×10¹⁴, or 1×10⁵ vector genomes or more. In some embodiments, the unit dosage is at most about 5×10¹⁵ vector genomes, e.g., 1×10¹⁴ vector genomes or less, for example 1×10¹³, 1×10¹², 1×10¹¹, 1×10¹⁰, or 1×10⁹ vector genomes or less, in certain instances 1×10⁸ vector genomes or less, and typically no less than 1×10⁸ vector genomes. In some embodiments, the unit dosage is at most about 5×10¹⁵ vector genomes, e.g., 1×10¹⁴ vector genomes or less, for example 1×10¹³, 1×10¹², 1×10¹¹, 1×10¹⁰, 1×10⁹, 1×10⁸, or 1×10⁷ vector genomes or less. In some embodiments, the unit dosage is 1×10¹⁰ to 1×10¹¹ vector genomes. In some embodiments, the unit dosage is 1×10¹⁰ to 3×10¹² vector genomes. In some embodiments, the unit dosage is 1×10⁹ to 3×10¹³ vector genomes. In some embodiments, the unit dosage is 1×10⁸ to 3×10¹⁴ vector genomes. In some embodiments, the rAAV vector comprises an AAV capsid and an expression cassette configured to express DAO1 and a unit dosage is formulated at a concentration of at least, at most, exactly, or about 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, or 1×10¹⁶ of the rAAV vector, or at a concentration within a range defined by any pair of concentrations described in this paragraph.

In some embodiments, the disclosure provides a vector comprising a plasmid configured to express DAO1, and a unit dosage is formulated at a concentration of at least, at most, exactly, or about 1×10⁷, 1×10⁸, 1×10⁹, 1×10¹⁰, 1×10¹¹, 1×10¹², 1×10¹³, 1×10¹⁴, or 1×10¹⁵ of the plasmid.

In some embodiments, the unit dosage of pharmaceutical composition may be measured using multiplicity of infection (MOI). By MOI it is meant the ratio, or multiple, of vector or viral genomes of the rAAV vectors provided herein to the cells to which the nucleic acid may be delivered. In some embodiments, the MOI may be 1×10⁶. In some embodiments, the MOI may be 1×10⁵ to 1×10⁷. In some embodiments, the MOI may be 1×10⁴ to 1×10⁸. In some embodiments, recombinant viruses of the disclosure are at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹, 1×10¹⁴, 1×10¹⁵, 1×10¹⁶, 1×10¹⁷, and 1×10¹⁸ MOI. In some embodiments, recombinant viruses of this disclosure are 1×10⁸ to 3×10¹⁴ MOI. In some embodiments, recombinant viruses of the disclosure are at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI.

In some embodiments, the amount of pharmaceutical composition comprises about 1×10⁵ to about 1×10¹⁶ rAAV vectors, 1×10⁸ to about 1×10¹⁵ rAAV vectors, about 1×10⁹ to about 1×10¹⁴ rAAV vectors, about 1×10¹⁰ to about 1×10¹³ rAAV vectors, or about 1×10¹¹ to about 3×10¹² rAAV vectors.

In preparing the subject rAAV compositions, any host cells for producing rAAV vectors may be employed, including, for example, mammalian cells (e.g., 293 cells), insect cells (e.g., SF9 cells), microorganisms and yeast. Host cells can also be packaging cells in which the AAV rep and cap genes are stably maintained in the host cell or producer cells in which the rAAV vector genome is stably maintained and packaged. Exemplary packaging and producer cells are derived from SF-9, 293, A549 or HeLa cells. rAAV vectors are purified and formulated using standard techniques known in the art.

In some embodiments, the disclosure provides for use of an rAAV vector described herein in the manufacture of a medicament. In some embodiments, the disclosure provides for use of an rAAV vector described herein in the manufacture of a medicament for use in a method described herein.

In some embodiments, the disclosure provides a kit comprising an rAAV herein, and instructions for use. In some embodiments, the kit comprises an rAAV herein, and a package insert containing instructions for use of the kit. In some embodiments, the kit comprises an rAAV herein, and a pharmaceutically acceptable carrier, or a pharmaceutical composition comprising the rAAV and instructions for treating or delaying progression of a disease, disorder, or condition described herein in a subject in need thereof.

### Exemplary Embodiments

The disclosure relates to the following embodiments.

Embodiment I-1. A recombinant adeno-associated virus (rAAV) vector comprising an AAV capsid and an expression cassette, the expression cassette comprising a polynucleotide encoding DAO1, operatively linked to a promoter.

Embodiment I-2. The rAAV vector of embodiment I-1, wherein the polynucleotide encodes an amino acid sequence having at least 95% identity to an amino acid sequence selected from SEQ ID NO: 1 and 2.

Embodiment I-3. The rAAV vector of any one of embodiments I-1 to I-2, wherein the polynucleotide comprises a nucleotide sequence having at least 95% identity to a nucleotide sequence selected from SEQ ID NOs: 29-30.

Embodiment I-4. The rAAV vector of any one of embodiments I-1 to I-3, wherein the polynucleotide comprises a nucleotide sequence selected from SEQ ID NOs: 29-30.

Embodiment I-5. The rAAV vector of any one of embodiments I-1 to I-4, wherein the promoter is a CMV promoter comprising the nucleotide sequence set forth in SEQ ID NO: 21 or a CAG promoter comprising the nucleotide sequence set forth in SEQ ID NO: 5.

Embodiment I-6. The rAAV vector of embodiment I-5, wherein the expression cassette comprises the CMV promoter and a CMV enhancer.

Embodiment I-7. The rAAV vector of any one of embodiments I-1 to I-6, wherein the expression cassette comprises a polyadenylation (polyA) sequence.

Embodiment I-8. The rAAV vector of embodiment I-7, wherein the polyA sequence is a BGH polyA sequence.

Embodiment I-9. The rAAV vector of any one of embodiments I-1 to I-8, wherein the expression cassette comprises a Woodchuck Hepatitis Virus Posttranscriptional Regulatory Element (WPRE).

Embodiment I-10. The rAAV vector of any one of embodiments I-1 to I-9, wherein the expression cassette comprises a Kozak sequence.

Embodiment I-11. A composition comprising an rAAV vector, wherein the rAAV vector comprises:
(a) an AAV capsid, and
(b) an expression cassette, wherein the expression cassette comprises a polynucleotide comprising a nucleotide sequence sharing at least 95% identity to a nucleotide sequence selected from SEQ ID NOs: 29-31, and wherein the polynucleotide is linked to a promoter.

Embodiment I-12. The rAAV vector of any one of embodiments I-1 to I-10 or the composition of embodiment I-11, wherein the expression cassette is flanked by two inverted terminal repeats (ITRs).

Embodiment I-13. The rAAV vector or the composition of embodiment I-12, wherein the ITRs are AAV2 ITRs.

Embodiment I-14. The rAAV vector of any one of embodiments I-1 to I-10 and I-12 or the composition of embodiment I-11 or I-12, wherein the expression cassette comprises a sequence that shares at least 90%, 95%, 96%, 97%, 98%, 99%, or 100% identity to SEQ ID NO: 23.

Embodiment I-15. The rAAV vector of any one of embodiments I-1 to I-10 and I-12 to I-14 or the composition of any one of embodiments I-11 to I-14, wherein the AAV capsid comprises a VP3 that shares at least 95%, 98%, or 100% identity with AAV2 VP3 (SEQ ID NO: 8), AAV5 VP3 (SEQ ID NO: 10), AAV8 VP3 (SEQ ID NO: 12), or AAV9 VP3 (SEQ ID NO: 14).

Embodiment I-16. The rAAV vector of any one of embodiments I-1 to I-10 and I-12 to I-15 or the composition of any one of embodiments I-11 to I-15, wherein the AAV capsid comprises a VP3 that shares at least 95%, 98%, or 100% identity with AAV9 VP3 (SEQ ID NO: 14).

Embodiment I-17. A composition comprising an rAAV vector, wherein the rAAV vector comprises:
(a) an AAV2, AAV5, AAV8, or AAV9 capsid, and
(b) an expression cassette, wherein the expression cassette comprises a polynucleotide comprising a nucleotide sequence having at least 95% identity to a nucleotide sequence selected from SEQ ID NOs: 29-31, and wherein the polynucleotide is linked to a promoter.

Embodiment I-18. A composition comprising an rAAV vector, wherein the rAAV vector comprises:
(a) an AAV2, AAV5, AAV8, or AAV9 capsid, and
(b) an expression cassette, wherein the expression cassette comprises a polynucleotide sharing at least 95% identity to SEQ ID NO: 23.

Embodiment I-19. The composition of embodiment I-17 or I-18, wherein the AAV capsid is AAV2.

Embodiment I-20. The composition of embodiment I-17 or I-18, wherein the AAV capsid is AAV5.

Embodiment I-21. The composition of embodiment I-17 or I-18, wherein the AAV capsid is AAV9.

Embodiment I-22. A pharmaceutical composition comprising the rAAV vector of any one of embodiments I-1 to I-10 and I-12 to I-15 or the composition of any one of embodiments I-11 to I-21, and a pharmaceutically acceptable carrier.

Embodiment I-23. The pharmaceutical composition of embodiment I-22, wherein the composition comprises about 1×10⁷ to about 1×10¹⁴ genome copies per milliliter of the rAAV vector.

Embodiment I-24. The pharmaceutical composition of embodiment I-22, wherein the composition comprises about 1×10¹² to about 6.2×10¹² genome copies per milliliter of the rAAV vector.

Embodiment I-25. The pharmaceutical composition of any one of embodiments I-22 to I-24, wherein the composition is formulated for administration into the lacrimal gland.

Embodiment I-26. The pharmaceutical composition of any one of embodiments I-22 to I-25, wherein the composition is formulated for administration onto the ocular surface.

Embodiment I-27. The pharmaceutical composition of any one of embodiments I-22 to I-26, wherein the composition is formulated for use, or adaptable for use, in the treatment of an ocular disease, disorder, or condition.

Embodiment I-28. A method of treating a condition in a subject in need thereof, the method comprising administering an effective amount of the pharmaceutical composition of any one of embodiments I-22 to I-27 to the eye of the subject.

Embodiment I-29. The method of embodiment I-28, wherein the pharmaceutical composition is delivered to an ocular secretory gland of the subject.

Embodiment I-30. The method of embodiment I-28 or I-29, wherein the pharmaceutical composition is delivered to the lacrimal gland.

Embodiment I-31. The method of embodiment I-30, wherein cells within the lacrimal gland are transduced by the rAAV vector.

Embodiment I-32. The method of embodiment I-31, wherein the transduced cells within the lacrimal gland express an effective amount of DAO1 into the tear film and optionally onto the ocular surface of the subject.

Embodiment I-33. The method of any one of embodiments I-28 to I-32, wherein the pharmaceutical composition is delivered to an accessory lacrimal gland.

Embodiment I-34. The method of any one of embodiments I-28 to I-33, wherein about 1×10⁹ to about 1×10¹⁰, about 1×10¹⁰ to about 1×10¹¹, about 1×10¹¹ to about 1×10¹², about 1×10¹² to about 1×10¹³, or about 1×10¹³ to about 1×10¹⁵ genome copies of the rAAV vector are administered.

Embodiment I-35. The method of any one of embodiments I-28 to I-34, wherein the condition is associated with increased histamine production and/or increased histamine signaling.

Embodiment I-36. The method of any one of embodiments I-28 to I-35, wherein the condition is an inflammatory condition, optionally an inflammatory condition of the eye.

Embodiment I-37. The method of any one of embodiments I-28 to I-36, wherein the condition is an autoimmune condition.

Embodiment I-38. The method of any one of embodiments I-28 to I-36, wherein the condition is an allergic condition.

Embodiment I-39. The method of embodiment I-38, wherein the condition is an allergic reaction to a therapeutic agent.

Embodiment I-40. The method of embodiment I-38, wherein the condition is an allergic reaction to a microbial agent.

Embodiment I-41. The method of any one of embodiments I-28 to I-36, wherein the condition comprises vernal keratoconjunctivitis.

Embodiment I-42. The method of any one of embodiments I-28 to I-36, wherein the condition comprises atopic keratoconjunctivitis.

Embodiment I-43. The method of any one of embodiments I-28 to I-36, wherein the condition comprises seasonal or perennial allergic conjunctivitis.

Embodiment I-44. The method of any one of embodiments I-28 to I-43, wherein the administration results in expression of DAO1 in the cells of the lacrimal gland and/or an accessory lacrimal gland.

Embodiment I-45. The method of any one of embodiments I-28 to I-44, wherein the administration results in secretion of DAO1 into the tear film.

Embodiment I-46. The method of embodiment I-45, wherein secretion of DAO1 into the tear film is stimulated by a cholinergic agonist.

Embodiment I-47. The method of any one of embodiments I-28 to I-46, wherein the administration results in an improvement of one or more symptoms of the condition.

Embodiment I-48. The method of embodiment I-47, wherein the symptom is selected from the group consisting of itching, swelling, tearing, and redness.

Embodiment I-49. The method of any one of embodiments I-28 to I-46, wherein the administration results in an improvement of 0.5 points, 1 point, 1.5 points, 2 points, 2.5 points, 3 point, 3.5 points, or 4 points on the Conjunctival Itching Grading Scale.

Embodiment I-50. The method of any one of embodiments I-28 to I-46, wherein the administration results in an improvement of 1 point, 2 points, 3 point, or 4 points on the Conjunctival Redness Assessment Grading Scale.

Embodiment I-51. The method of any one of embodiments I-28 to I-46, wherein the administration results in an improvement of about 10% to about 20%, about 20% to about 30%, about 30% to about 40%, about 40% to about 50%, about 50% to about 60%, about 60% to about 70%, about 70% to about 80%, about 80% to about 90%, or more than about 95% on the Schirmer Test.

Embodiment I-52. The method of any one of embodiments I-28 to I-46, wherein the administration results in an improvement of about 10% to about 20%, about 20% to about 30%, about 30% to about 40%, about 40% to about 50%, about 50% to about 60%, about 60% to about 70%, about 70% to about 80%, about 80% to about 90%, or more than about 95% in at least one area on the Corneal Staining Grading Scale.

Embodiment I-53. The method of any one of embodiments I-47 to I-52, wherein the improvement is measured about 1 months, about 2 months, about 3 months, about 4 months, about 5 months, about 6 months, about 9 months, or about 12 months after the administration.

Embodiment I-54. The method of any one of embodiments I-47 to I-52, wherein the improvement persists for at least 3 months, at least 6 months, at least 9 months, at least 12 months, at least 15 months, at least 18 months, at least 24 months, at least 3 years, at least 4 years, or at least 5 years after the administration.

Embodiment I-55. The method of any one of embodiments I-28 to I-54, further comprising administering one or more additional therapeutic agents to the subject.

Embodiment I-56. The method of embodiment I-55, wherein the one or more additional therapeutic agents increases tear production.

Embodiment I-57. The method of embodiment I-56, wherein the one or more additional therapeutic agents that increase tear production is administered via local nasal administration.

Embodiment I-58. The method of embodiment I-57, wherein the local nasal administration is via an intranasal spray.

Embodiment I-59. The method of any one of embodiments I-56 to I-58, wherein the one or more additional therapeutic agents that increase tear production comprises a nicotinic acetylcholine receptor (nAChR) agonist, or a pharmaceutically acceptable salt thereof.

Embodiment I-60. The method of embodiment I-59, wherein the nAChR agonist is a full agonist of a nAChR subtypes selected from alpha4beta2, alpha3beta4, alpha3alpha5beta4, alpha4alpha6beta2, and a combination thereof.

Embodiment I-61. The method of embodiment I-59 or I-60, wherein the nAChR agonist is varenicline, or a pharmaceutically acceptable salt thereof.

Embodiment I-62. The method of embodiment I-59 or I-60, the nAChR agonist is (R)-5-((E)-2-pyrrolidin-3-ylvinyl)pyrimidine, or a pharmaceutically acceptable salt thereof.

Embodiment I-63. The method of any one of embodiments I-56 to I-62, wherein the one or more additional therapeutic agents that increases tear production is administered prior to or following the administering of the pharmaceutical composition.

Embodiment I-64. The method of embodiment I-56 to I-63, wherein the one or more additional therapeutic agents that increases tear production is administered at about 1 week following the administering of the pharmaceutical composition.

Embodiment I-65. The method of any one of embodiment I-56 to I-64, resulting in expression of DAO1 in the tear film and/or cornea of the subject following administration of the pharmaceutical composition.

Embodiment I-66. The method of embodiment I-65, wherein the expression of DAO1 in the tear film and/or cornea is increased in a predetermined amount of time compared to an administration of the pharmaceutical composition without the one or more additional therapeutic agents that increases tear production.

Embodiment I-67. The method of embodiment I-66, wherein the predetermined amount of time is about 5 minutes.

Embodiment I-68. The method of embodiment I-66, wherein the predetermined amount of time is about 1 hour.

Embodiment I-69. The method of any one of embodiments I-28 to I-68, wherein the subject is human.

Embodiment I-70. The pharmaceutical composition of any one of embodiments I-22 to I-27 for use in a method of treating a condition in a subject in need thereof comprising administering an effective amount of the pharmaceutical composition to the eye of the subject.

Embodiment I-71. The pharmaceutical composition of any one of embodiments I-22 to I-27 for use in the manufacture of a medicament for treating a condition in a subject in need thereof.

Embodiment I-72. A kit comprising a pharmaceutical composition of rAAV vector of any one of embodiments I-1 to I-10 and I-12 to I-15 or the composition of any one of embodiments I-11 to I-21, and a pharmaceutically acceptable carrier, and instructions for use in treating a condition in a subject, comprising administering the pharmaceutical composition to the eye of the subject.

Embodiment I-73. A kit comprising a pharmaceutical composition of rAAV vector of any one of embodiments I-1 to I-10 and I-12 to I-15 or the composition of any one of embodiments I-11 to I-21, and a pharmaceutically acceptable carrier, and instructions for use in treating a condition associated with histamine production and/or increased histamine signaling in a subject, comprising administering the pharmaceutical composition to the eye of the subject.

Embodiment I-74. A kit comprising a pharmaceutical composition of rAAV vector of any one of embodiments I-1 to I-10 and I-12 to I-15 or the composition of any one of embodiments I-11 to I-21, and a pharmaceutically acceptable carrier, and instructions for use in treating an autoimmune condition in a subject, comprising administering the pharmaceutical composition to the eye of the subject.

Embodiment I-75. A kit comprising a pharmaceutical composition of rAAV vector of any one of embodiments I-1 to I-10 and I-12 to I-15 or the composition of any one of embodiments I-11 to I-21, and a pharmaceutically acceptable carrier, and instructions for use in treating an allergy condition in a subject, comprising administering the pharmaceutical composition to the eye of the subject.

Embodiment I-76. The kit of any one of embodiments I-72 to I-75, wherein the subject is receiving, has received, or subsequently receives one or more additional therapeutic agents that increases tear production.

Embodiment I-77. The kit of embodiment I-76, wherein the one or more additional therapeutic agents that increases tear production comprises a nicotinic acetylcholine receptor (nAChR) agonist, or a pharmaceutically acceptable salt thereof.

Embodiment I-78. The kit of embodiment I-76 or I-77, wherein the one or more additional therapeutic agents that increases tear production comprises varenicline, or a pharmaceutically acceptable salt thereof.

Embodiment I-80. The kit of embodiment I-76 or I-77, wherein the one or more additional therapeutic agents that increases tear production comprises (R)-5-((E)-2-pyrrolidin-3-ylvinyl)pyrimidine, or a pharmaceutically acceptable salt thereof.

Embodiment I-81. The kit of any one of embodiments I-76 to I-80, wherein the subject receives the one or more additional therapeutic agents after administration of the pharmaceutical composition.

Embodiment I-82. A pharmaceutical composition, comprising a) a polypeptide comprising a diamine oxidase (“DAO1”) enzyme, or a fragment thereof, optionally wherein the polypeptide has an amino acid sequence having at least 90% identity to an amino acid sequence selected from SEQ ID NO: 1 and 2; and b) a pharmaceutically-acceptable carrier suitable for administration to an eye of a human subject.

Embodiment I-83. A pharmaceutical composition, comprising a) a vector comprising a polynucleotide that encodes a polypeptide comprising a diamine oxidase (“DAO1”) enzyme, or a fragment thereof, optionally wherein the polypeptide has an amino acid sequence having at least 90% identity to an amino acid sequence selected from SEQ ID NO: 1 and 2; and b) a pharmaceutically-acceptable carrier suitable for administration to an eye of a human subject.

Embodiment I-84. The pharmaceutical composition of embodiments I-82 or I-83, wherein the pharmaceutically-acceptable carrier comprises water; sterile water; pyrogen-free water; phosphate-buffered saline; HEPES-buffered saline; an isotonic sodium chloride solution; a balanced salt solution; a wetting agent; a surfactant; a tonicity agent; a pH modifier; a viscosity-modifying agent; a buffering agent; a disaccharide, optionally, sucrose or trehalose; a cellulose and/or a derivate thereof; an amino acid, optionally histidine; or any combination thereof.

Embodiment I-85. The pharmaceutical composition of any one of embodiments I-82 to I-84, wherein the polypeptide has at least 91, 92, 93, 94, 95, 96, 97, 98, 99, or 100% sequence identity to SEQ ID NO: 1 or SEQ ID NO: 2.

Embodiment I-86. The pharmaceutical composition of any one of embodiments I-82 to I-85, wherein the polypeptide comprises at least 20, 40, 60, 80, 100, 120, 140, 160, 180, 200, 220, 240, 260, 280, 300, 320, 340, 360, 380, 400, 420, 440, 460, 480, or 500 amino acids.

Embodiment I-87. The pharmaceutical composition of any one of embodiments I-82 to I-86, wherein the formulation is a liquid formulated for application to an ocular surface, or into an ocular surface, or for intralacrimal injection, of the eye of the human subject.

Embodiment I-88. The pharmaceutical composition of any one of embodiments I-82 to I-87, comprising the vector, wherein the vector is present in the composition in an amount effective to express from 100 μg/mL to 50 μg/mL of the polypeptide in a tear film of a subject subsequent to administration of the composition to the subject.

Embodiment I-89. The pharmaceutical composition of any one of embodiments I-82 to I-88, comprising the vector, wherein the polynucleotide is operably linked to a promotor.

Embodiment I-90. The pharmaceutical composition of any one of embodiments I-82 to I-89, comprising the vector, wherein the vector is engineered to constitutively express the polypeptide having an amino acid sequence having at least 90% identity to an amino acid sequence selected from SEQ ID NO: 1 and 2.

Embodiment I-91. The pharmaceutical composition of any one of embodiments I-82 to I-90, comprising the vector, wherein the vector comprises a virus, optionally an adenoviral vector or a lentiviral vector; a plasmid; an episome; or an artificial chromosome; and optionally comprises one or more lipids, polycations, DNA-carrier proteins, histones, pseudocapsids, chimeric proteins, or endocytosis receptor proteins.

Embodiment I-92. The pharmaceutical composition of any one of embodiments I-82 to I-87, comprising the polypeptide, wherein the polypeptide is present in the pharmaceutical composition at a concentration of 100 μg/mL to 50 μg/mL.

Embodiment I-93. The pharmaceutical composition of any one of embodiments I-82 to I-87, or I-93, comprising the polypeptide, wherein the polypeptide is present in the pharmaceutical composition in an amount of 500 ng to 5 ug.

Embodiment I-94. The pharmaceutical composition of any one of embodiments I-82 to I-87, I-92, or I-93, comprising the polypeptide, wherein the polypeptide is present in the pharmaceutical composition in a unit dose amount.

Embodiment I-95. A method of treating an ocular disease, disorder, or condition in a subject in need thereof, the method comprising administering an effective amount of the pharmaceutical composition of any one of embodiments I-82 to I-94 to an eye of the subject.

Embodiment I-96. The method of embodiment I-95, wherein the ocular disease, disorder, or condition is associated with increased histamine production and/or increased histamine signaling.

Embodiment I-97. The method of embodiments I-95 or I-96, wherein the condition is an inflammatory condition, optionally an inflammatory condition of the eye.

Embodiment I-98. The method of any one of embodiments I-95 to I-97, wherein the condition is an autoimmune condition.

Embodiment I-99. The method of any one of embodiments I-95 to I-98, wherein the condition is an allergic condition.

Embodiment I-100. The method of any one of embodiments I-95 to I-99, wherein the condition is an allergic reaction to a therapeutic agent.

Embodiment I-101. The method of any one of embodiments I-95 to I-100, wherein the condition is an allergic reaction to a microbial agent.

Embodiment I-102. The method of any one of embodiments I-95 to I-101, wherein the condition comprises vernal keratoconjunctivitis.

Embodiment I-103. The method of any one of embodiments I-95 to I-102, wherein the condition comprises atopic keratoconjunctivitis.

Embodiment I-104. The method of any one of embodiments I-95 to I-103, wherein the condition comprises seasonal or perennial allergic conjunctivitis.

Embodiment I-105. The method of any one of embodiments I-95 to I-104, wherein the administration results in expression of a functional diamine oxidase in one or more cells of a lacrimal gland and/or an accessory lacrimal gland of the subject.

Embodiment I-106. The method of any one of embodiments I-95 to I-105, wherein the administration results in secretion of a functional diamine oxidase into a tear film of the subject.

Embodiment I-107. The method of any one of embodiments I-95 to I-106, wherein secretion of the functional diamine oxidase into the tear film is stimulated by a cholinergic agonist.

Embodiment I-108. The method of embodiment I-107, wherein the administration is to an ocular surface, or into an ocular surface of the subject, and/or to a lacrimal gland of the subject.

Embodiment I-109. The method of any one of embodiments I-95 to I-108, wherein the symptom is selected from the group consisting of itching, swelling, tearing, and redness.

Embodiment I-110. The method of any one of embodiments I-95 to I-109, wherein the administration results in an improvement of 0.5 points, 1 point, 1.5 points, 2 points, 2.5 points, 3 point, 3.5 points, or 4 points on the Conjunctival Itching Grading Scale.

Embodiment I-111. The method of any one of embodiments I-95 to I-110, wherein the subject is a human subject.

Embodiment I-112. A kit comprising the pharmaceutical composition of any one of embodiments I-82 to I-94 and instructions for use in treating a condition in a human subject, comprising administering the pharmaceutical composition to an eye of the human subject.

Embodiment I-113. The pharmaceutical composition of any one of embodiments I-82 to I-94 for use in the manufacture of a medicament for treating a condition in a human subject in need thereof.

### EXAMPLES

The following specific examples are to be construed as merely illustrative, and not limitative of the remainder of the disclosure in any way whatsoever.

### Example 1: In Vitro Transfection of 293T Cells with an AAV Transfer Plasmid Encoding DAO1

A study was performed to assess expression and activity of DAO1 polypeptide following in vitro transfection of 293T cells with the AAV transfer plasmid depicted in FIG. 1A (referred to as “AAV.DAO1”). The AAV transfer plasmid contained a 5′ and 3′ AAV2 ITR (SEQ ID NOs: 16 and 17 respectively). Between the 5′ and 3′ ITR was a cDNA encoding a human DAO1 polypeptide and cDNA for elements to provide an optimal expression level of DAO1 polypeptide. A schematic depicting the construct is shown in FIG. 1B. The encoded human DAO1 polypeptide has the amino acid sequence set forth in SEQ ID NO: 2. The cDNA encoding the human DAO1 polypeptide was codon optimized for improved expression in human cells and has the nucleotide sequence set forth in SEQ ID NO: 29. The nucleotide sequence from 5′ITR to 3′ITR is set forth in SEQ ID NO: 23.

Plasmid expansion was performed by adding 10 ng of AAV.DAO1 plasmid to 50 μl of New England Biolabs (NEB) Stable Competent E. coli High Efficiency (NEB C3040) following the manufacturer's rapid transformation protocol. A single bacterial colony was selected and utilized to inoculate 5 mL Luria Broth (LB) using kanamycin (50 ug/mL) as a positive selection for relevant colony clones. The starter culture was incubated for 16 hours at 30° C., and then used to inoculate 250 mL of LB/kanamycin at a 1/500 dilution. This culture was incubated for 16 hours at 30° C., then centrifuged for 20 minutes at 4,000×g to pellet the bacteria. The Qiagen Hi-Speed Maxi Kit (Qiagen 12662) was used to purify the plasmid DNA per the manufacturer's protocol. The plasmid was verified by restriction enzyme digestion and agarose gel electrophoresis based upon fragment sizes.

293T cells were plated in 6 well plates at 6.5×10⁵ cells per well and transfected with 2.5 μg of AAV.DAO1 plasmid DNA utilizing Lipofectamine 3000 per the manufacturer's protocol. Control cells were transfected with an empty vector. The cells were grown out and then plated at 5.5×10⁵ on 35 mm glass bottom tissue culture plates.

### Immunofluorescence

Immunofluorescence imaging was performed to detect DAO1 expression in transfected cells.

293T cells were plated at 5.5×10⁵ and PANC-1 cells were plated at 4×10⁵ on Poly-L-Lysine (VWR 103701-192) coated 33 mm glass bottom TC dish (MatTek P35G-0-10-C). Twelve to twenty-four (12-24) hours after plating the cells were transfected according to the Lipofectamine3000 (ThermoFisher L300015) transfection protocol using 3.75 μl Lipofectamine 3000 and 2.5 ug pAAV-DAO plasmid per well. 24 hours post transfection the media was aspirated, and the cells were washed twice with pre-warmed PBS (37° C.). 500 μl of 4% paraformaldehyde was added and incubated for 10 minutes at room temperature. The cells were washed 2 times with PBS. 500 μl of blocking buffer was added and the cells were incubated for 45 minutes at room temperature. Blocking buffer consisted of PBS, 0.1% Triton X and 1% bovine serum albumin (BSA). The cells were stained with a primary antibody to diamine oxidase and a fluorescently-tagged secondary antibody. The cells were counterstained with DAPI and then imaged on a fluorescent microscope. Specifically, primary antibodies were diluted in blocking buffer then 200 μl of the diluted mixture was added to the central glass portion of the dish and incubated for 2 hours at 37° C. Primary antibody concentrations were optimized experimentally, and a 1:50 dilution was used. The cells were washed 3 times for 15 minutes with PBS. Fluorescently labeled secondary antibodies and DAPI were diluted 1:400 in PBS, added to the glass portion of the dish, and incubated at room temperature for 1 hour (protected from light). The cells were then washed 3 more times for 15 minutes with PBS. The cells were then imaged on a Keyence BZ-X series microscope.

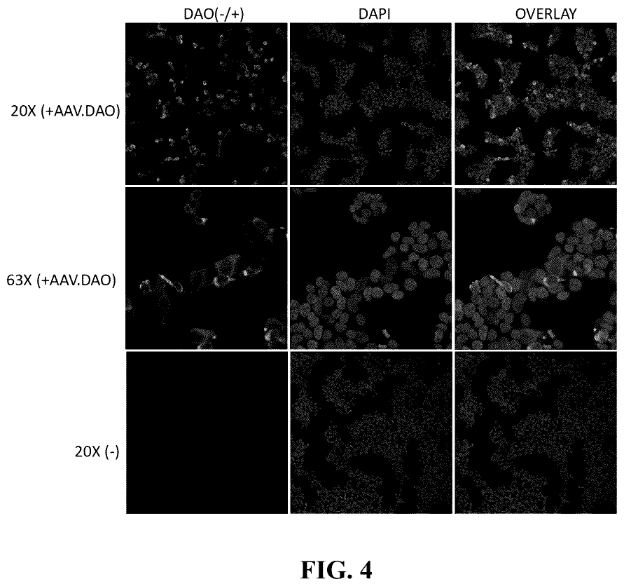

As shown in FIGS. 4A-4B, expression of DAO1 protein (as indicated by the bright and punctate signal) was measured in 293T cells transfected with the AAV transfer plasmid, but not in control cells. As shown in FIGS. 4C-4D, expression of DAO1 protein was further detected in PANC-1 cells transiently transfected with AAV.DAO plasmid.

### Western Blot

Western blot was performed to detect DAO1 expression in conditioned media from AAV.DAO1-transfected cells as compared to control cells, with detection of DAO1 performed using a rabbit anti-DAO1 antibody and labeled anti-rabbit secondary antibody. For conditioned media collection, cell media was aspirated at 24 hours post-transfection and washed twice with 2 mL pre-warmed Opti-MEM (ThermoFisher 31985-070). 1.4 mL of Opti-MEM was then added and conditioned media was collected at 2,4 and 6 hour intervals. Immediately following collection, 1× protease inhibitor was added and the conditioned media was placed on ice for 5 min, centrifuged at 4° C. 12,000×g for 5 minutes to remove culture debris. The conditioned media supernatant was then concentrated 40× using an Amicon Ultra-2 Centrifugal Filter Unit (Millipore UFC201024) per manufacturer's protocol and stored at −20° C.

Western blot was also performed using whole cell extracts. Whole cell extracts were prepared by aspirating cell media at 24 or 48 hours post-transfection. The cells were washed twice with 2 mL of 4° C. PBS, then lysed using 300 μl RIPA buffer (Sigma R0278) containing protease/phosphatase inhibitor (Cell Signaling 5872S) diluted to 1× from 100× stock. The whole cell extract was then centrifuged at 12,000×g for 5 min at 4° C. to remove any cellular debris and then supernatant was aliquoted and stored at −20° C. The collected whole cell extract (per above methodology) was quantified using a micro-bicinchoninic acid assay (BCA) protein assay kit (Thermo Fisher 23235) per manufacturer's recommendations. Sample concentrations were normalized by dilution with double distilled water (ddH₂0) and Laemmli buffer. The extracts were then heated to 100° C. for 10 minutes. Samples were electrophoresed on 10% SDS-PAGE gels and then transferred to a PVDF membrane using the Invitrogen iBlot 2 Dry Blotting System. The membrane was blocked using 5% Blotting-Grade Blocker (BioRad 1706404) for 1 hour at room temperature on a rotating platform. Primary antibodies were diluted in 5% blocking buffer with 0.1% Tween 20 and incubated at 4° C. overnight on a rocking platform. Optimized primary antibody concentrations were determined experimentally. The membrane was washed in PBS with 0.05% Tween 20 for 10 minutes on a rotating platform. The washing step was repeated 4 times. HRP conjugated secondary antibodies were diluted (1:10,000) in 5% blocker with 0.1% Tween 20 and incubated at room temperature on a rotating platform for 1 hour. The previous washing steps were repeated with the final wash being PBS alone. The membrane was incubated for 1 minute in SuperSignal West Pico Plus (Thermo Fisher 34577) and chemiluminescent detection was done on a BioRad ChemiDoc MP.

As a control, Western blot was also performed using a human DAO1 recombinant protein (40 ng) and a mouse anti-his antibody. Western blot of GAPDH (30 μg/well) served as a loading control.

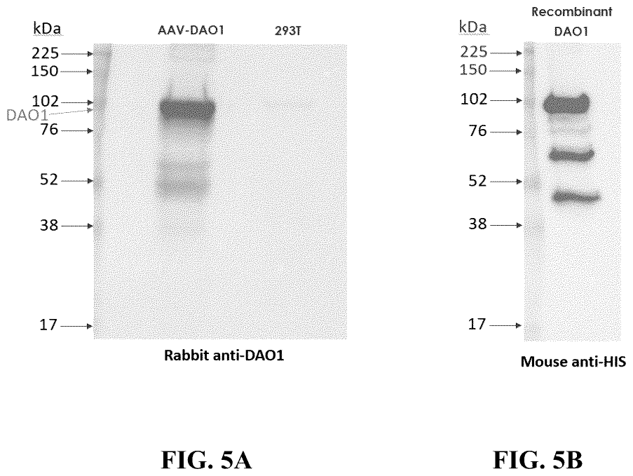

As shown in FIG. 5A, DAO1 polypeptide was observed in media obtained from transfected cells, but not control cells. The DAO1 polypeptide had the same molecular weight as the human DAO1 recombinant protein that was used as a positive control (FIG. 5B). As shown in FIGS. 5C-5D, DAO1 of the expected size (kDa) was detected in whole cell extracts obtained from transfected 293T cells. Recombinant DAO1 served as a positive control (FIG. 5C) and GAPDH as a loading control (FIG. 5D).

Additionally, DAO1 expression was measured in conditioned media at 2, 4, and 6 hours after a fresh media change. As shown in FIG. 5E, a significant level of DAO1 was detected in as little as 2 hours following media change.

Together these data demonstrate expression and secretion of DAO1 into the cell culture media.

### DAO1 Enzymatic Activity Assay

To assess the functional activity of the expressed DAO1 protein, an activity assay was conducted. The principle of the assay is a substrate is provided that in the presence of active DAO1 results in a reaction where hydrogen peroxide (H₂O₂) is released. The H₂O₂ released by the cleavage of a substrate reacts with a second substrate and produces a fluorescent signal that excites at 535 nm and emits at 587 nm. The amount of fluorescence recorded at 587 nm indicates the amount of enzymatic activity.

The experimental approach involved plating 293T cells in a 6-well plate format and transfecting with AAV.DAO. Negative control cells were non-transfected. Twenty four hours post-transfection, the media was removed and replenished with 2 mL/well maintenance media. Twenty four later the conditioned media and whole cell extract (WCE) were collected. Upon collection, protease inhibitor was added, and the conditioned media/WCE was placed on ice for 5 min and centrifuged at 4° C. 12,000×g for 5 minutes to remove debris. The media/WCE was aliquoted and stored at −20° C. A micro-BCA assay was conducted to quantify protein levels of the WCE. Standards were prepared using 10 mM H₂O₂ supplied with the Diamine Oxidase Activity Assay Kit (Sigma MAK351). The serially diluted standards, undiluted AAV.DAO conditioned media, 100 ng WCE and control samples were added to the 96-well reaction plate. A reaction mix was prepared using DAO Assay Buffer, substrate, enzyme mix and probe. The reaction plate was then incubated at 37° C. for 90 minutes and kinetic measurements were taken at 30, 60 and 90 minutes post-incubation. Measurements were taken at 535 nm excitation wavelength and a 587 nm emission wavelength using a Molecular Devices ID3 plate reader. DAO1 activity was measured in nmol H₂O₂ generated in 90 minutes (experiment 1) or pmol/min/μl (experiment 2 using kinetic measurements).

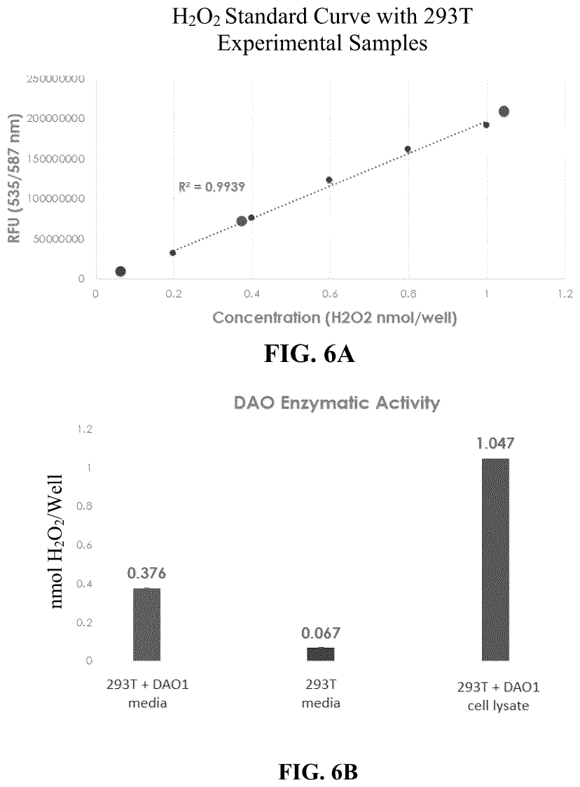

As shown in FIG. 6A, a calibration curve was generated by measuring fluorescent signal (535 nm/587 nm) in the presence of increasing concentrations of H₂O₂. The assay was used to measure the activity of DAO1 in conditioned media obtained from AAV.DAO1-transfected 293T cells or cell lysates combined with the conditioned media and compared to activity measured in conditioned media obtained from non-transfected 293T cells. As shown in FIG. 6B, substantially higher H₂O₂ was generated for conditioned media obtained from AAV.DAO1-transfected 293T cells alone or combined with cell lysates as compared to conditioned mediate obtained from non-transfected 293T cells. As shown in FIG. 6C, kinetic measurements of DAO1 activity in conditioned media of transfected 293T cells at 30, 60, and 90 minutes demonstrates target modulation capability and functional activity of DAO1 expressed by AAV-DAO.

This demonstrates that the DAO1 protein present in the cells and expressed into the cell culture media is able to cleave the given substrate and demonstrates functional enzymatic activity.

### Example 2: Intra-Lacrimal Gland Gene Therapy Using rAAV Vector

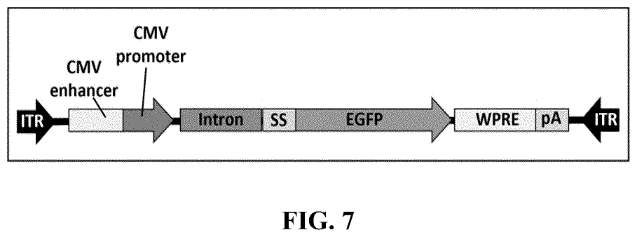

This example shows a 9-day pilot study of single dose rAAV vectors administered as an intra-lacrimal gland injection followed by a single dose of varenicline administered as an intranasal dose to Dutch-Belted rabbits. It evaluates the effectiveness and tolerability of a panel of rAAV vector embodiments administered one time via injection to the lacrimal gland. Each rAAV vector composition in the panel is tested at two concentrations (1×10¹² GC/mL and 6.2×10¹² GC/mL). The panel of rAAV vectors include embodiments with capsid proteins having AAV2, AAV5, AAV8, and AAV9 serotypes. The expression cassette delivered by the rAAV vector encoded an enhanced green fluorescent protein (eGFP) transgene that is operatively linked to a CMV promoter (FIG. 7). On day 9 following injection of the rAAV vector, the animal is given an intranasal dose of varenicline. The varenicline induces tear production in the animals such that eGFP delivered to the lacrimal by the rAAV vector and expressed under control of the CMV promoter in cells of the lacrimal gland will be secreted into the tear film and onto the ocular surface of the animal. Two main objectives will be achieved using this approach:
(1) Test the feasibility of capsid protein serotypes AAV2, AAV5, AAV8, and AAV9 to deliver a transgene to cells in the lacrimal gland resulting in measurable CMV promoter-driven expression of the transgene within the cells.
(2) Assess the feasibility of increasing the relative amount of the transgene encoded by the by the expression cassette into the tear film and onto the ocular surface of the animal.

Animal studies were carried out in the Charles River Laboratories (CRL) facilities by CRL staff scientific personnel.

### Animal Test System, Husbandry, and In-life Monitoring

The animals used in this study were male Dutch-Belted rabbits between the ages of 4 to 5 months and weighed between 1.3 to 2.3 kg. Animals were acclimated for 10 days prior to the start of treatment. Each animal was housed individually and cared for using standard caregiving protocols including regular environmental conditions, feeding schedules, and veterinary care.

rAAV Vector Compositions and Formulations

In this study, the panel of compositions comprising rAAV vectors containing an expression cassette encoding an eGFP transgene operatively linked to a CMV promoter are provided for intralacrimal injection under the conditions in Table 6. Each composition contains an rAAV vector with a different AAV capsid protein serotype. The compositions are labeled as OC-100a-d, each corresponding to a different AAV capsid protein serotype. Dose formulations for intralacrimal injection were prepared using clean procedures at the target concentrations described below in Table 8 by diluting with phosphate buffered saline solution.

TABLE 6
Summary of rAAV Compositions

| | rAAV | rAAV | rAAV | rAAV |
|---|---|---|---|---|
| | Composition 1 | Composition 2 | Composition 3 | Composition 4 |
| | (OC-100a) | (OC-100b) | (OC-100c) | (OC-100d) |
| AAV Capsid | AAV2 | AAV5 | AAV8 | AAV9 |
| Serotype | | | | |
| Physical | Clear Liquid | Clear Liquid | Clear Liquid | Clear Liquid |
| Description | | | | |
| Concentration | 1.5 × 10¹³ | 1.3 × 10¹³ | 1.1 × 10¹³ | 4 × 10¹³ |
| | GC/mL | GC/mL | GC/mL | GC/mL |
| Storage | −80° C. | −80° C. | −80° C. | −80° C. |
| Conditions | | | | |

Intralacrimal Injection of rAAV Compositions

Animals were dosed via intralacrimal injection on day 1 of the study. A summary of the formulation concentration for each composition tested, dose volume, dose frequency, and number of animals and lacrimal glands is found in Table 7. Prior to the injection, animals were anesthetized by intramuscular injection of dexmedetomidine (0.25 mg/kg) followed by an isoflurane/oxygen mix through a mask to maintain anesthesia, if necessary. A topical antibiotic was applied to each eye after dose administration. On day 9 of the study, animals were given intranasal administration of varenicline tartrate (50 μL per nostril of 1.2 mg/mL varenicline) to induce tear production.

TABLE 7
Summary of Experimental Design

| | | Formulation | | | | Number of |
|---|---|---|---|---|---|---|
| | | Concentration | | | Number | Lacrimal |
| Group | rAAV | (genome copies | Dose Volume | Dose | of | Glands |
| No. | Composition | (GC)/mL) | (μL/gland/dose) | Frequency | Animals | Injected |
| 1 | OC-100a | 1 × 10¹² | 100 | once | 1 | 2 |
| 2 | OC-100a | 6.2 × 10¹² | 100 | once | 2 | 4 |
| 3 | OC-100b | 1 × 10¹² | 100 | once | 1 | 2 |
| 4 | OC-100b | 6.2 × 10¹² | 100 | once | 2 | 4 |
| 5 | OC-100c | 1 × 10¹² | 100 | once | 1 | 2 |
| 6 | OC-100c | 6.2 × 10¹² | 100 | once | 2 | 4 |
| 7 | OC-100d | 1 × 10¹² | 100 | once | 1 | 2 |
| 8 | OC-100d | 6.2 × 10¹² | 100 | once | 2 | 4 |

### Bioanalysis

Blood was collected on Day 1 before dosing, and again on Day 8 and 9 (after the intranasal dosing at approximately 1 hour post dose) from an auricular vessel from all animals. Blood samples were placed on ice until plasma is separated by centrifugation. Plasma samples were separated into 250 μL aliquots and frozen at −80° C. for subsequent analysis.

A Schirmer tear test were performed to collect eye moisture from the animals on Day 8 and 9. Test strips were placed inside the lower eyelid for approximately 1 minute. The paper was removed and placed into separate tubes and frozen at −80° C. for subsequent analysis.

Plasma samples and Schirmer's test strips were analyzed for concentration of eGFP and eGFP mRNA concentration using a validated procedure at Syneos analytical laboratories.

### Immunohistochemistry of Lacrimal Gland Tissue

Animals were euthanized on day 9 following collection of blood and eye moisture by intravenous injection of sodium pentobarbital. Five sagittal sections of the left eye and sections of the left and right lacrimal glands were prepared for immunohistochemistry (IHC) according to lab standard operating procedure. Lacrimal gland IHC samples were stained for eGFP and subject to microscopic evaluation.

### Results

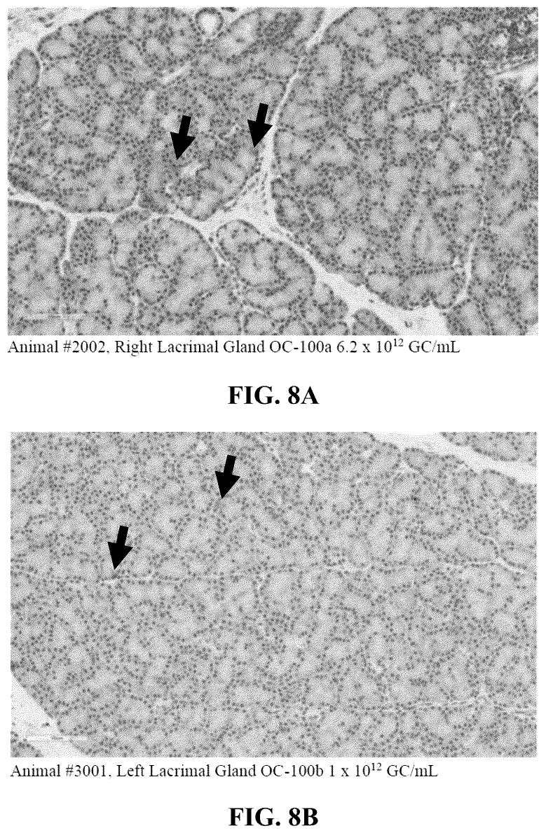

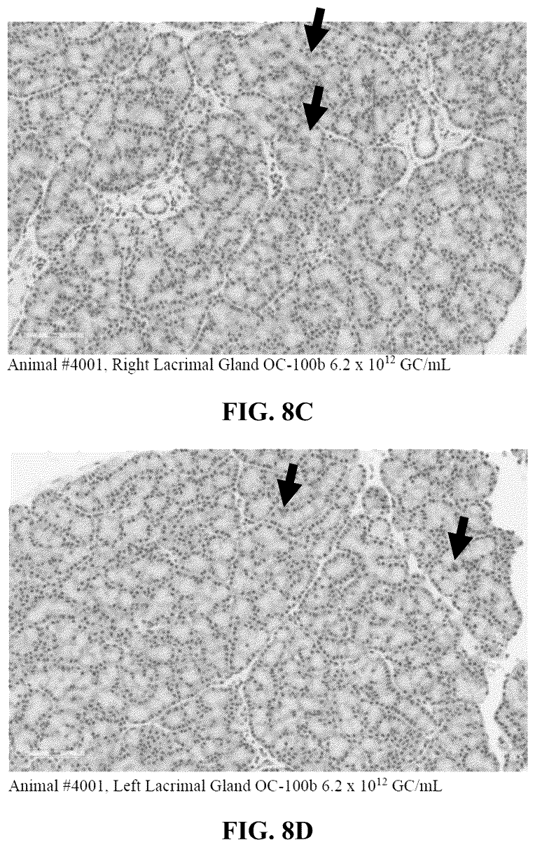

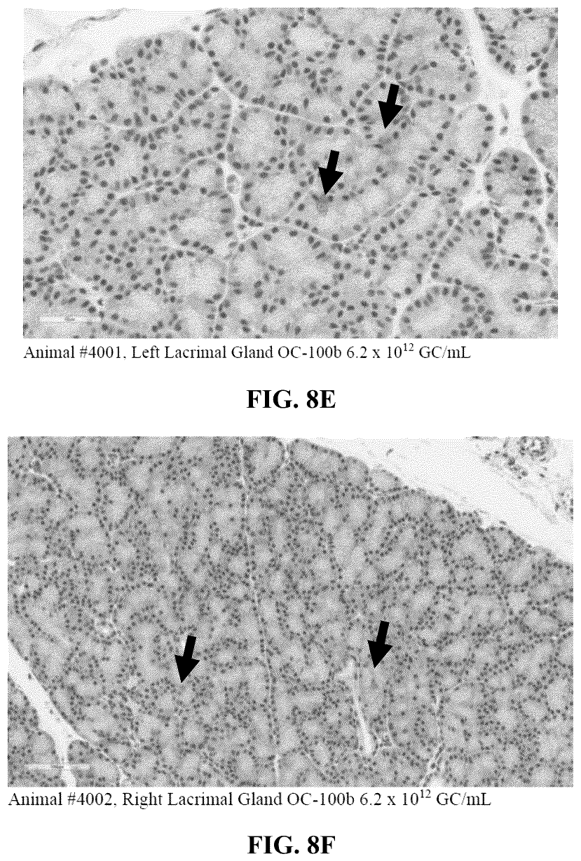

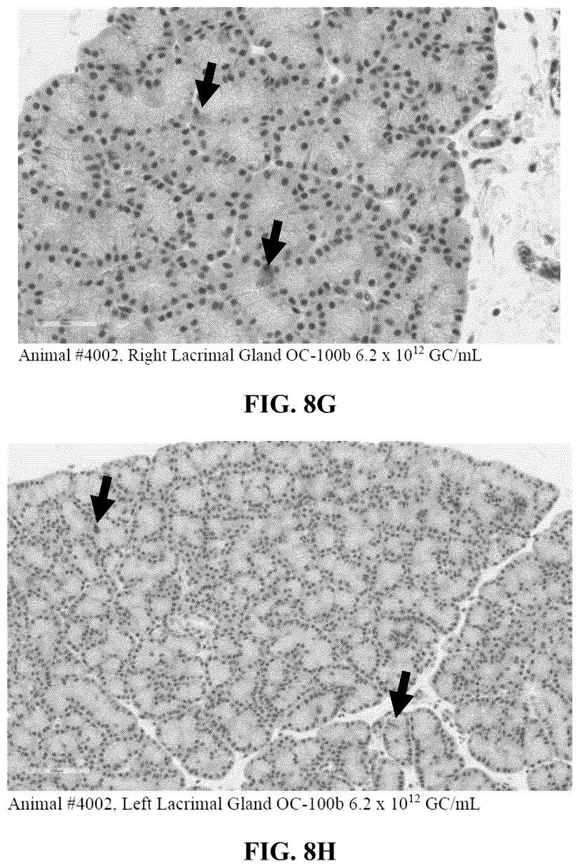

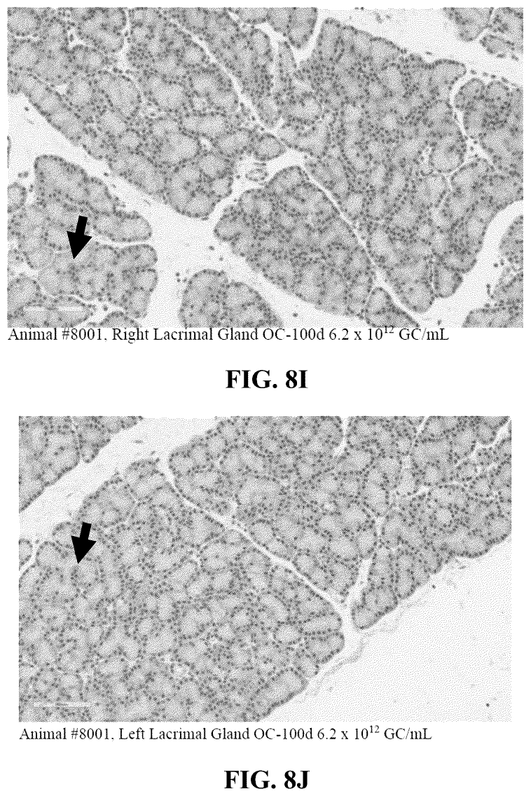

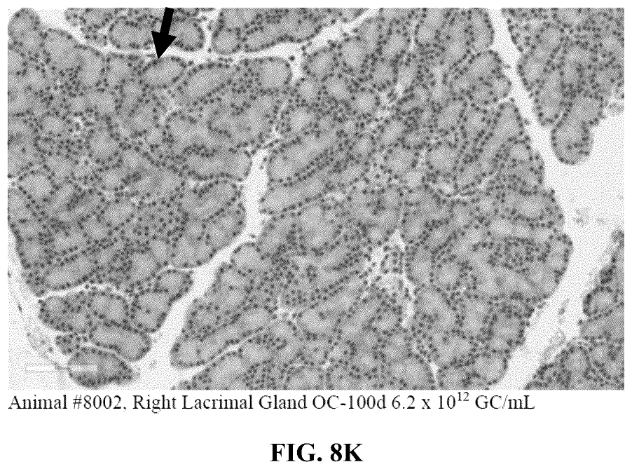

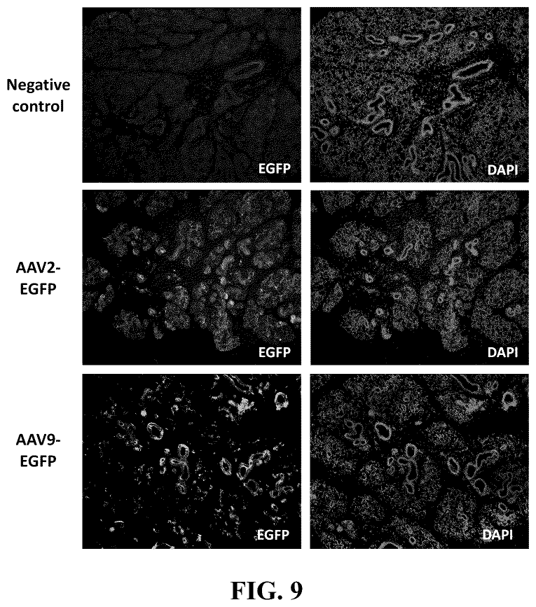

Microscopic evaluation was performed to determine the efficiency of eGFP expression in lacrimal gland tissues dosed in vivo with the rAAV compositions. Isolated positive acinar cells in the IHC samples had pink to red cytoplasmic staining indicative of GFP expression (FIGS. 8A-8K; exemplary staining indicated by black arrows). Positive eGFP expression was observed in for the rAAV composition containing an AAV2 capsid protein (OC-100a) at 6.2×10¹² GC/mL (FIG. 8A), the rAAV composition containing an AAV5 capsid protein at both 1×10¹² GC/mL (FIG. 8B) and 6.2×10¹² GC/mL (FIGS. 8C-8H), and the rAAV composition containing an AAV9 capsid protein at 6.2×10¹² GC/mL (FIGS. 8I-8K).

### Conclusion

The results from this example show that rAAV vectors can be used to deliver an expression cassette to the lacrimal gland by direct injection. The results also show that rAAV vectors containing capsid proteins with at least the AAV2, AAV5, or AAV9 serotypes can be used to deliver an expression cassette to cells within the lacrimal gland. Furthermore, the results demonstrate delivery of an expression cassette containing a transgene operatively linked to a CAG promoter sequence results in expression of the transgene in the cells of the lacrimal gland.

### Example 3: Expression of EGFP Transgene in Porcine Lacrimal Gland Delivered by rAAV via Intralacrimal Injection

The study objective was to assess if the lacrimal gland is able to be leveraged as a method to modify or enrich the tear film with a protein of interest in pigs. Subsequently, in vivo study was performed to test if EGFP could be produced in the acinar cells of the lacrimal gland and then secreted into the tear film after delivery of an adenoviral vector consisting of a plasmid encoding eGFP. To get cDNA encoding EGFP into acinar cells, the approach was to inject the lacrimal gland with an adeno-associated virus (AAV) which contained cDNA encoding for secreted EGFP (secEGFP). To create each AAV of 2 different serotypes (2 and 9) for secEGFP, an AAV transfer plasmid was generated which contained between the inverted terminal repeats (ITRs) the essential elements for secEGFP expression. The DNA sequence between the ITRs was packaged into the AAV (FIG. 7) that was manufactured.

### Design Analysis and Methodology

Research grade AAVs for secreted EGFP (serotypes 2 and 9) were synthesized at Sirion and were provided at a stock concentration of 5×10¹². The AAVs were in vitro tested by CJ Solutions using HEK 293T cells and ELISA to ensure that the manufactured AAVs would transduce cells. At Texas A&M, eight domestic pigs received a one-time intralacrimal gland injection of EGFP with the right (OD; oculus dexter) gland receiving a low dose and the left (OS; oculus sinister) gland receiving a high dose. Six weeks after the first injection, a second injection with AAV2 and AAV9 high doses were performed. The study assessed EGFP expression at Day 35. Following tear EGFP level confirmation, the study was terminated 8 weeks after the second injection to assess the presence of EGFP in the lacrimal glands and assess any potential inflammation or gland abnormalities. (Tables 8 & 9). In the study, nasal spray dosing was administered between weeks 3 to 4 (Table 10).

TABLE 8
Study plan for in vivo study of AAV2-secEGFP and AAV9-secEGFP in domestic pigs.

| Title | | Fifteen week porcine study of AAV2-secEGFP and AAV9- |
|---|---|---|
| | | secEGFP following repeat intralacrimal gland injection and |
| | | OC-01 nasal spray administration. |
| GLP Status | | No |
| Objective(s) | | Fifteen week in vivo study to assess lacrimal gland |
| | | transduction following two intralacrimal gland injections |
| | | given 6 weeks apart. An initial of low-dose (1 × 10⁹ vg)- or |
| | | high-dose (1 × 10¹¹ vg) of AAV2-secEGFP or AAV9-secEGFP |
| | | was administered at Day 0 to transduce the porcine lacrimal |
| | | gland followed by a high dose (1 × 10¹¹ vg) at week 6 (Day |
| | | 47). The results are needed to: 1) confirm expression and |
| | | secretion of EGFP; 2) determine if EGFP is detectable up to 8 |
| | | weeks post-injection; and 3) assess any AAV related safety |
| | | signals following repeat administration. |
| Study | Breed | Domestic pig |
| Animals | Species | Sus scrofa domesticus |
| | Age | 8-10 weeks |
| | Body Weight | ~18-23 kg |
| | Sex-(#M/#F) | 8 males |
| | Total | 8 for study (+naïve) |

| Test article(s) | First Injection- AAV2-secEGFP injected at a 1.0 × 10⁹ vg | |
|---|---|---|
| | dose (OD) or 1.0 × 10¹¹ vg dose (OS) into the lacrimal gland | |
| | AAV9-secEGFP injected at a 1.0 × 10⁹ vg dose (OD) or | |
| | 1.0 × 10¹¹ vg dose (OS) into the lacrimal gland | |
| | Second injection: AAV2-secEGFP and AAV9-secEGFP at a | |
| | 1 × 10¹¹ vg dose | |
| Route of Administration | Intralacrimal gland injection for AAV and intranasal dose | |
| | OC-01 | |
| Study Groups and Dose | See Table Below | |
| Levels | | |
| Dosing Regimen | 1. | First injection into each lacrimal gland; |
| | | OD: single injection of 40 μL low dose |
| | | OS: single injection of 40 μL high dose |
| | 2. | Nasal spray given twice daily from day 21 to day 28 |
| | 3. | Second injection into each lacrimal gland |

| | |
|---|---|
| | 4. |
| Randomization | No |
| Planned Sacrifice Timepoint | Day 103 |

TABLE 9
Injected dose and volume of AAV.

| | | | | | | Number of |
|---|---|---|---|---|---|---|
| | | | Dose number | | | Animals |
| | Dose Level | Injection | and volume | Route | Route | (gender ) |
| Group | (vg) | Time | (μL) | (Right Gland) | (Left Gland) | M |
| 1 | OD: low dose | Day 0 | OD: 40 μL | Intralacrimal | Intralacrimal | 4 |
| | 1.0 × 10⁹ | | single injection | AAV2-secEGFP | AAV2-secEGFP | |
| | OS: high dose | | OS: 40 μL | OD: 40 μL | OS: 40 μL | |
| | 1.0 × 10¹¹ | | single injection | single injection | single injection | |
| | | | | low dose | high dose | |
| 2 | OD: low dose | Day 0 | OD: 40 μL | Intralacrimal | Intralacrimal | 4 |
| | 1.0 × 10⁹ | | single injection | AAV9-secEGFP | AAV9-secEGFP | |
| | OS: high dose | | OS: 40 μL | OD: 40 μL | OS: 40 μL | |
| | 1.0 × 10¹¹ | | single injection | single injection | single injection | |
| | | | | low dose | high dose | |
| 1 | OD: high dose | Day 47 | OD: 40 μL | Intralacrimal | Intralacrimal | 4 |
| | 1 × 10¹¹ | | single injection | AAV9-secEGFP | AAV9-secEGFP | |
| | OS: high dose | | OS: 40 μL | OD: 40 μL | OS: 40 μL | |
| | 1.0 × 10¹¹ | | single injection | single injection | single injection | |
| 2 | OD: high dose | Day 47 | OD: 40 μL | Intralacrimal | Intralacrimal | 4 |
| | 1 × 10¹¹ | | single injection | AAV2-secEGFP | AAV2-secEGFP | |
| | OS: high dose | | OS: 40 μL | OD: 40 μL | OS: 40 μL | |
| | 1.0 × 10¹¹ | | single injection | single injection | single injection | |

vg = viral genomes

TABLE 10
OC-01 nasal spray dosing was from day 21 to day 28.

| | | | | Number |
|---|---|---|---|---|
| | | | | of |
| | | | | Animals |
| | OC-01 | | Dose | (gender) |
| Group | Dose Level | Dose | number | Male |
| 1- Bilateral | 0.05 mL | BID | Twice daily from | 4 |
| nares dose | of 30 mcg | | day 21-28 | |
| 2- Bilateral | 0.05 mL | BID | Twice daily | 4 |
| nares dose | of 30 mcg | | from day 21-28 | |

mcg = micrograms

Following the second AAV-secEGFP injection, tears were collected from each eye via Schirmer strips on day 82. Tears were collected by placing a Schirmer's Tear Test strip in the lower conjunctival cul-de-sac and leaving in place for 2 minutes. Tear protein was extracted from the Schirmer's Tear Test strip and mesoscale discovery (MSD) analysis was conducted to detect the presence of EGFP protein in the tears.

Lacrimal gland was collected for ocular histopathology on Day 103 and samples were sent to Zyagen, Inc. (San Diego, CA) for EGFP immunohistochemistry (IHC).

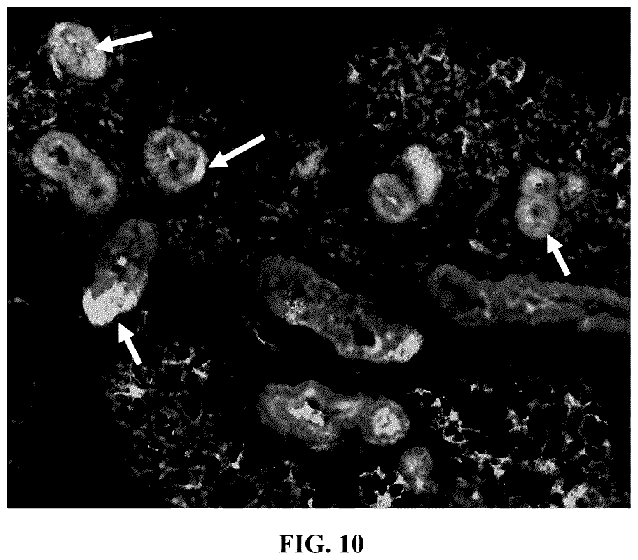

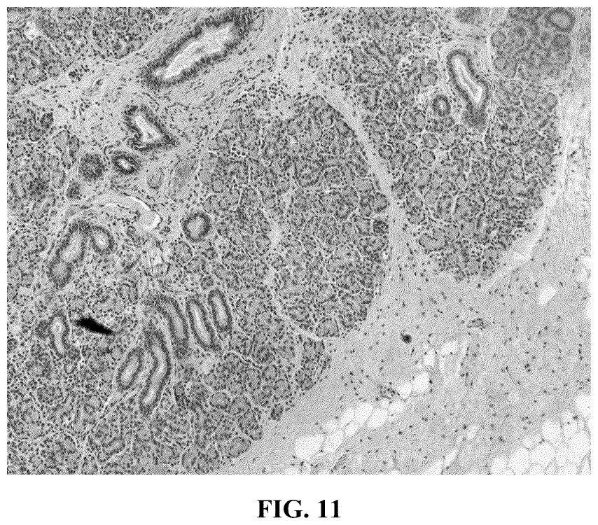

ELISA results showed that the AAV serotypes that were manufactured could transduce HEK 293T cells and produced secreted EGFP in vitro. EGFP expression in the tear samples was confirmed by MSD analysis 82 days after AAV transduction with eGFP some levels >400 pg/mL as well as by IHC (FIG. 9). IHC indicated that EGFP expression was within the acinar cells with greater acinar cell infectivity observed for AAV2 compared to AAV9. Additionally, transduction of ductile epithelial cells was observed for AAV9 injected lacrimal glands (FIG. 10). Hematoxylin and eosin staining of pig lacrimal gland after repeat AAV injections did not show any inflammatory infiltrate, atrophy or edema (FIG. 11).

Porcine lacrimal gland that is injected with either AAV2-secEGFP or AAV9-secEGFP expressed the EGFP transgene product in acinar cells as well as ductile epithelial cells. The EGFP that was expressed in the lacrimal gland was found to be secreted into the tear film. Additionally, no safety signals or inflammatory infiltrates were observed in any animals after repeat injections of AAV2 or AAV9 regardless if they initially received a low or high dose of AAV during the first injection. The results of this study demonstrate that the acinar cells of the lacrimal gland are a target for a gene therapy approach to modify and/or enrich the tear film.

### Example 4: Expression of rAAV Transgene in Porcine Lacrimal Gland in Combination with OC-1 Nasal Spray

This example describes a study in pigs to assess expression of rAAV encoding a model protein following a single intralacrimal gland injection. The objective of the study is to determine expression of mRNA encoding the model protein (referred to in the Examples and Figures as “protein_A”) in the lacrimal gland and transgene protein levels in tears following a one-time injection of an AAV encoding protein_A (referred to in the Examples and Figures as “AAV-protein_A”) to transduce the porcine lacrimal gland. The study is further designed to confirm expression and secretion of protein_A following intralacrimal gland injection and to assess the relative amount of protein present on the ocular surface upon stimulating tear production with varenicline nasal spray. Varenicline (“OC-1”) is the following compound:

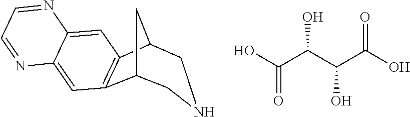 [SMILES: c1cnc2cc3c(cc2n1)C1CNCC3C1 ; O=C(O)[C@H](O)[C@@H](O)C(=O)O]

The AAV-protein_A plasmid encodes from 5′ to 3′ an AAV2 5′ITR, a CMV enhancer/promoter, an intronic sequence comprising a Kozak, an open reading frame encoding protein_A, a Woodchuck Hepatitis Virus Posttranscriptional Regulatory Element, polyA sequence, and an AAV2 3′ITR.

The study parameters are detailed in Table 11. The administration and dosing are shown in Table 12.

TABLE 11
Study plan for in vivo evaluation of intralacrimal
gland injection of rAAV in domestic pigs

| Title | | Porcine study of AAV-protein_A expression following a one- |
|---|---|---|
| | | time intralacrimal gland injection. |
| Objective(s) | | In vivo study to determine protein_A mRNA lacrimal gland |
| | | expression, and protein_A tear protein levels following a |
| | | one-time injection of an AAV-protein_A to transduce the |
| | | porcine lacrimal gland. The results will confirm expression |
| | | and secretion of protein A. |
| Study | Breed | Domestic pig |
| Animals | Species | Sus scrofa domesticus |
| | Body Weight | ~40 pounds on Day 1 |
| | Sex-(#M/#F) | 10 study animals (5M/5F) incl. 2 non-injected control animals |
| | Total | 10 |

| Test article(s) | AAV-protein_A injected at 1.0 × 10¹¹ vg dose into both | |
|---|---|---|
| | lacrimal glands | |
| | OC-01 (varenicline) nasal spray administered as a 0.05mL | |
| | spray containing 30 mcg varenicline | |
| Route of Administration | Intralacrimal gland injection (AAV-protein_A) and | |
| | intranasal (OC-01) | |
| Study Groups and Dose | See Table Below | |
| Levels | | |
| Dosing Regimen | 1. | One-time injection into each lacrimal gland; |
| | | OD: single injection of 40 μL |
| | | OS: single injection of 40 μL |
| | 2. | Nasal spray given twice daily from day 22 to day 27 |
| | | (at least 6 hours apart) and once on day 28. |
| | 3. | Nasal spray will be administered once on days 14, 28, |
| | | and 42 and 2 minutes after nasal spray administration |
| | | the tear collection will occur via Schirmer's Tear Test |
| | | Strips |

| | |
|---|---|
| Randomization | No |
| Planned Sacrifice Timepoint | Day 90 |

TABLE 12
Injected dose and volume of AAV

| | | | | Lacrimal | # | |
|---|---|---|---|---|---|---|
| Group | | Test | Dose level | gland | Injections | # of |
| # | Strain | Article | (vg/animal) | (LG) | per LG | animals/pigs |
| 1 | Sus scrofa | AAV- | OD: 1 × 10¹¹ | Right | 1 per LG | 4 (8 eyes) |
| | domesticus | Protein_A | OS: 1 × 10¹¹ | Left | (40 μL) | |
| 2 | Sus scrofa | OC-01 | 30 mcg | N/A | 0 | 4 (8 eyes) |
| | domesticus | | intranasal | | | |
| 3 | Sus scrofa | Control | N/A | N/A | 0 | 2 (4 eyes) |
| | domesticus | | | | | |

AAV-protein A stock concentrations are 5 × 10¹² vg/mL and are provided in 500 μL aliquots and stored at −20° C. (short-term, <2 years) or −80° C. (long-term). In this study, there is only 1 dose, (1 × 10¹¹ vg).

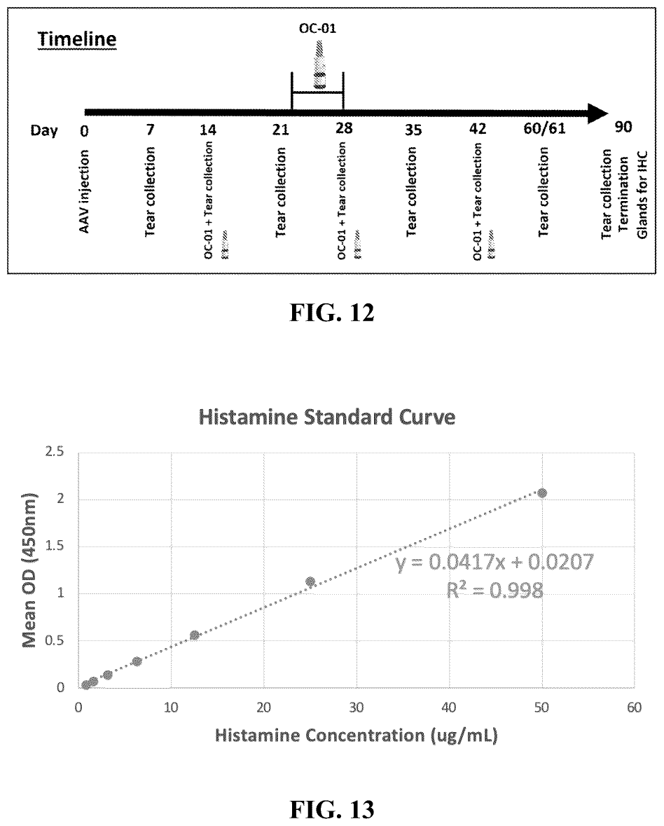

The timeline for the porcine study is shown in FIG. 12. For all pigs (N=14), day 0 is the day of injection. Tears are collected from each eye via Schirmer strips (applied for approximately 2 minutes then removed) on days 7, 14, 21, 28, 35, 42, 60/61, and 90. Schirmer's Strips are immediately cut above the fluid or dye line with a pair of scissors. The bottom portion (tear saturated) of the Schirmer's Strip is then placed in a microcentrifuge tube and kept on ice until transferred to freezer (−80° C.). On days 14 and 42, tear collection occurs first and then nasal spray dose is administered. Approximately 2 minutes after administration of nasal spray dose a second day tear collection occurs. For days 22 to 28, OC-01 nasal spray is administered twice daily (at least 6 hours between administrations) to both nares on days 22 to 27 and once on day 28. Tear collection on day 28 will occur 2 minutes after administration of nasal spray.

On day 90, gross pathology is performed and body weights are assessed. Additionally, one lacrimal gland from each animal is collected for ocular histopathology by immunohistochemistry (IHC). After harvesting the lacrimal glands, they are fixed in 10% formalin for 24 to 48 hours (at room temperature) then to 70% EtOH and store at 4° C. The volume in the tubes is maintained at 5 times that of the tissue and the tissue is fully submerged.

A second lacrimal gland is collected, rinsed with phosphate-buffered saline and immediately placed in RNA-Later. The sample is snap frozen in liquid nitrogen. ˜0.5-1 cm square pieces of tissue from heart apex, kidney and liver are collected and immediately placed in a 2 mL cryotube and snap frozen in liquid nitrogen. Cryotubes are later stored at −80° C. until shipped for mRNA analysis.

mRNA analysis is performed to analyze AAV-derived gene expression of protein_A in the lacrimal glands of domestic pigs, using a developed one-step duplex RT-qPCR method. Each lacrimal gland is homogenized and lysates loaded into a QIASymphony for automated RNA extraction utilizing silica-based RNA purification, magnetic separation and enzymatic removal of DNA. The AAV-protein_A vectors contain the bovine growth hormone (bGH) polyA sequence at the 3′ untranslated region of the transgene. Protein_A mRNA is analyzed using primers and a probe targeting the bGH poly A sequence, with sequences shown in Table 13. Amplification is for 76 bp of the bGH sequence. The extracted total RNA samples are analyzed for both bGH mRNA copy numbers and the Ct values of porcine endogenous Hprt1 mRNA on 96-well plates using the QuantStudio 7 Flex Real Time PCR system and a one-step duplex RT-qPCR method (see Table 14). Each plate includes a standard curve, negative controls and quality control samples, which are prepared separately to avoid cross-contamination. Each standard curve includes bGH standard DNA levels at 108, 107, 105, 104, 103, 102, 50, 25 and 0 copies per well. RT-qPCR of the RNA samples is performed in duplicate wells up to 100 ng per well. The bGH mRNA copy number in each RT-qPCR well is interpolated from the bGH DNA standard curve (Acceptance Criteria: R2≥0.980). A two-fold multiplication step is used to adjust for interpolating single stranded (ss) mRNA from the double stranded (ds) standard curve, and the mean copy number of the two replicate wells will be reported as copies of ss bGH mRNA per 100 ng of RNA sample. In addition, each RNA sample is tested using qScript XLT One-step RT-qPCR (without added reverse transcriptase) to monitor the potential vector DNA contamination in the RNA samples.

TABLE 13
primer and probe sequences for bGH sequence

| | | SEQ |
|---|---|---|
| | | ID |
| Name | Sequence | NO |
| bGH-F3 Forward | 5′-ACTCCCACTGTCC | 32 |
| Primer | TTTCCTAATAAAA-3′ | |
| bGH-R3 Reverse | 5′-CCCCCAGAATAGA | 33 |
| Primer | ATGACACCTA-3′ | |
| bGH-P3 Probe | 5′-FAM-TTGCATCGC | 34 |
| italics = fluorescent | ATTGTC-MGBNFQ-3′ | |
| dye | | |
| bold = quencher | | |

TABLE 14
RT-qPCR assay conditions

| | Final |
|---|---|
| Reagent | concentration |
| 2x qScript XLT One-Step | 1x |
| ToughMix Master Mix* | |

| | | |
|---|---|---|
| bGH-F3 Forward Primer | 200 | nM |
| bGH-R3 Reverse Primer | 800 | nM |
| bGH-P3 Probe | 150 | nM |
| Porcine Hprt1-F1 Primer | 200 | nM |
| Porcine Hprt1-R1 Primer | 800 | nM |
| Porcine Hprt1-P1 Probe | 150 | nM |
| Porcine lacrimal gland matrix RNA** | 100 | ng |
| bGH Standard DNA** | 0-10⁸ | copies |

| | |
|---|---|
| Nuclease-free water | To final volume of 20 μL |

*ToughMix is replaced with One-Step RT-qPCR Master Mix, without reverse transcriptase, when analyzing RNA samples for monitoring potential vector DNA contamination.

### Example 5: Expression of Functional DAO1 in 293T Cells

This example describes a study which evaluated the expression of a DAO1 transgene in 293T cells using a colorimetric histamine quantification assay. The objective of the study was to confirm whether transfection resulted in the expression of functional DAO1.

### Background

There are five established biogenic amine neurotransmitters identified to date, which include dopamine, norepinephrine, epinephrine, histamine and serotonin formed by decarboxylation of amino acids or an amination event of ketones or aldehydes. Histamine, in particular, is formed by the decarboxylation of histidine and described over 115 years ago (Windaus A, Vogt W. “Synthese des Imidazolyl-athylamins.” Ber. Dtsch. Chem. Ges. 1907; 40:3691-3695). The degradation of histamine by DAO1 primarily occurs extracellularly, which is the most relevant from a therapeutic approach when trying to modulate a histamine response from histamine ingestion or localized mast cell degranulation resulting in histamine release into the extracellular matrix and systemic circulation. One mechanism by which histamine is degraded by DAO1 is through oxidative deamination of histamine. Oxidation is the first step in degrading/metabolizing histamine for eventual release of the byproducts from the body in the form of urine components.

### DAO1 Enzymatic Activity Assay

In this assay, 293T cells transfected with a plasmid engineered to express DAO1, and non-transfected 293T cells (a negative control) were generated and/or cultured using standard protocols, and conditioned media from each sample was collected and concentrated using Amicon® protein concentrators. DAO1 enzymatic activity was evaluated using a Sigma-Aldrich® Histamine MAK432 Histamine Quantification Assay Kit, subject to a modified protocol, as explained below.

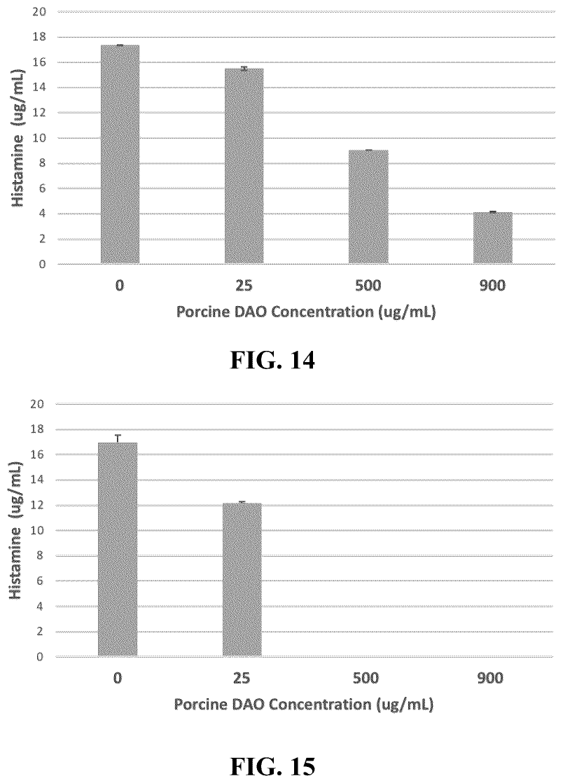

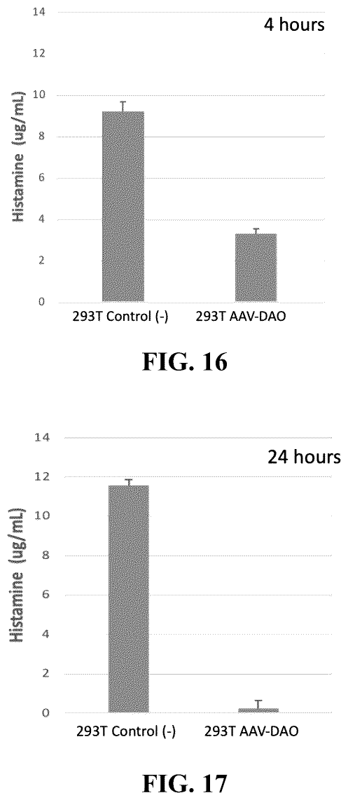

The MAK432 kit provides a colorimetric method for the detection of total histamine from various sources. This kit is based on the enzymatic oxidation of histamine, which is coupled to the reduction of the included formazan WST reagent. The intensity of the product color, measured at 450 nm, is directly proportional to the histamine concentration in the sample. The MAK432 kit includes a “Reaction Enzyme” component to oxidize the test sample and histamine standards that can be used to prepare a histamine standard curve. For the purposes of determining if the DAO1 transgene was functional and able to oxidize histamine, the manufacturer's protocol was modified by eliminating the use of the provided Reaction Enzyme. Briefly, 25 μg/ml histamine and phosphate buffered saline were combined with 600 μL of conditioned media (from the 293T cells transfected with the DAO1 plasmid, or the negative control 293T cells) or recombinant porcine DAO1 (used as a positive control) in a 5 mL reaction volume. The reaction time of 4 or 24 hours was conducted at 37° C. on a 150 rpm rotary shaker. The manufacturer's detection solution was then added and stopped after 35 minutes. FIG. 13 1 shows the standard curve generated for histamine with an R² value of 0.998. FIGS. 14-15 demonstrate the kinetics of histamine degradation by increasing concentrations of recombinant porcine DAO1 at 4 (FIG. 14) and 24 (FIG. 15) hours. FIGS. 16-17 demonstrate the ability of the 293T cell conditioned media transfected with the DAO1 plasmid to significantly degrade histamine after 4 (FIG. 16) 24 (FIG. 17) hours of exposure, as compared to the non-transfected negative control.

As illustrated by the provided histamine level data, transfection with a DAO1 expression plasmid resulted in a substantial reduction in histamine levels as compared to the negative control (e.g., a reduction of >50% after 4 hours, and by >90% after 24 hours), confirming that transfection of 293T cells with the DAO1 plasmid resulted in the expression of functional DAO1.

While embodiments of the present invention have been shown and described herein, those skilled in the art will understand that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

SEQUENCE LISTING

| | | SEQ |
|---|---|---|
| Name/ | | ID |
| Identifier | Sequence | NO |
| Human DAO1 | MPALGWAVAAILMLQTAMAEPSPGTLPRKAGVFSDLSNQELKAVH | 1 |
| amino acid | SFLWSKKELRLQPSSTTTMAKNTVFLIEMLLPKKYHVLRFLDKGE | |
| sequence 1 | RHPVREARAVIFFGDQEHPNVTEFAVGPLPGPCYMRALSPRPGYQ | |
| | SSWASRPISTAEYALLYHTLQEATKPLHQFFLNTTGFSFQDCHDR | |
| | CLAFTDVAPRGVASGQRRSWLIIQRYVEGYFLHPTGLELLVDHGS | |
| | TDAGHWAVEQVWYNGKFYGSPEELARKYADGEVDVVVLEDPLPGG | |
| | KGHDSTEEPPLFSSHKPRGDFPSPIHVSGPRLVQPHGPRFRLEGN | |
| | AVLYGGWSFAFRLRSSSGLQVLNVHFGGERIAYEVSVQEAVALYG | |
| | GHTPAGMQTKYLDVGWGLGSVTHELAPGIDCPETATFLDTFHYYD | |
| | ADDPVHYPRALCLFEMPTGVPLRRHFNSNFKGGFNFYAGLKGQVL | |
| | VLRTTSTVYNYDYIWDFIFYPNGVMEAKMHATGYVHATFYTPEGL | |
| | RHGTRLHTHLIGNIHTHLVHYRVDLDVAGTKNSFQTLQMKLENIT | |
| | NPWSPRHRVVQPTLEQTQYSWERQAAFRFKRKLPKYLLFTSPQEN | |
| | PWGHKRTYRLQIHSMADQVLPPGWQEEQAITWARTEGGQPRALSQ | |
| | AASPVPGRYPLAVTKYRESELCSSSIYHQNDPWHPPVVFEQFLHN | |
| | NENIENEDLVAWVTVGFLHIPHSEDIPNTATPGNSVGFLLRPFNF | |
| | FPEDPSLASRDTVIVWPRDNGPNYVQRWIPEDRDCSMPPPFSYNG | |
| | TYRPV | |
| Human DAO1 | MPALGWAVAAILMLQTAMAEPSPGTLPRKAGVFSDLSNQELKAVH | 2 |
| amino acid | SFLWSKKELRLQPSSTTTMAKNTVFLIEMLLPKKYHVLRFLDKGE | |
| sequence 2 | RHPVREARAVIFFGDQEHPNVTEFAVGPLPGPCYMRALSPRPGYQ | |
| | SSWASRPISTAEYALLYHTLQEATKPLHQFFLNTTGFSFQDCHDR | |
| | CLAFTDVAPRGVASGQRRSWLIIQRYVEGYFLHPTGLELLVDHGS | |
| | TDAGHWAVEQVWYNGKFYGSPEELARKYADGEVDVVVLEDPLPGG | |
| | KGHDSTEEPPLFSSHKPRGDFPSPIHVSGPRLVQPHGPRFRLEGN | |
| | AVLYGGWSFAFRLRSSSGLQVLNVHFGGERIAYEVSVQEAVALYG | |
| | GHTPAGMQTKYLDVGWGLGSVTHELAPGIDCPETATFLDTFHYYD | |
| | ADDPVHYPRALCLFEMPTGVPLRRHFNSNFKGGFNFYAGLKGQVL | |
| | VLRTTSTVYNYDYIWDFIFYPNGVMEAKMHATGYVHATFYTPEGL | |
| | RHGTRLHTHLIGNIHTHLVHYRVDLDVAGTKNSFQTLQMKLENIT | |
| | NPWSPRHRVVQPTLEQTQYSWERQAAFRFKRKLPKYLLFTSPQEN | |
| | PWGHKRTYRLQIHSMADQVLPPGWQEEQAITWARYPLAVTKYRES | |
| | ELCSSSIYHQNDPWHPPVVFEQFLHNNENIENEDLVAWVTVGFLH | |
| | IPHSEDIPNTATPGNSVGFLLRPFNFFPEDPSLASRDTVIVWPRD | |
| | NGPNYVQRWIPEDRDCSMPPPFSYNGTYRPV | |
| Human DAO1 | GCAAAGGCTGGAAGCAGAGCGAACTGGGAGCAGAGCACACAGAGC | 3 |
| mRNA 2 | CGTGGAGCGAGAGATGCCGGCCCTGGGCTGGGCCGTGGCTGCCAT | |
| untranslated | CCTGATGCTGCAGACGGCCATGGCGGAGCCCTCCCCGGGGACTCT | |
| regions in | GCCCAGGAAGGCAGGGGTGTTTTCAGACCTAAGCAACCAAGAGCT | |
| bold | GAAGGCAGTGCACAGCTTCCTCTGGTCCAAGAAGGAGCTGAGGCT | |
| ORF in italics | GCAGCCCTCCAGTACCACCACCATGGCCAAGAACACCGTGTTTCT | |
| | CATCGAGATGCTGCTGCCCAAGAAGTACCATGTGCTGAGGTTTCT | |
| | GGATAAAGGTGAAAGGCATCCTGTGCGGGAAGCCCGTGCCGTCAT | |
| | CTTCTTTGGTGACCAGGAGCATCCCAATGTCACCGAGTTTGCTGT | |
| | GGGGCCCCTGCCAGGGCCCTGCTACATGCGAGCACTGTCCCCCAG | |
| | GCCTGGGTACCAGTCCTCCTGGGCATCGAGGCCCATCTCCACAGC | |
| | AGAGTATGCCCTCCTCTACCACACCCTGCAGGAAGCCACCAAGCC | |
| | CCTGCATCAGTTCTTCCTCAATACCACAGGCTTCTCATTCCAAGA | |
| | CTGCCATGACAGATGCCTGGCCTTCACCGATGTGGCCCCCCGGGG | |
| | TGTGGCTTCTGGCCAGCGCCGCAGTTGGCTTATCATACAGCGCTA | |
| | TGTAGAAGGCTACTTTCTGCACCCCACTGGGCTGGAGCTCCTCGT | |
| | GGATCATGGGAGCACAGATGCTGGGCACTGGGCCGTGGAGCAGGT | |
| | GTGGTACAACGGGAAGTTCTATGGGAGCCCAGAGGAACTGGCTCG | |
| | GAAGTATGCAGATGGAGAGGTGGACGTGGTGGTCCTGGAGGACCC | |
| | GCTGCCTGGGGGCAAGGGGCATGACAGCACAGAGGAGCCGCCCCT | |
| | CTTCTCCTCCCACAAGCCCCGCGGGGACTTCCCCAGCCCCATCCA | |
| | TGTGAGCGGCCCCCGCTTGGTCCAGCCCCACGGCCCTCGCTTCAG | |
| | GCTGGAGGGCAACGCTGTGCTCTACGGCGGCTGGAGCTTTGCCTT | |
| | CCGGCTGCGCTCCTCCTCCGGGCTGCAGGTCCTGAACGTGCACTT | |
| | CGGCGGAGAGCGCATTGCCTATGAGGTCAGCGTGCAAGAGGCAGT | |
| | GGCGCTGTATGGAGGACACACACCTGCAGGCATGCAGACCAAGTA | |
| | CCTCGATGTCGGCTGGGGCCTGGGCAGCGTCACTCATGAGTTAGC | |
| | CCCCGGCATCGACTGCCCGGAGACCGCCACCTTCCTGGACACTTT | |
| | CCACTACTATGATGCCGATGACCCGGTCCATTATCCCCGAGCCCT | |
| | CTGCCTCTTTGAAATGCCCACAGGGGTGCCCCTTCGGCGGCACTT | |
| | TAATTCCAACTTTAAAGGTGGCTTCAACTTCTATGCGGGGCTGAA | |
| | GGGCCAGGTGCTGGTGCTGCGGACAACTTCAACTGTCTACAATTA | |
| | TGATTACATTTGGGACTTTATCTTCTACCCCAACGGGGTGATGGA | |
| | GGCCAAGATGCATGCCACTGGCTACGTCCACGCCACCTTCTACAC | |
| | CCCCGAGGGGCTGCGCCACGGCACTCGCCTGCACACCCACCTGAT | |
| | TGGCAACATACACACTCACTTGGTGCACTACCGCGTAGACCTGGA | |
| | TGTGGCAGGCACCAAGAACAGCTTCCAGACACTGCAGATGAAGCT | |
| | AGAAAACATCACCAACCCCTGGAGCCCAAGACACCGCGTGGTCCA | |
| | GCCAACTCTGGAGCAGACGCAGTACTCCTGGGAGCGCCAGGCGGC | |
| | CTTCCGCTTCAAAAGGAAGCTGCCTAAGTACCTGCTCTTTACCAG | |
| | CCCCCAGGAGAACCCCTGGGGCCACAAGCGCACGTACCGCCTGCA | |
| | GATCCACTCCATGGCCGACCAGGTGCTGCCCCCAGGCTGGCAGGA | |
| | GGAGCAGGCCATCACCTGGGCAAGGACTGAGGGGGGCCAGCCCAG | |
| | GGCCCTGAGCCAAGCTGCTTCGCCTGTGCCTGGCAGGTACCCCCT | |
| | GGCAGTGACCAAGTACCGGGAGTCGGAGCTGTGCAGCAGCAGCAT | |
| | CTACCACCAGAACGACCCCTGGCACCCGCCCGTGGTCTTTGAGCA | |
| | GTTTCTTCACAACAACGAGAACATTGAAAATGAGGACCTGGTGGC | |
| | CTGGGTGACGGTGGGCTTCCTGCACATCCCCCACTCAGAGGACAT | |
| | TCCCAACACAGCCACACCTGGGAACTCCGTGGGCTTCCTGCTCCG | |
| | GCCATTCAACTTCTTCCCAGAGGACCCCTCCCTGGCATCCAGAGA | |
| | CACTGTGATCGTGTGGCCTCGGGACAACGGCCCCAACTACGTCCA | |
| | GCGCTGGATCCCTGAGGACAGGGACTGCTCGATGCCTCCCCCTTT | |
| | TAGCTACAATGGGACCTATAGACCTGTGTGACCAGCCCCCAGTTC | |
| | CTCCCCCAGTTCCTCCCAGGAAGCCCAGGAGCCTCACTGGGGCAG | |
| | ACAATAAACCCTCAGAGCCTCGCTCTGTGTGCTGCTTCTTGCGGG | |
| | GAGGCACAGGGCCATGTGTGTAGGAAACACACGAACAGACGTGCA | |
| | CACACACAGACGTGCACACACACACAGACATGCACACACACACAG | |
| | ACGTGCACACACACAGACGTGCACGCACTCACACGGACATGCACA | |
| | CACATGGCATGTACTTTATTCACACTGGTCTACTGCAGTCCAGAA | |
| | AAGCCACCATT | |
| Human DAO1 | GCAAAGGCTGGAAGCAGAGCGAACTGGGAGCAGAGCACACAGAGC | 4 |
| mRNA 2 | CGTGGAGCGAGAGATGCCGGCCCTGGGCTGGGCCGTGGCTGCCAT | |
| untranslated | CCTGATGCTGCAGACGGCCATGGCGGAGCCCTCCCCGGGGACTCT | |
| regions in | GCCCAGGAAGGCAGGGGTGTTTTCAGACCTAAGCAACCAAGAGCT | |
| bold | GAAGGCAGTGCACAGCTTCCTCTGGTCCAAGAAGGAGCTGAGGCT | |
| ORF in italics | GCAGCCCTCCAGTACCACCACCATGGCCAAGAACACCGTGTTTCT | |
| | CATCGAGATGCTGCTGCCCAAGAAGTACCATGTGCTGAGGTTTCT | |
| | GGATAAAGGTGAAAGGCATCCTGTGCGGGAAGCCCGTGCCGTCAT | |
| | CTTCTTTGGTGACCAGGAGCATCCCAATGTCACCGAGTTTGCTGT | |
| | GGGGCCCCTGCCAGGGCCCTGCTACATGCGAGCACTGTCCCCCAG | |
| | GCCTGGGTACCAGTCCTCCTGGGCATCGAGGCCCATCTCCACAGC | |
| | AGAGTATGCCCTCCTCTACCACACCCTGCAGGAAGCCACCAAGCC | |
| | CCTGCATCAGTTCTTCCTCAATACCACAGGCTTCTCATTCCAAGA | |
| | CTGCCATGACAGATGCCTGGCCTTCACCGATGTGGCCCCCCGGGG | |
| | TGTGGCTTCTGGCCAGCGCCGCAGTTGGCTTATCATACAGCGCTA | |
| | TGTAGAAGGCTACTTTCTGCACCCCACTGGGCTGGAGCTCCTCGT | |
| | GGATCATGGGAGCACAGATGCTGGGCACTGGGCCGTGGAGCAGGT | |
| | GTGGTACAACGGGAAGTTCTATGGGAGCCCAGAGGAACTGGCTCG | |
| | GAAGTATGCAGATGGAGAGGTGGACGTGGTGGTCCTGGAGGACCC | |
| | GCTGCCTGGGGGCAAGGGGCATGACAGCACAGAGGAGCCGCCCCT | |
| | CTTCTCCTCCCACAAGCCCCGCGGGGACTTCCCCAGCCCCATCCA | |
| | TGTGAGCGGCCCCCGCTTGGTCCAGCCCCACGGCCCTCGCTTCAG | |
| | GCTGGAGGGCAACGCTGTGCTCTACGGCGGCTGGAGCTTTGCCTT | |
| | CCGGCTGCGCTCCTCCTCCGGGCTGCAGGTCCTGAACGTGCACTT | |
| | CGGCGGAGAGCGCATTGCCTATGAGGTCAGCGTGCAAGAGGCAGT | |
| | GGCGCTGTATGGAGGACACACACCTGCAGGCATGCAGACCAAGTA | |
| | CCTCGATGTCGGCTGGGGCCTGGGCAGCGTCACTCATGAGTTAGC | |
| | CCCCGGCATCGACTGCCCGGAGACCGCCACCTTCCTGGACACTTT | |
| | CCACTACTATGATGCCGATGACCCGGTCCATTATCCCCGAGCCCT | |
| | CTGCCTCTTTGAAATGCCCACAGGGGTGCCCCTTCGGCGGCACTT | |
| | TAATTCCAACTTTAAAGGTGGCTTCAACTTCTATGCGGGGCTGAA | |
| | GGGCCAGGTGCTGGTGCTGCGGACAACTTCAACTGTCTACAATTA | |
| | TGATTACATTTGGGACTTTATCTTCTACCCCAACGGGGTGATGGA | |
| | GGCCAAGATGCATGCCACTGGCTACGTCCACGCCACCTTCTACAC | |
| | CCCCGAGGGGCTGCGCCACGGCACTCGCCTGCACACCCACCTGAT | |
| | TGGCAACATACACACTCACTTGGTGCACTACCGCGTAGACCTGGA | |
| | TGTGGCAGGCACCAAGAACAGCTTCCAGACACTGCAGATGAAGCT | |
| | AGAAAACATCACCAACCCCTGGAGCCCAAGACACCGCGTGGTCCA | |
| | GCCAACTCTGGAGCAGACGCAGTACTCCTGGGAGCGCCAGGCGGC | |
| | CTTCCGCTTCAAAAGGAAGCTGCCTAAGTACCTGCTCTTTACCAG | |
| | CCCCCAGGAGAACCCCTGGGGCCACAAGCGCACGTACCGCCTGCA | |
| | GATCCACTCCATGGCCGACCAGGTGCTGCCCCCAGGCTGGCAGGA | |
| | GGAGCAGGCCATCACCTGGGCAAGGTACCCCCTGGCAGTGACCAA | |
| | GTACCGGGAGTCGGAGCTGTGCAGCAGCAGCATCTACCACCAGAA | |
| | CGACCCCTGGCACCCGCCCGTGGTCTTTGAGCAGTTTCTTCACAA | |
| | CAACGAGAACATTGAAAATGAGGACCTGGTGGCCTGGGTGACGGT | |
| | GGGCTTCCTGCACATCCCCCACTCAGAGGACATTCCCAACACAGC | |
| | CACACCTGGGAACTCCGTGGGCTTCCTGCTCCGGCCATTCAACTT | |
| | CTTCCCAGAGGACCCCTCCCTGGCATCCAGAGACACTGTGATCGT | |
| | GTGGCCTCGGGACAACGGCCCCAACTACGTCCAGCGCTGGATCCC | |
| | TGAGGACAGGGACTGCTCGATGCCTCCCCCTTTTAGCTACAATGG | |
| | GACCTATAGACCTGTGTGACCAGCCCCCAGTTCCTCCCCCAGTTC | |
| | CTCCCAGGAAGCCCAGGAGCCTCACTGGGGCAGACAATAAACCCT | |
| | CAGAGCCTCGCTCTGTGTGCTGCTTCTTGCGGGGAGGCACAGGGC | |
| | CATGTGTGTAGGAAACACACGAACAGACGTGCACACACACAGACG | |
| | TGCACACACACACAGACATGCACACACACACAGACGTGCACACAC | |
| | ACAGACGTGCACGCACTCACACGGACATGCACACACATGGCATGT | |
| | ACTTTATTCACACTGGTCTACTGCAGTCCAGAAAAGCCACCATT | |
| CAG Promoter | ACTAGTTATTAATAGTAATCAATTACGGGGTCATTAGTTCATAGC | 5 |
| | CCATATATGGAGTTCCGCGTTACATAACTTACGGTAAATGGCCCG | |
| | CCTGGCTGACCGCCCAACGACCCCCGCCCATTGACGTCAATAATG | |
| | ACGTATGTTCCCATAGTAACGCCAATAGGGACTTTCCATTGACGT | |
| | CAATGGGTGGACTATTTACGGTAAACTGCCCACTTGGCAGTACAT | |
| | CAAGTGTATCATATGCCAAGTACGCCCCCTATTGACGTCAATGAC | |
| | GGTAAATGGCCCGCCTGGCATTATGCCCAGTACATGACCTTATGG | |
| | GACTTTCCTACTTGGCAGTACATCTACGTATTAGTCATCGCTATT | |
| | ACCATGGGTCGAGGTGAGCCCCACGTTCTGCTTCACTCTCCCCAT | |
| | CTCCCCCCCCTCCCCACCCCCAATTTTGTATTTATTTATTTTTTA | |
| | ATTATTTTGTGCAGCGATGGGGGCGGGGGGGGGGGGGGCGCGCGC | |
| | CAGGCGGGGCGGGGCGGGGCGAGGGGCGGGGCGGGGCGAGGCGGA | |
| | GAGGTGCGGCGGCAGCCAATCAGAGCGGCGCGCTCCGAAAGTTTC | |
| | CTTTTATGGCGAGGCGGCGGCGGCGGCGGCCCTATAAAAAGCGAA | |
| | GCGCGCGGCGGGCGGGAGTCGCTGCGTTGCCTTCGCCCCGTGCCC | |
| | CGCTCCGCGCCGCCTCGCGCCGCCCGCCCCGGCTCTGACTGACCG | |
| | CGTTACTCCCACAGGTGAGCGGGCGGGACGGCCCTTCTCCTCCGG | |
| | GCTGTAATTAGCGCTTGGTTTAATGACGGCTCGTTTCTTTTCTGT | |
| | GGCTGCGTGAAAGCCTTAAAGGGCTCCGGGAGGGCCCTTTGTGCG | |
| | GGGGGGAGCGGCTCGGGGGGTGCGTGCGTGTGTGTGTGCGTGGGG | |
| | AGCGCCGCGTGCGGCCCGCGCTGCCCGGCGGCTGTGAGCGCTGCG | |
| | GGCGCGGCGCGGGGCTTTGTGCGCTCCGCGTGTGCGCGAGGGGAG | |
| | CGCGGCCGGGGGCGGTGCCCCGCGGTGCGGGGGGGCTGCGAGGGG | |
| | AACAAAGGCTGCGTGCGGGGTGTGTGCGTGGGGGGGTGAGCAGGG | |
| | GGTGTGGGCGCGGCGGTCGGGCTGTAACCCCCCCCTGCACCCCCC | |
| | TCCCCGAGTTGCTGAGCACGGCCCGGCTTCGGGTGCGGGGCTCCG | |
| | TGCGGGGCGTGGCGCGGGGCTCGCCGTGCCGGGCGGGGGGTGGCG | |
| | GCAGGTGGGGGTGCCGGGCGGGGCGGGGCCGCCTCGGGCCGGGGA | |
| | GGGCTCGGGGGAGGGGCGCGGCGGCCCCGGAGCGCCGG | |
| AAV2 VP1 | CGGCTGTCGAGGCGCGGCGAGCCGCAGCCATTGCCTTTTATGGTA | 6 |
| protein | ATCGTGCGAGAGGGCGCAGGGACTTCCTTTGTCCCAAATCTGGCG | |
| | GAGCCGAAATCTGGGAGGCGCCGCCGCACCCCCTCTAGCGGGCGC | |
| | GGGCGAAGCGGTGCGGCGCCGGCAGGAAGGAAATGGGCGGGGAGG | |
| | GCCTTCGTGCGTCGCCGCGCCGCCGTCCCCTTCTCCATCTCCAGC | |
| | CTCGGGGCTGCCGCAGGGGGACGGCTGCCTTCGGGGGGGACGGGG | |
| | CAGGGCGGGGTTCGGCTTCTGGCGTGTGACCGGCGGCTCTAGAGC | |
| | CTCTGCTAACCATGTTCATGCCTTCTTCTTTTTCCTACAGCTCCT | |
| | GGGCAACGTGCTGGTTATTGTGCTGTCTCATCATTTTGGCAAAMA | |
| | ADGYLPDWLEDTLSEGIRQWWKLKPGPPPPKPAERHKDDSRGLVL | |
| | PGYKYLGPFNGLDKGEPVNEADAAALEHDKAYDRQLDSGDNPYLK | |
| | YNHADAEFQERLKEDTSFGGNLGRAVFQAKKRVLEPLGLVEEPVK | |
| | TAPGKKRPVEHSPVEPDSSSGTGKAGQQPARKRLNFGQTGDADSV | |
| | PDPQPLGQPPAAPSGLGTNTMATGSGAPMADNNEGADGVGNSSGN | |
| | WHCDSTWMGDRVITTSTRTWALPTYNNHLYKQISSQSGASNDNHY | |
| | FGYSTPWGYFDFNRFHCHFSPRDWQRLINNNWGFRPKRLNFKLFN | |
| | IQVKEVTQNDGTTTIANNLTSTVQVFTDSEYQLPYVLGSAHQGCL | |
| | PPFPADVFMVPQYGYLTLNNGSQAVGRSSFYCLEYFPSQMLRTGN | |
| | NFTFSYTFEDVPFHSSYAHSQSLDRLMNPLIDQYLYYLSRTNTPS | |
| | GTTTQSRLQFSQAGASDIRDQSRNWLPGPCYRQQRVSKTSADNNN | |
| | SEYSWTGATKYHLNGRDSLVNPGPAMASHKDDEEKFFPQSGVLIF | |
| | GKQGSEKTNVDIEKVMITDEEEIRTTNPVATEQYGSVSTNLQRGN | |
| | RQAATADVNTQGVLPGMVWQDRDVYLQGPIWAKIPHTDGHFHPSP | |
| | LMGGFGLKHPPPQILIKNTPVPANPSTTFSAAKFASFITQYSTGQ | |
| | VSVEIEWELQKENSKRWNPEIQYTSNYNKSVNVDFTVDTNGVYSE | |
| | PRPIGTRYLTRNL | |
| AAV2 VP1 | ATGGCTGCCGATGGTTATCTTCCAGATTGGCTCGAGGACACTCTC | 7 |
| ORF | TCTGAAGGAATAAGACAGTGGTGGAAGCTCAAACCTGGCCCACCA | |
| | CCACCAAAGCCCGCAGAGCGGCATAAGGACGACAGCAGGGGTCTT | |
| | GTGCTTCCTGGGTACAAGTACCTCGGACCCTTCAACGGACTCGAC | |
| | AAGGGAGAGCCGGTCAACGAGGCAGACGCCGCGGCCCTCGAGCAC | |
| | GACAAAGCCTACGACCGGCAGCTCGACAGCGGAGACAACCCGTAC | |
| | CTCAAGTACAACCACGCCGACGCGGAGTTTCAGGAGCGCCTTAAA | |
| | GAAGATACGTCTTTTGGGGGCAACCTCGGACGAGCAGTCTTCCAG | |
| | GCGAAAAAGAGGGTTCTTGAACCTCTGGGCCTGGTTGAGGAACCT | |
| | GTTAAGACGGCTCCGGGAAAAAAGAGGCCGGTAGAGCACTCTCCT | |
| | GTGGAGCCAGACTCCTCCTCGGGAACCGGAAAGGCGGGCCAGCAG | |
| | CCTGCAAGAAAAAGATTGAATTTTGGTCAGACTGGAGACGCAGAC | |
| | TCAGTACCTGACCCCCAGCCTCTCGGACAGCCACCAGCAGCCCCC | |
| | TCTGGTCTGGGAACTAATACGATGGCTACAGGCAGTGGCGCACCA | |
| | ATGGCAGACAATAACGAGGGCGCCGACGGAGTGGGTAATTCCTCG | |
| | GGAAATTGGCATTGCGATTCCACATGGATGGGCGACAGAGTCATC | |
| | ACCACCAGCACCCGAACCTGGGCCCTGCCCACCTACAACAACCAC | |
| | CTCTACAAACAAATTTCCAGCCAATCAGGAGCCTCGAACGACAAT | |
| | CACTACTTTGGCTACAGCACCCCTTGGGGGTATTTTGACTTCAAC | |
| | AGATTCCACTGCCACTTTTCACCACGTGACTGGCAAAGACTCATC | |
| | AACAACAACTGGGGATTCCGACCCAAGAGACTCAACTTCAAGCTC | |
| | TTTAACATTCAAGTCAAAGAGGTCACGCAGAATGACGGTACGACG | |
| | ACGATTGCCAATAACCTTACCAGCACGGTTCAGGTGTTTACTGAC | |
| | TCGGAGTACCAGCTCCCGTACGTCCTCGGCTCGGCGCATCAAGGA | |
| | TGCCTCCCGCCGTTCCCAGCAGACGTCTTCATGGTGCCACAGTAT | |
| | GGATACCTCACCCTGAACAACGGGAGTCAGGCAGTAGGACGCTCT | |
| | TCATTTTACTGCCTGGAGTACTTTCCTTCTCAGATGCTGCGTACC | |
| | GGAAACAACTTTACCTTCAGCTACACTTTTGAGGACGTTCCTTTC | |
| | CACAGCAGCTACGCTCACAGCCAGAGTCTGGACCGTCTCATGAAT | |
| | CCTCTCATCGACCAGTACCTGTATTACTTGAGCAGAACAAACACT | |
| | CCAAGTGGAACCACCACGCAGTCAAGGCTTCAGTTTTCTCAGGCC | |
| | GGAGCGAGTGACATTCGGGACCAGTCTAGGAACTGGCTTCCTGGA | |
| | CCCTGTTACCGCCAGCAGCGAGTATCAAAGACATCTGCGGATAAC | |
| | AACAACAGTGAATACTCGTGGACTGGAGCTACCAAGTACCACCTC | |
| | AATGGCAGAGACTCTCTGGTGAATCCGGGCCCGGCCATGGCAAGC | |
| | CACAAGGACGATGAAGAAAAGTTTTTTCCTCAGAGCGGGGTTCTC | |
| | ATCTTTGGGAAGCAAGGCTCAGAGAAAACAAATGTGGACATTGAA | |
| | AAGGTCATGATTACAGACGAAGAGGAAATCAGGACAACCAATCCC | |
| | GTGGCTACGGAGCAGTATGGTTCTGTATCTACCAACCTCCAGAGA | |
| | GGCAACAGACAAGCAGCTACCGCAGATGTCAACACACAAGGCGTT | |
| | CTTCCAGGCATGGTCTGGCAGGACAGAGATGTGTACCTTCAGGGG | |
| | CCCATCTGGGCAAAGATTCCACACACGGACGGACATTTTCACCCC | |
| | TCTCCCCTCATGGGTGGATTCGGACTTAAACACCCTCCTCCACAG | |
| | ATTCTCATCAAGAACACCCCGGTACCTGCGAATCCTTCGACCACC | |
| | TTCAGTGCGGCAAAGTTTGCTTCCTTCATCACACAGTACTCCACG | |
| | GGACAGGTCAGCGTGGAGATCGAGTGGGAGCTGCAGAAGGAAAAC | |
| | AGCAAACGCTGGAATCCCGAAATTCAGTACACTTCCAACTACAAC | |
| | AAGTCTGTTAATGTGGACTTTACTGTGGACACTAATGGCGTGTAT | |
| | TCAGAGCCTCGCCCCATTGGCACCAGATACCTGACTCGTAATCTG | |
| | TAA | |
| AAV2 VP3 | MATGSGAPMADNNEGADGVGNSSGNWHCDSTWMGDRVITTSTRTW | 8 |
| protein | ALPTYNNHLYKQISSQSGASNDNHYFGYSTPWGYFDFNRFHCHFS | |
| | PRDWQRLINNNWGFRPKRLNFKLFNIQVKEVTQNDGTTTIANNLT | |
| | STVQVFTDSEYQLPYVLGSAHQGCLPPFPADVFMVPQYGYLTLNN | |
| | GSQAVGRSSFYCLEYFPSQMLRTGNNFTFSYTFEDVPFHSSYAHS | |
| | QSLDRLMNPLIDQYLYYLSRTNTPSGTTTQSRLQFSQAGASDIRD | |
| | QSRNWLPGPCYRQQRVSKTSADNNNSEYSWTGATKYHLNGRDSLV | |
| | NPGPAMASHKDDEEKFFPQSGVLIFGKQGSEKTNVDIEKVMITDE | |
| | EEIRTTNPVATEQYGSVSTNLQRGNRQAATADVNTQGVLPGMVWQ | |
| | DRDVYLQGPIWAKIPHTDGHFHPSPLMGGFGLKHPPPQILIKNTP | |
| | VPANPSTTFSAAKFASFITQYSTGQVSVEIEWELQKENSKRWNPE | |
| | IQYTSNYNKSVNVDFTVDTNGVYSEPRPIGTRYLTRNL | |
| AAV2 VP3 | ATGGCTACAGGCAGTGGCGCACCAATGGCAGACAATAACGAGGGC | 9 |
| ORF | GCCGACGGAGTGGGTAATTCCTCGGGAAATTGGCATTGCGATTCC | |
| | ACATGGATGGGCGACAGAGTCATCACCACCAGCACCCGAACCTGG | |
| | GCCCTGCCCACCTACAACAACCACCTCTACAAACAAATTTCCAGC | |
| | CAATCAGGAGCCTCGAACGACAATCACTACTTTGGCTACAGCACC | |
| | CCTTGGGGGTATTTTGACTTCAACAGATTCCACTGCCACTTTTCA | |
| | CCACGTGACTGGCAAAGACTCATCAACAACAACTGGGGATTCCGA | |
| | CCCAAGAGACTCAACTTCAAGCTCTTTAACATTCAAGTCAAAGAG | |
| | GTCACGCAGAATGACGGTACGACGACGATTGCCAATAACCTTACC | |
| | AGCACGGTTCAGGTGTTTACTGACTCGGAGTACCAGCTCCCGTAC | |
| | GTCCTCGGCTCGGCGCATCAAGGATGCCTCCCGCCGTTCCCAGCA | |
| | GACGTCTTCATGGTGCCACAGTATGGATACCTCACCCTGAACAAC | |
| | GGGAGTCAGGCAGTAGGACGCTCTTCATTTTACTGCCTGGAGTAC | |
| | TTTCCTTCTCAGATGCTGCGTACCGGAAACAACTTTACCTTCAGC | |
| | TACACTTTTGAGGACGTTCCTTTCCACAGCAGCTACGCTCACAGC | |
| | CAGAGTCTGGACCGTCTCATGAATCCTCTCATCGACCAGTACCTG | |
| | TATTACTTGAGCAGAACAAACACTCCAAGTGGAACCACCACGCAG | |
| | TCAAGGCTTCAGTTTTCTCAGGCCGGAGCGAGTGACATTCGGGAC | |
| | CAGTCTAGGAACTGGCTTCCTGGACCCTGTTACCGCCAGCAGCGA | |
| | GTATCAAAGACATCTGCGGATAACAACAACAGTGAATACTCGTGG | |
| | ACTGGAGCTACCAAGTACCACCTCAATGGCAGAGACTCTCTGGTG | |
| | AATCCGGGCCCGGCCATGGCAAGCCACAAGGACGATGAAGAAAAG | |
| | TTTTTTCCTCAGAGCGGGGTTCTCATCTTTGGGAAGCAAGGCTCA | |
| | GAGAAAACAAATGTGGACATTGAAAAGGTCATGATTACAGACGAA | |
| | GAGGAAATCAGGACAACCAATCCCGTGGCTACGGAGCAGTATGGT | |
| | TCTGTATCTACCAACCTCCAGAGAGGCAACAGACAAGCAGCTACC | |
| | GCAGATGTCAACACACAAGGCGTTCTTCCAGGCATGGTCTGGCAG | |
| | GACAGAGATGTGTACCTTCAGGGGCCCATCTGGGCAAAGATTCCA | |
| | CACACGGACGGACATTTTCACCCCTCTCCCCTCATGGGTGGATTC | |
| | GGACTTAAACACCCTCCTCCACAGATTCTCATCAAGAACACCCCG | |
| | GTACCTGCGAATCCTTCGACCACCTTCAGTGCGGCAAAGTTTGCT | |
| | TCCTTCATCACACAGTACTCCACGGGACAGGTCAGCGTGGAGATC | |
| | GAGTGGGAGCTGCAGAAGGAAAACAGCAAACGCTGGAATCCCGAA | |
| | ATTCAGTACACTTCCAACTACAACAAGTCTGTTAATGTGGACTTT | |
| | ACTGTGGACACTAATGGCGTGTATTCAGAGCCTCGCCCCATTGGC | |
| | ACCAGATACCTGACTCGTAATCTGTAA | |
| AAV5 protein | MSFVDHPPDWLEEVGEGLREFLGLEAGPPKPKPNQQHQDQARGLV | 10 |
| | LPGYNYLGPGNGLDRGEPVNRADEVAREHDISYNEQLEAGDNPYL | |
| | KYNHADAEFQEKLADDTSFGGNLGKAVFQAKKRVLEPFGLVEEGA | |
| | KTAPTGKRIDDHFPKRKKARTEEDSKPSTSSDAEAGPSGSQQLQI | |
| | PAQPASSLGADTMSAGGGGPLGDNNQGADGVGNASGDWHCDSTWM | |
| | GDRVVTKSTRTWVLPSYNNHQYREIKSGSVDGSNANAYFGYSTPW | |
| | GYFDFNRFHSHWSPRDWQRLINNYWGFRPRSLRVKIFNIQVKEVT | |
| | VQDSTTTIANNLTSTVQVFTDDDYQLPYVVGNGTEGCLPAFPPQV | |
| | FTLPQYGYATLNRDNTENPTERSSFFCLEYFPSKMLRTGNNFEFT | |
| | YNFEEVPFHSSFAPSQNLFKLANPLVDQYLYRFVSTNNTGGVQFN | |
| | KNLAGRYANTYKNWFPGPMGRTQGWNLGSGVNRASVSAFATTNRM | |
| | ELEGASYQVPPQPNGMTNNLQGSNTYALENTMIFNSQPANPGTTA | |
| | TYLEGNMLITSESETQPVNRVAYNVGGQMATNNQSSTTAPATGTY | |
| | NLQEIVPGSVWMERDVYLQGPIWAKIPETGAHFHPSPAMGGFGLK | |
| | HPPPMMLIKNTPVPGNITSFSDVPVSSFITQYSTGQVTVEMEWEL | |
| | KKENSKRWNPEIQYTNNYNDPQFVDFAPDSTGEYRTTRPIGTRYL | |
| | TRPL | |
| AAV5 ORF | ATGTCTTTTGTTGATCACCCTCCAGATTGGTTGGAAGAAGTTGGT | 11 |
| | GAAGGTCTTCGCGAGTTTTTGGGCCTTGAAGCGGGCCCACCGAAA | |
| | CCAAAACCCAATCAGCAGCATCAAGATCAAGCCCGTGGTCTTGTG | |
| | CTGCCTGGTTATAACTATCTCGGACCCGGAAACGGTCTCGATCGA | |
| | GGAGAGCCTGTCAACAGGGCAGACGAGGTCGCGCGAGAGCACGAC | |
| | ATCTCGTACAACGAGCAGCTTGAGGCGGGAGACAACCCCTACCTC | |
| | AAGTACAACCACGCGGACGCCGAGTTTCAGGAGAAGCTCGCCGAC | |
| | GACACATCCTTCGGGGGAAACCTCGGAAAGGCAGTCTTTCAGGCC | |
| | AAGAAAAGGGTTCTCGAACCTTTTGGCCTGGTTGAAGAGGGTGCT | |
| | AAGACGGCCCCTACCGGAAAGCGGATAGACGACCACTTTCCAAAA | |
| | AGAAAGAAGGCTCGGACCGAAGAGGACTCCAAGCCTTCCACCTCG | |
| | TCAGACGCCGAAGCTGGACCCAGCGGATCCCAGCAGCTGCAAATC | |
| | CCAGCCCAACCAGCCTCAAGTTTGGGAGCTGATACAATGTCTGCG | |
| | GGAGGTGGCGGCCCATTGGGCGACAATAACCAAGGTGCCGATGGA | |
| | GTGGGCAATGCCTCGGGAGATTGGCATTGCGATTCCACGTGGATG | |
| | GGGGACAGAGTCGTCACCAAGTCCACCCGAACCTGGGTGCTGCCC | |
| | AGCTACAACAACCACCAGTACCGAGAGATCAAAAGCGGCTCCGTC | |
| | GACGGAAGCAACGCCAACGCCTACTTTGGATACAGCACCCCCTGG | |
| | GGGTACTTTGACTTTAACCGCTTCCACAGCCACTGGAGCCCCCGA | |
| | GACTGGCAAAGACTCATCAACAACTACTGGGGCTTCAGACCCCGG | |
| | TCCCTCAGAGTCAAAATCTTCAACATTCAAGTCAAAGAGGTCACG | |
| | GTGCAGGACTCCACCACCACCATCGCCAACAACCTCACCTCCACC | |
| | GTCCAAGTGTTTACGGACGACGACTACCAGCTGCCCTACGTCGTC | |
| | GGCAACGGGACCGAGGGATGCCTGCCGGCCTTCCCTCCGCAGGTC | |
| | TTTACGCTGCCGCAGTACGGTTACGCGACGCTGAACCGCGACAAC | |
| | ACAGAAAATCCCACCGAGAGGAGCAGCTTCTTCTGCCTAGAGTAC | |
| | TTTCCCAGCAAGATGCTGAGAACGGGCAACAACTTTGAGTTTACC | |
| | TACAACTTTGAGGAGGTGCCCTTCCACTCCAGCTTCGCTCCCAGT | |
| | CAGAACCTGTTCAAGCTGGCCAACCCGCTGGTGGACCAGTACTTG | |
| | TACCGCTTCGTGAGCACAAATAACACTGGCGGAGTCCAGTTCAAC | |
| | AAGAACCTGGCCGGGAGATACGCCAACACCTACAAAAACTGGTTC | |
| | CCGGGGCCCATGGGCCGAACCCAGGGCTGGAACCTGGGCTCCGGG | |
| | GTCAACCGCGCCAGTGTCAGCGCCTTCGCCACGACCAATAGGATG | |
| | GAGCTCGAGGGCGCGAGTTACCAGGTGCCCCCGCAGCCGAACGGC | |
| | ATGACCAACAACCTCCAGGGCAGCAACACCTATGCCCTGGAGAAC | |
| | ACTATGATCTTCAACAGCCAGCCGGCGAACCCGGGCACCACCGCC | |
| | ACGTACCTCGAGGGCAACATGCTCATCACCAGCGAGAGCGAGACG | |
| | CAGCCGGTGAACCGCGTGGCGTACAACGTCGGCGGGCAGATGGCC | |
| | ACCAACAACCAGAGCTCCACCACTGCCCCCGCGACCGGCACGTAC | |
| | AACCTCCAGGAAATCGTGCCCGGCAGCGTGTGGATGGAGAGGGAC | |
| | GTGTACCTCCAAGGACCCATCTGGGCCAAGATCCCAGAGACGGGG | |
| | GCGCACTTTCACCCCTCTCCGGCCATGGGCGGATTCGGACTCAAA | |
| | CACCCACCGCCCATGATGCTCATCAAGAACACGCCTGTGCCCGGA | |
| | AATATCACCAGCTTCTCGGACGTGCCCGTCAGCAGCTTCATCACC | |
| | CAGTACAGCACCGGGCAGGTCACCGTGGAGATGGAGTGGGAGCTC | |
| | AAGAAGGAAAACTCCAAGAGGTGGAACCCAGAGATCCAGTACACA | |
| | AACAACTACAACGACCCCCAGTTTGTGGACTTTGCCCCGGACAGC | |
| | ACCGGGGAATACAGAACCACCAGACCTATCGGAACCCGATACCTT | |
| | ACCCGACCCCTTTAA | |
| AAV8 protein | MAADGYLPDWLEDNLSEGIREWWALKPGAPKPKANQQKQDDGRGL | 12 |
| | VLPGYKYLGPFNGLDKGEPVNAADAAALEHDKAYDQQLQAGDNPY | |
| | LRYNHADAEFQERLQEDTSFGGNLGRAVFQAKKRVLEPLGLVEEG | |
| | AKTAPGKKRPVEPSPQRSPDSSTGIGKKGQQPARKRLNFGQTGDS | |
| | ESVPDPQPLGEPPAAPSGVGPNTMAAGGGAPMADNNEGADGVGSS | |
| | SGNWHCDSTWLGDRVITTSTRTWALPTYNNHLYKQISNGTSGGAT | |
| | NDNTYFGYSTPWGYFDFNRFHCHFSPRDWQRLINNNWGFRPKRLS | |
| | FKLFNIQVKEVTQNEGTKTIANNLTSTIQVFTDSEYQLPYVLGSA | |
| | HQGCLPPFPADVFMIPQYGYLTLNNGSQAVGRSSFYCLEYFPSQM | |
| | LRTGNNFQFTYTFEDVPFHSSYAHSQSLDRLMNPLIDQYLYYLSR | |
| | TQTTGGTANTQTLGFSQGGPNTMANQAKNWLPGPCYRQQRVSTTT | |
| | GQNNNSNFAWTAGTKYHLNGRNSLANPGIAMATHKDDEERFFPSN | |
| | GILIFGKQNAARDNADYSDVMLTSEEEIKTTNPVATEEYGIVADN | |
| | LQQQNTAPQIGTVNSQGALPGMVWQNRDVYLQGPIWAKIPHTDGN | |
| | FHPSPLMGGFGLKHPPPQILIKNTPVPADPPTTFNQSKLNSFITQ | |
| | YSTGQVSVEIEWELQKENSKRWNPEIQYTSNYYKSTSVDFAVNTE | |
| | GVYSEPRPIGTRYLTRNL | |
| AAV8 ORF | ATGGCTGCCGATGGTTATCTTCCAGATTGGCTCGAGGACAACCTC | 13 |
| | TCTGAGGGCATTCGCGAGTGGTGGGCGCTGAAACCTGGAGCCCCG | |
| | AAGCCCAAAGCCAACCAGCAAAAGCAGGACGACGGCCGGGGTCTG | |
| | GTGCTTCCTGGCTACAAGTACCTCGGACCCTTCAACGGACTCGAC | |
| | AAGGGGGAGCCCGTCAACGCGGCGGACGCAGCGGCCCTCGAGCAC | |
| | GACAAGGCCTACGACCAGCAGCTGCAGGCGGGTGACAATCCGTAC | |
| | CTGCGGTATAACCACGCCGACGCCGAGTTTCAGGAGCGTCTGCAA | |
| | GAAGATACGTCTTTTGGGGGCAACCTCGGGCGAGCAGTCTTCCAG | |
| | GCCAAGAAGCGGGTTCTCGAACCTCTCGGTCTGGTTGAGGAAGGC | |
| | GCTAAGACGGCTCCTGGAAAGAAGAGACCGGTAGAGCCATCACCC | |
| | CAGCGTTCTCCAGACTCCTCTACGGGCATCGGCAAGAAAGGCCAA | |
| | CAGCCCGCCAGAAAAAGACTCAATTTTGGTCAGACTGGCGACTCA | |
| | GAGTCAGTTCCAGACCCTCAACCTCTCGGAGAACCTCCAGCAGCG | |
| | CCCTCTGGTGTGGGACCTAATACAATGGCTGCAGGCGGTGGCGCA | |
| | CCAATGGCAGACAATAACGAAGGCGCCGACGGAGTGGGTAGTTCC | |
| | TCGGGAAATTGGCATTGCGATTCCACATGGCTGGGCGACAGAGTC | |
| | ATCACCACCAGCACCCGAACCTGGGCCCTGCCCACCTACAACAAC | |
| | CACCTCTACAAGCAAATCTCCAACGGGACATCGGGAGGAGCCACC | |
| | AACGACAACACCTACTTCGGCTACAGCACCCCCTGGGGGTATTTT | |
| | GACTTTAACAGATTCCACTGCCACTTTTCACCACGTGACTGGCAG | |
| | CGACTCATCAACAACAACTGGGGATTCCGGCCCAAGAGACTCAGC | |
| | TTCAAGCTCTTCAACATCCAGGTCAAGGAGGTCACGCAGAATGAA | |
| | GGCACCAAGACCATCGCCAATAACCTCACCAGCACCATCCAGGTG | |
| | TTTACGGACTCGGAGTACCAGCTGCCGTACGTTCTCGGCTCTGCC | |
| | CACCAGGGCTGCCTGCCTCCGTTCCCGGCGGACGTGTTCATGATT | |
| | CCCCAGTACGGCTACCTAACACTCAACAACGGTAGTCAGGCCGTG | |
| | GGACGCTCCTCCTTCTACTGCCTGGAATACTTTCCTTCGCAGATG | |
| | CTGAGAACCGGCAACAACTTCCAGTTTACTTACACCTTCGAGGAC | |
| | GTGCCTTTCCACAGCAGCTACGCCCACAGCCAGAGCTTGGACCGG | |
| | CTGATGAATCCTCTGATTGACCAGTACCTGTACTACTTGTCTCGG | |
| | ACTCAAACAACAGGAGGCACGGCAAATACGCAGACTCTGGGCTTC | |
| | AGCCAAGGTGGGCCTAATACAATGGCCAATCAGGCAAAGAACTGG | |
| | CTGCCAGGACCCTGTTACCGCCAACAACGCGTCTCAACGACAACC | |
| | GGGCAAAACAACAATAGCAACTTTGCCTGGACTGCTGGGACCAAA | |
| | TACCATCTGAATGGAAGAAATTCATTGGCTAATCCTGGCATCGCT | |
| | ATGGCAACACACAAAGACGACGAGGAGCGTTTTTTTCCCAGTAAC | |
| | GGGATCCTGATTTTTGGCAAACAAAATGCTGCCAGAGACAATGCG | |
| | GATTACAGCGATGTCATGCTCACCAGCGAGGAAGAAATCAAAACC | |
| | ACTAACCCTGTGGCTACAGAGGAATACGGTATCGTGGCAGATAAC | |
| | TTGCAGCAGCAAAACACGGCTCCTCAAATTGGAACTGTCAACAGC | |
| | CAGGGGGCCTTACCCGGTATGGTCTGGCAGAACCGGGACGTGTAC | |
| | CTGCAGGGTCCCATCTGGGCCAAGATTCCTCACACGGACGGCAAC | |
| | TTCCACCCGTCTCCGCTGATGGGCGGCTTTGGCCTGAAACATCCT | |
| | CCGCCTCAGATCCTGATCAAGAACACGCCTGTACCTGCGGATCCT | |
| | CCGACCACCTTCAACCAGTCAAAGCTGAACTCTTTCATCACGCAA | |
| | TACAGCACCGGACAGGTCAGCGTGGAAATTGAATGGGAGCTGCAG | |
| | AAGGAAAACAGCAAGCGCTGGAACCCCGAGATCCAGTACACCTCC | |
| | AACTACTACAAATCTACAAGTGTGGACTTTGCTGTTAATACAGAA | |
| | GGCGTGTACTCTGAACCCCGCCCCATTGGCACCCGTTACCTCACC | |
| | CGTAATCTG | |
| AAV9 protein | MAADGYLPDWLEDNLSEGIREWWALKPGAPQPKANQQHQDNARGL | 14 |
| | VLPGYKYLGPGNGLDKGEPVNAADAAALEHDKAYDQQLKAGDNPY | |
| | LKYNHADAEFQERLKEDTSFGGNLGRAVFQAKKRLLEPLGLVEEA | |
| | AKTAPGKKRPVEQSPQEPDSSAGIGKSGAQPAKKRLNFGQTGDTE | |
| | SVPDPQPIGEPPAAPSGVGSLTMASGGGAPVADNNEGADGVGSSS | |
| | GNWHCDSQWLGDRVITTSTRTWALPTYNNHLYKQISNSTSGGSSN | |
| | DNAYFGYSTPWGYFDFNRFHCHFSPRDWQRLINNNWGFRPKRLNF | |
| | KLFNIQVKEVTDNNGVKTIANNLTSTVQVFTDSDYQLPYVLGSAH | |
| | EGCLPPFPADVFMIPQYGYLTLNDGSQAVGRSSFYCLEYFPSQML | |
| | RTGNNFQFSYEFENVPFHSSYAHSQSLDRLMNPLIDQYLYYLSKT | |
| | INGSGQNQQTLKFSVAGPSNMAVQGRNYIPGPSYRQQRVSTTVTQ | |
| | NNNSEFAWPGASSWALNGRNSLMNPGPAMASHKEGEDRFFPLSGS | |
| | LIFGKQGTGRDNVDADKVMITNEEEIKTTNPVATESYGQVATNHQ | |
| | SAQAQAQTGWVQNQGILPGMVWQDRDVYLQGPIWAKIPHTDGNFH | |
| | PSPLMGGFGMKHPPPQILIKNTPVPADPPTAFNKDKLNSFITQYS | |
| | TGQVSVEIEWELQKENSKRWNPEIQYTSNYYKSNNVEFAVNTEGV | |
| | YSEPRPIGTRYLTRNL | |
| AAV9 ORF | ATGGCTGCCGATGGTTATCTTCCAGATTGGCTCGAGGACAACCTT | 15 |
| | AGTGAAGGAATTCGCGAGTGGTGGGCTTTGAAACCTGGAGCCCCT | |
| | CAACCCAAGGCAAATCAACAACATCAAGACAACGCTCGAGGTCTT | |
| | GTGCTTCCGGGTTACAAATACCTTGGACCCGGCAACGGACTCGAC | |
| | AAGGGGGAGCCGGTCAACGCAGCAGACGCGGCGGCCCTCGAGCAC | |
| | GACAAGGCCTACGACCAGCAGCTCAAGGCCGGAGACAACCCGTAC | |
| | CTCAAGTACAACCACGCCGACGCCGAGTTCCAGGAGCGGCTCAAA | |
| | GAAGATACGTCTTTTGGGGGCAACCTCGGGCGAGCAGTCTTCCAG | |
| | GCCAAAAAGAGGCTTCTTGAACCTCTTGGTCTGGTTGAGGAAGCG | |
| | GCTAAGACGGCTCCTGGAAAGAAGAGGCCTGTAGAGCAGTCTCCT | |
| | CAGGAACCGGACTCCTCCGCGGGTATTGGCAAATCGGGTGCACAG | |
| | CCCGCTAAAAAGAGACTCAATTTCGGTCAGACTGGCGACACAGAG | |
| | TCAGTCCCAGACCCTCAACCAATCGGAGAACCTCCCGCAGCCCCC | |
| | TCAGGTGTGGGATCTCTTACAATGGCTTCAGGTGGTGGCGCACCA | |
| | GTGGCAGACAATAACGAAGGTGCCGATGGAGTGGGTAGTTCCTCG | |
| | GGAAATTGGCATTGCGATTCCCAATGGCTGGGGGACAGAGTCATC | |
| | ACCACCAGCACCCGAACCTGGGCCCTGCCCACCTACAACAATCAC | |
| | CTCTACAAGCAAATCTCCAACAGCACATCTGGAGGATCTTCAAAT | |
| | GACAACGCCTACTTCGGCTACAGCACCCCCTGGGGGTATTTTGAC | |
| | TTCAACAGATTCCACTGCCACTTCTCACCACGTGACTGGCAGCGA | |
| | CTCATCAACAACAACTGGGGATTCCGGCCTAAGCGACTCAACTTC | |
| | AAGCTCTTCAACATTCAGGTCAAAGAGGTTACGGACAACAATGGA | |
| | GTCAAGACCATCGCCAATAACCTTACCAGCACGGTCCAGGTCTTC | |
| | ACGGACTCAGACTATCAGCTCCCGTACGTGCTCGGGTCGGCTCAC | |
| | GAGGGCTGCCTCCCGCCGTTCCCAGCGGACGTTTTCATGATTCCT | |
| | CAGTACGGGTATCTGACGCTTAATGATGGAAGCCAGGCCGTGGGT | |
| | CGTTCGTCCTTTTACTGCCTGGAATATTTCCCGTCGCAAATGCTA | |
| | AGAACGGGTAACAACTTCCAGTTCAGCTACGAGTTTGAGAACGTA | |
| | CCTTTCCATAGCAGCTACGCTCACAGCCAAAGCCTGGACCGACTA | |
| | ATGAATCCACTCATCGACCAATACTTGTACTATCTCTCAAAGACT | |
| | ATTAACGGTTCTGGACAGAATCAACAAACGCTAAAATTCAGTGTG | |
| | GCCGGACCCAGCAACATGGCTGTCCAGGGAAGAAACTACATACCT | |
| | GGACCCAGCTACCGACAACAACGTGTCTCAACCACTGTGACTCAA | |
| | AACAACAACAGCGAATTTGCTTGGCCTGGAGCTTCTTCTTGGGCT | |
| | CTCAATGGACGTAATAGCTTGATGAATCCTGGACCTGCTATGGCC | |
| | AGCCACAAAGAAGGAGAGGACCGTTTCTTTCCTTTGTCTGGATCT | |
| | TTAATTTTTGGCAAACAAGGAACTGGAAGAGACAACGTGGATGCG | |
| | GACAAAGTCATGATAACCAACGAAGAAGAAATTAAAACTACTAAC | |
| | CCGGTAGCAACGGAGTCCTATGGACAAGTGGCCACAAACCACCAG | |
| | AGTGCCCAAGCACAGGCGCAGACCGGCTGGGTTCAAAACCAAGGA | |
| | ATACTTCCGGGTATGGTTTGGCAGGACAGAGATGTGTACCTGCAA | |
| | GGACCCATTTGGGCCAAAATTCCTCACACGGACGGCAACTTTCAC | |
| | CCTTCTCCGCTGATGGGAGGGTTTGGAATGAAGCACCCGCCTCCT | |
| | CAGATCCTCATCAAAAACACACCTGTACCTGCGGATCCTCCAACG | |
| | GCCTTCAACAAGGACAAGCTGAACTCTTTCATCACCCAGTATTCT | |
| | ACTGGCCAAGTCAGCGTGGAGATCGAGTGGGAGCTGCAGAAGGAA | |
| | AACAGCAAGCGCTGGAACCCGGAGATCCAGTACACTTCCAACTAT | |
| | TACAAGTCTAATAATGTTGAATTTGCTGTTAATACTGAAGGTGTA | |
| | TATAGTGAACCCCGCCCCATTGGCACCAGATACCTGACTCGTAAT | |
| | CTGT | |
| 5′ ITR | GCGCGCTCGCTCGCTCACTGAGGCCGCCCGGGCAAAGCCCGGGCG | 16 |
| | TCGGGCGACCTTTGGTCGCCCGGCCTCAGTGAGCGAGCGAGCGCG | |
| | CAGAGAGGGAGTGGCCAACTCCATCACTAGGGGTTCCTTGTAGTT | |
| | AATGATTAAC | |
| 3′ ITR | GTTAATCATTAACTACAAGGAACCCCTAGTGATGGAGTTGGCCAC | 17 |
| | TCCCTCTCTGCGCGCTCGCTCGCTCACTGAGGCCGGGCGACCAAA | |
| | GGTCGCCCGACGCCCGGGCTTTGCCCGGGCGGCCTCAGTGAGCGA | |
| | GCGAGCGCGC | |
| BGH poly A | CGACTGTGCCTTCTAGTTGCCAGCCATCTGTTGTTTGCCCCTCCC | 18 |
| Sequence | CCGTGCCTTCCTTGACCCTGGAAGGTGCCACTCCCACTGTCCTTT | |
| | CCTAATAAAATGAGGAAATTGCATCGCATTGTCTGAGTAGGTGTC | |
| | ATTCTATTCTGGGGGGTGGGGTGGGGCAGGACAGCAAGGGGGAGG | |
| | ATTGGGAAGACAATAGCAGGCATGCTGGGGATGCGGTG | |
| ITR | CCTGCAGGCAGCTGCGCGCTCGCTCGCTCACTGAGGCCGCCCGGG | 19 |
| | CAAAGCCCGGGCGTCGGGCGACCTTTGGTCGCCCGGCCTCAGTGA | |
| | GCGAGCGAGCGCGCAGAGAGGGAGTGGCCAACTCCATCACTAGGG | |
| | GTTCCT | |
| ITR | AGGAACCCCTAGTGATGGAGTTGGCCACTCCCTCTCTGCGCGCTC | 20 |
| | GCTCGCTCACTGAGGCCGGGCGACCAAAGGTCGCCCGACGCCCGG | |
| | GCTTTGCCCGGGCGGCCTCAGTGAGCGAGCGAGCGCGCAGCTGCC | |
| | TGCAGG | |
| CMV enhancer | CTAGTTATTAATAGTAATCAATTACGGGGTCATTAGTTCATAGCC | 21 |
| and promoter | CATATATGGAGTTCCGCGTTACATAACTTACGGTAAATGGCCCGC | |
| | CTGGCTGACCGCCCAACGACCCCCGCCCATTGACGTCAATAATGA | |
| | CGTATGTTCCCATAGTAACGCCAATAGGGACTTTCCATTGACGTC | |
| | AATGGGTGGACTATTTACGGTAAACTGCCCACTTGGCAGTACATC | |
| | AAGTGTATCATATGCCAAGTACGCCCCCTATTGACGTCAATGACG | |
| | GTAAATGGCCCGCCTGGCATTATGCCCAGTACATGACCTTATGGG | |
| | ACTTTCCTACTTGGCAGTACATCTACGTATTAGTCATCGCTATTA | |
| | CCATGGTGATGCGGTTTTGGCAGTACATCAATGGGCGTGGATAGC | |
| | GGTTTGACTCACGGGGATTTCCAAGTCTCCACCCCATTGACGTCA | |
| | ATGGGAGTTTGTTTTGGCACCAAAATCAACGGGACTTTCCAAAAT | |
| | GTCGTAACAACTCCGCCCCATTGACGCAAATGGGCGGTAGGCGTG | |
| | TACGGTGGGAGGTCTATATAAGCAGAGCTCTCTGGCTAACTAGAG | |
| | AACCCACTGCTTACTGGCTTATCGAAAT | |
| WPRE | TCCTGTTAATCAACCTCTGGATTACAAAATTTGTGAAAGATTGAC | 22 |
| sequence | TGATATTCTTAACTATGTTGCTCCTTTTACGCTGTGTGGATATGC | |
| | TGCTTTAATGCCTCTGTATCATGCTATTGCTTCCCGTACGGCTTT | |
| | CGTTTTCTCCTCCTTGTATAAATCCTGGTTGCTGTCTCTTTATGA | |
| | GGAGTTGTGGCCCGTTGTCCGTCAACGTGGCGTGGTGTGCTCTGT | |
| | GTTTGCTGACGCAACCCCCACTGGCTGGGGCATTGCCACCACCTG | |
| | TCAACTCCTTTCTGGGACTTTCGCTTTCCCCCTCCCTATCGCCAC | |
| | GGCAGAACTCATCGCCGCCTGCCTTGCCCGCTGCTGGACAGGGGC | |
| | TAGGTTGCTGGGCACTGATAATTCCGTGGTGTTGTCGGGGAAGCT | |
| | GACGTC | |
| DAO1 | GCGCGCTCGCTCGCTCACTGAGGCCGCCCGGGCAAAGCCCGGGCG | 23 |
| expression | TCGGGCGACCTTTGGTCGCCCGGCCTCAGTGAGCGAGCGAGCGCG | |
| cassette | CAGAGAGGGAGTGGCCAACTCCATCACTAGGGGTTCCTTGTAGTT | |
| AAV2 5′ITR | AATGATTAACGGATCTTTACAAATTCAAGCCAGGTGATTTCAACA | |
| (underline) | AATTTTGCTGACGATTTAGGCGCACTATCCCCTAAACTACAAATT | |
| CMV enhancer | AGAAAATAGCGTTCCTTGACACTAGTCTAGTTATTAATAGTAATC | |
| (bold italics) | AATTACGGGGTCATTAGTTCATAGCCCATATATGGAGTTCCGCGT | |
| CMV promoter | TACATAACTTACGGTAAATGGCCCGCCTGGCTGACCGCCCAACGA | |
| (bold | CCCCCGCCCATTGACGTCAATAATGACGTATGTTCCCATAGTAAC | |
| underlined) | GCCAATAGGGACTTTCCATTGACGTCAATGGGTGGACTATTTACG | |
| 5′ untranslated | GTAAACTGCCCACTTGGCAGTACATCAAGTGTATCATATGCCAAG | |
| region | TACGCCCCCTATTGACGTCAATGACGGTAAATGGCCCGCCTGGCA | |
| (underlined | TTATGCCCAGTACATGACCTTATGGGACTTTCCTACTTGGCAGTA | |
| italics) | CATCTACGTATTAGTCATCGCTATTACCATGGTGATGCGGTTTTG | |
| Kozak (italics) | GCAGTACATCAATGGGCGTGGATAGCGGTTTGACTCACGGGGATT | |
| DAO1 ORF | TCCAAGTCTCCACCCCATTGACGTCAATGGGAGTTTGTTTTGGCA | |
| (bold) | CCAAAATCAACGGGACTTTCCAAAATGTCGTAACAACTCCGCCCC | |
| WPRE (italics | ATTGACGCAAATGGGCGGTAGGCGTGTACGGTGGGAGGTCTATAT | |
| lowercase) | AAGCAGAGCTCTCTGGCTAACTAGAGAACCCACTGCTTACTGGCT | |
| BGHpA (bold | TATCGAAATAAGCTTTCTCAGGGGAGATCTCGTTTAGTGAACCGT | |
| lowercase) | CAGATCCTCACTCTCTTCCGCATCGCTGTCTGCGAGGGCCAGCTG | |
| AAV2 3′ITR | TTGGGCTCGCGGTTGAGGACAAACTCTTCGCGGTCTTTCCAGTAC | |
| (bold italics | TCTTGGATCGGAAACCCGTCGGCCTCCGAACGGTACTCCGCCACC | |
| lowercase) | GAGGGACCTGAGCGAGTCCGCATCGACCGGATCGGAAAACCTCTC | |
| | GAGAAAGGCGTCTAACCAGTCACAGTCGCAAGGTAGGCTGAGCAC | |
| | CGTGGCGGGCGGCAGCGGGTGGCGGTCGGGGTTGTTTCTGGCGGA | |
| | GGTGCTGCTGATGATGTAATTAAAGTAGGCGGTCTTGAGACGGCG | |
| | GATGGTCGAGGTGAGGTGTGGCAGGCTTGAGATCCAGCTGTTGGG | |
| | GTGAGTACTCCCTCTCAAAAGCGGGCATTACTTCTGCGCTAAGAT | |
| | TGTCAGTTTCCAAAAACGAGGAGGATTTGATATTCACCTGGCCCG | |
| | ATCTGGCCATACACTTGAGTGACAATGACATCCACTTTGCCTTTC | |
| | TCTCCACAGGTGTCCACTCCCAGGTCCAAGTTTAAACGCCGCCGC | |
| | CATGCCGGCTCTTGGATGGGCCGTTGCCGCGATCCTGATGCTGCA | |
| | GACAGCCATGGCTGAACCCAGCCCAGGCACCCTGCCTAGAAAGGC | |
| | CGGCGTGTTCAGTGATCTGAGCAACCAGGAGCTGAAAGCTGTGCA | |
| | CTCCTTTCTCTGGTCTAAGAAGGAACTGAGACTGCAGCCATCTAG | |
| | TACCACTACAATGGCCAAGAACACCGTGTTCCTGATCGAGATGCT | |
| | GCTGCCCAAGAAATACCACGTGCTGCGGTTCCTGGACAAGGGCGA | |
| | GAGACATCCTGTGCGGGAAGCCAGAGCTGTGATTTTCTTCGGAGA | |
| | TCAGGAGCACCCCAATGTAACCGAATTCGCCGTCGGCCCTCTGCC | |
| | AGGCCCATGCTACATGCGGGCCCTGTCTCCTCGGCCCGGCTACCA | |
| | GAGCTCTTGGGCCAGCAGACCAATCAGCACCGCCGAGTACGCCCT | |
| | GCTGTATCACACACTGCAGGAGGCCACAAAACCTCTGCACCAGTT | |
| | CTTCCTGAACACCACCGGCTTCAGCTTCCAGGACTGCCACGACAG | |
| | ATGTCTGGCCTTCACAGACGTGGCCCCTCGGGGAGTGGCCAGCGG | |
| | CCAACGGAGATCTTGGCTGATCATCCAGAGATACGTCGAGGGATA | |
| | CTTCCTGCACCCTACAGGCCTGGAACTGCTGGTCGACCACGGAAG | |
| | CACCGACGCCGGCCACTGGGCCGTGGAACAAGTGTGGTACAACGG | |
| | CAAATTCTACGGTTCTCCAGAGGAGCTGGCCAGAAAGTACGCCGA | |
| | CGGCGAAGTGGATGTGGTGGTGCTCGAAGATCCTCTGCCTGGCGG | |
| | AAAGGGCCACGATAGCACAGAGGAACCTCCTCTGTTCAGCTCCCA | |
| | TAAGCCCAGAGGCGACTTCCCCTCGCCCATCCACGTGTCCGGCCC | |
| | TAGACTGGTTCAACCTCACGGCCCCAGGTTCCGGCTGGAGGGCAA | |
| | CGCCGTGCTGTACGGCGGCTGGTCCTTCGCCTTCAGACTGAGAAG | |
| | CTCCTCCGGCCTGCAGGTGCTGAACGTGCACTTTGGCGGCGAAAG | |
| | GATCGCTTACGAGGTGTCTGTGCAGGAAGCTGTGGCCCTGTACGG | |
| | CGGCCACACCCCTGCCGGCATGCAGACCAAATACCTGGACGTGGG | |
| | ATGGGGCCTGGGCAGCGTGACACACGAGCTGGCACCTGGCATCGA | |
| | TTGCCCCGAAACAGCTACCTTCCTGGATACCTTTCACTACTACGA | |
| | CGCCGACGACCCCGTCCACTACCCTCGCGCCCTCTGTCTGTTCGA | |
| | GATGCCCACCGGCGTGCCTCTGAGAAGACACTTCAACAGCAACTT | |
| | TAAGGGCGGATTCAACTTCTACGCGGGACTGAAGGGCCAGGTGCT | |
| | GGTGCTGCGCACCACAAGCACCGTCTACAACTATGACTACATCTG | |
| | GGACTTCATCTTCTACCCTAATGGCGTAATGGAAGCCAAGATGCA | |
| | CGCCACCGGCTACGTGCATGCCACGTTTTACACACCTGAAGGACT | |
| | GAGACACGGCACCAGACTGCACACCCACCTGATCGGAAACATCCA | |
| | CACCCACCTGGTGCATTACCGGGTGGACCTGGATGTGGCTGGAAC | |
| | CAAGAACAGCTTCCAGACACTCCAAATGAAGCTGGAAAACATCAC | |
| | AAACCCTTGGAGCCCCAGACACCGGGTGGTGCAGCCTACCCTTGA | |
| | GCAGACCCAGTACAGCTGGGAGAGACAGGCCGCTTTTAGATTTAA | |
| | GAGAAAGCTGCCAAAGTATCTGCTGTTCACCAGCCCTCAGGAGAA | |
| | CCCCTGGGGCCATAAGCGGACCTACAGACTCCAGATCCACAGCAT | |
| | GGCCGACCAGGTGCTTCCTCCTGGCTGGCAGGAGGAACAGGCCAT | |
| | TACCTGGGCTCGGTACCCCCTGGCCGTGACCAAGTACAGAGAGAG | |
| | CGAGCTGTGCAGCAGCTCCATCTACCACCAGAACGACCCTTGGCA | |
| | CCCCCCGGTGGTTTTCGAGCAATTTCTGCACAACAACGAGAATAT | |
| | CGAGAACGAGGATCTGGTGGCATGGGTCACCGTGGGCTTCCTGCA | |
| | CATCCCCCACTCTGAGGACATCCCTAATACCGCCACACCTGGCAA | |
| | TAGCGTGGGCTTTCTGCTGAGACCCTTCAATTTCTTTCCTGAGGA | |
| | CCCTAGCCTGGCCAGCCGGGACACCGTGATCGTGTGGCCTCGGGA | |
| | CAACGGCCCAAACTACGTGCAAAGATGGATCCCCGAAGACAGAGA | |
| | TTGCAGCATGCCTCCTCCTTTCTCCTACAACGGCACATATCGGCC | |
| | TGTGTAGtcctgttaatcaacctctggattacaaaatttgtgaaa | |
| | gattgactgatattcttaactatgttgctccttttacgctgtgtg | |
| | gatatgctgctttaatgcctctgtatcatgctattgcttcccgta | |
| | cggctttcgttttctcctccttgtataaatcctggttgctgtctc | |
| | tttatgaggagttgtggcccgttgtccgtcaacgtggcgtggtgt | |
| | gctctgtgtttgctgacgcaacccccactggctggggcattgcca | |
| | ccacctgtcaactcctttctgggactttcgctttccccctcccta | |
| | tcgccacggcagaactcatcgccgcctgccttgcccgctgctgga | |
| | caggggctaggttgctgggcactgataattccgtggtgttgtcgg | |
| | ggaagctgacgtcCATTGCCACCACCTGTCAACTCCTTTCTGGGA | |
| | CTTTCGCTTTCCCCCTCCCTATCGCCACGGCAGAACTCATCGCCG | |
| | CCTGCCTTGCCCGCTGCTGGACAGGGGCTAGGTTGCTGGGCACTG | |
| | ATAATTCCGTGGTGTTGTCGGGGAAGCTGACGTCcgactgtgcct | |
| | tctagttgccagccatctgttgtttgcccctcccccgtgccttcc | |
| | ttgaccctggaaggtgccactcccactgtcctttcctaataaaat | |
| | gaggaaattgcatcgcattgtctgagtaggtgtcattctattctg | |
| | gggggggggtggggcaggacagcaagggggaggattgggaagaca | |
| | atagcaggcatgctggggatgcggtgggctctatggAGCGCTGGC | |
| | TAGAATTACCTACCGGCCTCCACCATACCTTCGATATTCGCGCCC | |
| | ACTCTCCCATTAATCCGCACAAGTGGATGTGATGCGATTGCCCGC | |
| | TAAGATAgttaatcattaactacaaggaacccctagtgatggagt | |
| | tggccactccctctctgegcgctcgctcgctcactgaggccgggc | |
| | gaccaaaggtcgcccgacgcccgggctttgcccgggeggcctcag | |
| | tgagcgagcgagcgcgc | |
| CMV enhancer | CTAGTTATTAATAGTAATCAATTACGGGGTCATTAGTTCATAGCC | 24 |
| | CATATATGGAGTTCCGCGTTACATAACTTACGGTAAATGGCCCGC | |
| | CTGGCTGACCGCCCAACGACCCCCGCCCATTGACGTCAATAATGA | |
| | CGTATGTTCCCATAGTAACGCCAATAGGGACTTTCCATTGACGTC | |
| | AATGGGTGGACTATTTACGGTAAACTGCCCACTTGGCAGTACATC | |
| | AAGTGTATCATATGCCAAGTACGCCCCCTATTGACGTCAATGACG | |
| | GTAAATGGCCCGCCTGGCATTATGCCCAGTACATGACCTTATGGG | |
| | ACTTTCCTACTTGGCAGTACATCTACGTATTAGTCATCGCTATTA | |
| | CCATG | |
| CMV promoter | GTGATGCGGTTTTGGCAGTACATCAATGGGCGTGGATAGCGGTTT | 25 |
| | GACTCACGGGGATTTCCAAGTCTCCACCCCATTGACGTCAATGGG | |
| | AGTTTGTTTTGGCACCAAAATCAACGGGACTTTCCAAAATGTCGT | |
| | AACAACTCCGCCCCATTGACGCAAATGGGCGGTAGGCGTGTACGG | |
| | TGGGAGGTCTATATAAGCAGAGCTCTCTGGCTAACTAGAGAACCC | |
| | ACTGCTTACTGGCTTATCGAAAT | |
| 5′ untranslated | ACTCTCTTCCGCATCGCTGTCTGCGAGGGCCAGCTGTTGGGCTCG | 26 |
| region | CGGTTGAGGACAAACTCTTCGCGGTCTTTCCAGTACTCTTGGATC | |
| | GGAAACCCGTCGGCCTCCGAACGGTACTCCGCCACCGAGGGACCT | |
| | GAGCGAGTCCGCATCGACCGGATCGGAAAACCTCTCGAGAAAGGC | |
| | GTCTAACCAGTCACAGTCGCAAGGTAGGCTGAGCACCGTGGCGGG | |
| | CGGCAGCGGGTGGCGGTCGGGGTTGTTTCTGGCGGAGGTGCTGCT | |
| | GATGATGTAATTAAAGTAGGCGGTCTTGAGACGGCGGATGGTCGA | |
| | GGTGAGGTGTGGCAGGCTTGAGATCCAGCTGTTGGGGTGAGTACT | |
| | CCCTCTCAAAAGCGGGCATTACTTCTGCGCTAAGATTGTCAGTTT | |
| | CCAAAAACGAGGAGGATTTGATATTCACCTGGCCCGATCTGGCCA | |
| | TACACTTGAGTGACAATGACATCCACTTTGCCTTTCTCTCCACAG | |
| | GTGTCCACTCCCAG | |
| Kozak | GCCGCCGCC | 27 |
| bovine growth | CGACTGTGCCTTCTAGTTGCCAGCCATCTGTTGTTTGCCCCTCCC | 28 |
| hormone pA | CCGTGCCTTCCTTGACCCTGGAAGGTGCCACTCCCACTGTCCTTT | |
| (BGHpA) | CCTAATAAAATGAGGAAATTGCATCGCATTGTCTGAGTAGGTGTC | |
| | AT | |
| DAO1 (AOC1) | TCTATTCTGGGGGGTGGGGTGGGGCAGGACAGCAAGGGGGAGGAT | 29 |
| ORF (codon | TGGGAAGACAATAGCAGGCATGCTGGGGATGCGGTGGGCTCTATG | |
| optimized) | GATGCCGGCTCTTGGATGGGCCGTTGCCGCGATCCTGATGCTGCA | |
| | GACAGCCATGGCTGAACCCAGCCCAGGCACCCTGCCTAGAAAGGC | |
| | CGGCGTGTTCAGTGATCTGAGCAACCAGGAGCTGAAAGCTGTGCA | |
| | CTCCTTTCTCTGGTCTAAGAAGGAACTGAGACTGCAGCCATCTAG | |
| | TACCACTACAATGGCCAAGAACACCGTGTTCCTGATCGAGATGCT | |
| | GCTGCCCAAGAAATACCACGTGCTGCGGTTCCTGGACAAGGGCGA | |
| | GAGACATCCTGTGCGGGAAGCCAGAGCTGTGATTTTCTTCGGAGA | |
| | TCAGGAGCACCCCAATGTAACCGAATTCGCCGTCGGCCCTCTGCC | |
| | AGGCCCATGCTACATGCGGGCCCTGTCTCCTCGGCCCGGCTACCA | |
| | GAGCTCTTGGGCCAGCAGACCAATCAGCACCGCCGAGTACGCCCT | |
| | GCTGTATCACACACTGCAGGAGGCCACAAAACCTCTGCACCAGTT | |
| | CTTCCTGAACACCACCGGCTTCAGCTTCCAGGACTGCCACGACAG | |
| | ATGTCTGGCCTTCACAGACGTGGCCCCTCGGGGAGTGGCCAGCGG | |
| | CCAACGGAGATCTTGGCTGATCATCCAGAGATACGTCGAGGGATA | |
| | CTTCCTGCACCCTACAGGCCTGGAACTGCTGGTCGACCACGGAAG | |
| | CACCGACGCCGGCCACTGGGCCGTGGAACAAGTGTGGTACAACGG | |
| | CAAATTCTACGGTTCTCCAGAGGAGCTGGCCAGAAAGTACGCCGA | |
| | CGGCGAAGTGGATGTGGTGGTGCTCGAAGATCCTCTGCCTGGCGG | |
| | AAAGGGCCACGATAGCACAGAGGAACCTCCTCTGTTCAGCTCCCA | |
| | TAAGCCCAGAGGCGACTTCCCCTCGCCCATCCACGTGTCCGGCCC | |
| | TAGACTGGTTCAACCTCACGGCCCCAGGTTCCGGCTGGAGGGCAA | |
| | CGCCGTGCTGTACGGCGGCTGGTCCTTCGCCTTCAGACTGAGAAG | |
| | CTCCTCCGGCCTGCAGGTGCTGAACGTGCACTTTGGCGGCGAAAG | |
| | GATCGCTTACGAGGTGTCTGTGCAGGAAGCTGTGGCCCTGTACGG | |
| | CGGCCACACCCCTGCCGGCATGCAGACCAAATACCTGGACGTGGG | |
| | ATGGGGCCTGGGCAGCGTGACACACGAGCTGGCACCTGGCATCGA | |
| | TTGCCCCGAAACAGCTACCTTCCTGGATACCTTTCACTACTACGA | |
| | CGCCGACGACCCCGTCCACTACCCTCGCGCCCTCTGTCTGTTCGA | |
| | GATGCCCACCGGCGTGCCTCTGAGAAGACACTTCAACAGCAACTT | |
| | TAAGGGCGGATTCAACTTCTACGCGGGACTGAAGGGCCAGGTGCT | |
| | GGTGCTGCGCACCACAAGCACCGTCTACAACTATGACTACATCTG | |
| | GGACTTCATCTTCTACCCTAATGGCGTAATGGAAGCCAAGATGCA | |
| | CGCCACCGGCTACGTGCATGCCACGTTTTACACACCTGAAGGACT | |
| | GAGACACGGCACCAGACTGCACACCCACCTGATCGGAAACATCCA | |
| | CACCCACCTGGTGCATTACCGGGTGGACCTGGATGTGGCTGGAAC | |
| | CAAGAACAGCTTCCAGACACTCCAAATGAAGCTGGAAAACATCAC | |
| | AAACCCTTGGAGCCCCAGACACCGGGTGGTGCAGCCTACCCTTGA | |
| | GCAGACCCAGTACAGCTGGGAGAGACAGGCCGCTTTTAGATTTAA | |
| | GAGAAAGCTGCCAAAGTATCTGCTGTTCACCAGCCCTCAGGAGAA | |
| | CCCCTGGGGCCATAAGCGGACCTACAGACTCCAGATCCACAGCAT | |
| | GGCCGACCAGGTGCTTCCTCCTGGCTGGCAGGAGGAACAGGCCAT | |
| | TACCTGGGCTCGGTACCCCCTGGCCGTGACCAAGTACAGAGAGAG | |
| | CGAGCTGTGCAGCAGCTCCATCTACCACCAGAACGACCCTTGGCA | |
| | CCCCCCGGTGGTTTTCGAGCAATTTCTGCACAACAACGAGAATAT | |
| | CGAGAACGAGGATCTGGTGGCATGGGTCACCGTGGGCTTCCTGCA | |
| | CATCCCCCACTCTGAGGACATCCCTAATACCGCCACACCTGGCAA | |
| | TAGCGTGGGCTTTCTGCTGAGACCCTTCAATTTCTTTCCTGAGGA | |
| | CCCTAGCCTGGCCAGCCGGGACACCGTGATCGTGTGGCCTCGGGA | |
| | CAACGGCCCAAACTACGTGCAAAGATGGATCCCCGAAGACAGAGA | |
| | TTGCAGCATGCCTCCTCCTTTCTCCTACAACGGCACATATCGGCC | |
| | TGTGTAG | |
| human DAO1 | ATGCCGGCCCTGGGCTGGGCCGTGGCTGCCATCCTGATGCTGCAG | 30 |
| 1 ORF | ACGGCCATGGCGGAGCCCTCCCCGGGGACTCTGCCCAGGAAGGCA | |
| | GGGGTGTTTTCAGACCTAAGCAACCAAGAGCTGAAGGCAGTGCAC | |
| | AGCTTCCTCTGGTCCAAGAAGGAGCTGAGGCTGCAGCCCTCCAGT | |
| | ACCACCACCATGGCCAAGAACACCGTGTTTCTCATCGAGATGCTG | |
| | CTGCCCAAGAAGTACCATGTGCTGAGGTTTCTGGATAAAGGTGAA | |
| | AGGCATCCTGTGCGGGAAGCCCGTGCCGTCATCTTCTTTGGTGAC | |
| | CAGGAGCATCCCAATGTCACCGAGTTTGCTGTGGGGCCCCTGCCA | |
| | GGGCCCTGCTACATGCGAGCACTGTCCCCCAGGCCTGGGTACCAG | |
| | TCCTCCTGGGCATCGAGGCCCATCTCCACAGCAGAGTATGCCCTC | |
| | CTCTACCACACCCTGCAGGAAGCCACCAAGCCCCTGCATCAGTTC | |
| | TTCCTCAATACCACAGGCTTCTCATTCCAAGACTGCCATGACAGA | |
| | TGCCTGGCCTTCACCGATGTGGCCCCCCGGGGTGTGGCTTCTGGC | |
| | CAGCGCCGCAGTTGGCTTATCATACAGCGCTATGTAGAAGGCTAC | |
| | TTTCTGCACCCCACTGGGCTGGAGCTCCTCGTGGATCATGGGAGC | |
| | ACAGATGCTGGGCACTGGGCCGTGGAGCAGGTGTGGTACAACGGG | |
| | AAGTTCTATGGGAGCCCAGAGGAACTGGCTCGGAAGTATGCAGAT | |
| | GGAGAGGTGGACGTGGTGGTCCTGGAGGACCCGCTGCCTGGGGGC | |
| | AAGGGGCATGACAGCACAGAGGAGCCGCCCCTCTTCTCCTCCCAC | |
| | AAGCCCCGCGGGGACTTCCCCAGCCCCATCCATGTGAGCGGCCCC | |
| | CGCTTGGTCCAGCCCCACGGCCCTCGCTTCAGGCTGGAGGGCAAC | |
| | GCTGTGCTCTACGGCGGCTGGAGCTTTGCCTTCCGGCTGCGCTCC | |
| | TCCTCCGGGCTGCAGGTCCTGAACGTGCACTTCGGCGGAGAGCGC | |
| | ATTGCCTATGAGGTCAGCGTGCAAGAGGCAGTGGCGCTGTATGGA | |
| | GGACACACACCTGCAGGCATGCAGACCAAGTACCTCGATGTCGGC | |
| | TGGGGCCTGGGCAGCGTCACTCATGAGTTAGCCCCCGGCATCGAC | |
| | TGCCCGGAGACCGCCACCTTCCTGGACACTTTCCACTACTATGAT | |
| | GCCGATGACCCGGTCCATTATCCCCGAGCCCTCTGCCTCTTTGAA | |
| | ATGCCCACAGGGGTGCCCCTTCGGCGGCACTTTAATTCCAACTTT | |
| | AAAGGTGGCTTCAACTTCTATGCGGGGCTGAAGGGCCAGGTGCTG | |
| | GTGCTGCGGACAACTTCAACTGTCTACAATTATGATTACATTTGG | |
| | GACTTTATCTTCTACCCCAACGGGGTGATGGAGGCCAAGATGCAT | |
| | GCCACTGGCTACGTCCACGCCACCTTCTACACCCCCGAGGGGCTG | |
| | CGCCACGGCACTCGCCTGCACACCCACCTGATTGGCAACATACAC | |
| | ACTCACTTGGTGCACTACCGCGTAGACCTGGATGTGGCAGGCACC | |
| | AAGAACAGCTTCCAGACACTGCAGATGAAGCTAGAAAACATCACC | |
| | AACCCCTGGAGCCCAAGACACCGCGTGGTCCAGCCAACTCTGGAG | |
| | CAGACGCAGTACTCCTGGGAGCGCCAGGCGGCCTTCCGCTTCAAA | |
| | AGGAAGCTGCCTAAGTACCTGCTCTTTACCAGCCCCCAGGAGAAC | |
| | CCCTGGGGCCACAAGCGCACGTACCGCCTGCAGATCCACTCCATG | |
| | GCCGACCAGGTGCTGCCCCCAGGCTGGCAGGAGGAGCAGGCCATC | |
| | ACCTGGGCAAGGACTGAGGGGGGCCAGCCCAGGGCCCTGAGCCAA | |
| | GCTGCTTCGCCTGTGCCTGGCAGGTACCCCCTGGCAGTGACCAAG | |
| | TACCGGGAGTCGGAGCTGTGCAGCAGCAGCATCTACCACCAGAAC | |
| | GACCCCTGGCACCCGCCCGTGGTCTTTGAGCAGTTTCTTCACAAC | |
| | AACGAGAACATTGAAAATGAGGACCTGGTGGCCTGGGTGACGGTG | |
| | GGCTTCCTGCACATCCCCCACTCAGAGGACATTCCCAACACAGCC | |
| | ACACCTGGGAACTCCGTGGGCTTCCTGCTCCGGCCATTCAACTTC | |
| | TTCCCAGAGGACCCCTCCCTGGCATCCAGAGACACTGTGATCGTG | |
| | TGGCCTCGGGACAACGGCCCCAACTACGTCCAGCGCTGGATCCCT | |
| | GAGGACAGGGACTGCTCGATGCCTCCCCCTTTTAGCTACAATGGG | |
| | ACCTATAGACCTGTGTG | |
| human DAO1 | ATGCCGGCCCTGGGCTGGGCCGTGGCTGCCATCCTGATGCTGCAG | 31 |
| 2 ORF | ACGGCCATGGCGGAGCCCTCCCCGGGGACTCTGCCCAGGAAGGCA | |
| | GGGGTGTTTTCAGACCTAAGCAACCAAGAGCTGAAGGCAGTGCAC | |
| | AGCTTCCTCTGGTCCAAGAAGGAGCTGAGGCTGCAGCCCTCCAGT | |
| | ACCACCACCATGGCCAAGAACACCGTGTTTCTCATCGAGATGCTG | |
| | CTGCCCAAGAAGTACCATGTGCTGAGGTTTCTGGATAAAGGTGAA | |
| | AGGCATCCTGTGCGGGAAGCCCGTGCCGTCATCTTCTTTGGTGAC | |
| | CAGGAGCATCCCAATGTCACCGAGTTTGCTGTGGGGCCCCTGCCA | |
| | GGGCCCTGCTACATGCGAGCACTGTCCCCCAGGCCTGGGTACCAG | |
| | TCCTCCTGGGCATCGAGGCCCATCTCCACAGCAGAGTATGCCCTC | |
| | CTCTACCACACCCTGCAGGAAGCCACCAAGCCCCTGCATCAGTTC | |
| | TTCCTCAATACCACAGGCTTCTCATTCCAAGACTGCCATGACAGA | |
| | TGCCTGGCCTTCACCGATGTGGCCCCCCGGGGTGTGGCTTCTGGC | |
| | CAGCGCCGCAGTTGGCTTATCATACAGCGCTATGTAGAAGGCTAC | |
| | TTTCTGCACCCCACTGGGCTGGAGCTCCTCGTGGATCATGGGAGC | |
| | ACAGATGCTGGGCACTGGGCCGTGGAGCAGGTGTGGTACAACGGG | |
| | AAGTTCTATGGGAGCCCAGAGGAACTGGCTCGGAAGTATGCAGAT | |
| | GGAGAGGTGGACGTGGTGGTCCTGGAGGACCCGCTGCCTGGGGGC | |
| | AAGGGGCATGACAGCACAGAGGAGCCGCCCCTCTTCTCCTCCCAC | |
| | AAGCCCCGCGGGGACTTCCCCAGCCCCATCCATGTGAGCGGCCCC | |
| | CGCTTGGTCCAGCCCCACGGCCCTCGCTTCAGGCTGGAGGGCAAC | |
| | GCTGTGCTCTACGGCGGCTGGAGCTTTGCCTTCCGGCTGCGCTCC | |
| | TCCTCCGGGCTGCAGGTCCTGAACGTGCACTTCGGCGGAGAGCGC | |
| | ATTGCCTATGAGGTCAGCGTGCAAGAGGCAGTGGCGCTGTATGGA | |
| | GGACACACACCTGCAGGCATGCAGACCAAGTACCTCGATGTCGGC | |
| | TGGGGCCTGGGCAGCGTCACTCATGAGTTAGCCCCCGGCATCGAC | |
| | TGCCCGGAGACCGCCACCTTCCTGGACACTTTCCACTACTATGAT | |
| | GCCGATGACCCGGTCCATTATCCCCGAGCCCTCTGCCTCTTTGAA | |
| | ATGCCCACAGGGGTGCCCCTTCGGCGGCACTTTAATTCCAACTTT | |
| | AAAGGTGGCTTCAACTTCTATGCGGGGCTGAAGGGCCAGGTGCTG | |
| | GTGCTGCGGACAACTTCAACTGTCTACAATTATGATTACATTTGG | |
| | GACTTTATCTTCTACCCCAACGGGGTGATGGAGGCCAAGATGCAT | |
| | GCCACTGGCTACGTCCACGCCACCTTCTACACCCCCGAGGGGCTG | |
| | CGCCACGGCACTCGCCTGCACACCCACCTGATTGGCAACATACAC | |
| | ACTCACTTGGTGCACTACCGCGTAGACCTGGATGTGGCAGGCACC | |
| | AAGAACAGCTTCCAGACACTGCAGATGAAGCTAGAAAACATCACC | |
| | AACCCCTGGAGCCCAAGACACCGCGTGGTCCAGCCAACTCTGGAG | |
| | CAGACGCAGTACTCCTGGGAGCGCCAGGCGGCCTTCCGCTTCAAA | |
| | AGGAAGCTGCCTAAGTACCTGCTCTTTACCAGCCCCCAGGAGAAC | |
| | CCCTGGGGCCACAAGCGCACGTACCGCCTGCAGATCCACTCCATG | |
| | GCCGACCAGGTGCTGCCCCCAGGCTGGCAGGAGGAGCAGGCCATC | |
| | ACCTGGGCAAGGTACCCCCTGGCAGTGACCAAGTACCGGGAGTCG | |
| | GAGCTGTGCAGCAGCAGCATCTACCACCAGAACGACCCCTGGCAC | |
| | CCGCCCGTGGTCTTTGAGCAGTTTCTTCACAACAACGAGAACATT | |
| | GAAAATGAGGACCTGGTGGCCTGGGTGACGGTGGGCTTCCTGCAC | |
| | ATCCCCCACTCAGAGGACATTCCCAACACAGCCACACCTGGGAAC | |
| | TCCGTGGGCTTCCTGCTCCGGCCATTCAACTTCTTCCCAGAGGAC | |
| | CCCTCCCTGGCATCCAGAGACACTGTGATCGTGTGGCCTCGGGAC | |
| | AACGGCCCCAACTACGTCCAGCGCTGGATCCCTGAGGACAGGGAC | |
| | TGCTCGATGCCTCCCCCTTTTAGCTACAATGGGACCTATAGACCT | |
| | GTGTG | |
| bGH-F3 | ACTCCCACTGTCCTTTCCTAATAAAA | 32 |
| Forward Primer | | |
| bGH-R3 | CCCCCAGAATAGAATGACACCTA | 33 |
| Reverse Primer | | |
| bGH-P3 Probe | TTGCATCGCATTGTCMGBNFQ | 34 |

What is claimed is:

1. A recombinant adeno-associated virus (rAAV) vector comprising an AAV capsid and an expression cassette, the expression cassette comprising a polynucleotide encoding diamine oxidase (DAO1), operatively linked to a promoter.

2. The rAAV vector of claim 1, wherein the polynucleotide encodes an amino acid sequence having at least 95% identity to an amino acid sequence selected from SEQ ID NO: 1 and 2.

3. The rAAV vector of any one of claims 1-2, wherein the polynucleotide comprises a nucleotide sequence having at least 95% identity to a nucleotide sequence selected from SEQ ID NOs: 29-31.

4. The rAAV vector of any one of claims 1-3, wherein the polynucleotide comprises a nucleotide sequence selected from SEQ ID NOs: 29-31.

5. The rAAV vector of any one of claims 1-4, wherein the promoter is a CMV promoter comprising the nucleotide sequence set forth in SEQ ID NO: 21 or a CAG promoter comprising the nucleotide sequence set forth in SEQ ID NO: 5.

6. The rAAV vector of claim 5, wherein the expression cassette comprises the CMV promoter and a CMV enhancer.

7. The rAAV vector of any one of claims 1-6, wherein the expression cassette comprises a polyadenylation (polyA) sequence.

8. The rAAV vector of claim 7, wherein the polyA sequence is a BGH polyA sequence.

9. The rAAV vector of any one of claims 1-8, wherein the expression cassette comprises a Woodchuck Hepatitis Virus Posttranscriptional Regulatory Element (WPRE).

10. The rAAV vector of any one of claims 1-9, wherein the expression cassette comprises a Kozak sequence.

11. A composition comprising an rAAV vector, wherein the rAAV vector comprises:
(a) an AAV capsid, and
(b) an expression cassette, wherein the expression cassette comprises a polynucleotide comprising a nucleotide sequence sharing at least 95% identity to a nucleotide sequence selected from SEQ ID NOs: 29-31, and wherein the polynucleotide is linked to a promoter.

12. The rAAV vector of any one of claims 1-10 or the composition of claim 11, wherein the expression cassette is flanked by two inverted terminal repeats (ITRs).

13. The rAAV vector or the composition of claim 12, wherein the ITRs are AAV2 ITRs.

14. The rAAV vector of any one of claims 1-10 and 12 or the composition of claim 11 or 12, wherein the expression cassette comprises a nucleotide sequence that shares at least 90%, 95%, 96%, 97%, 98%, 99%, or 100% identity to SEQ ID NO: 23.

15. The rAAV vector of any one of claims 1-10 and 12-14 or the composition of any one of claims 11-14, wherein the AAV capsid comprises a VP3 that shares at least 95%, 98%, or 100% identity with AAV2 VP3 (SEQ ID NO: 8), AAV5 VP3 (SEQ ID NO: 10), AAV8 VP3 (SEQ ID NO: 12), or AAV9 VP3 (SEQ ID NO: 14).

16. The rAAV vector of any one of claims 1-10 and 12-15 or the composition of any one of claims 11-15, wherein the AAV capsid comprises a VP3 that shares at least 95%, 98%, or 100% identity with AAV9 VP3 (SEQ ID NO: 14).

17. A composition comprising an rAAV vector, wherein the rAAV vector comprises:
(a) an AAV2, AAV5, AAV8, or AAV9 capsid, and
(b) an expression cassette, wherein the expression cassette comprises a polynucleotide comprising a nucleotide sequence sharing at least 95% identity to a nucleotide sequence selected from SEQ ID NOs: 29-31, and wherein the polynucleotide is linked to a promoter.

18. A composition comprising an rAAV vector, wherein the rAAV vector comprises:
(a) an AAV2, AAV5, AAV8, or AAV9 capsid, and
(b) an expression cassette, wherein the expression cassette comprises a polynucleotide sequence sharing at least 95% identity to SEQ ID NO: 23.

19. The composition of claim 17 or 18, wherein the AAV capsid is AAV2.

20. The composition of claim 17 or 18, wherein the AAV capsid is AAV5.

21. The composition of claim 17 or 18, wherein the AAV capsid is AAV9.

22. A pharmaceutical composition comprising the rAAV vector of any one of claims 1-10 and 12-15 or the composition of any one of claims 11-21, and a pharmaceutically acceptable carrier.

23. The pharmaceutical composition of claim 22, wherein the composition comprises about 1×10⁷ to about 1×10¹⁴ genome copies per milliliter of the rAAV vector.

24. The pharmaceutical composition of claim 22, wherein the composition comprises about 1×10¹² to about 6.2×10¹² genome copies per milliliter of the rAAV vector.

25. The pharmaceutical composition of any one of claims 22-24, wherein the composition is formulated for administration into the lacrimal gland.

26. The pharmaceutical composition of any one of claims 22-25, wherein the composition is formulated for administration onto the ocular surface.

27. The pharmaceutical composition of any one of claims 22-26, wherein the composition is formulated for use, or adaptable for use, in the treatment of an ocular disease, disorder, or condition.

28. A method of treating a condition in a subject in need thereof, the method comprising administering an effective amount of the pharmaceutical composition of any one of claims 22-27 to the eye of the subject.

29. The method of claim 28, wherein the pharmaceutical composition is delivered to an ocular secretory gland of the subject.

30. The method of claim 28 or 29, wherein the pharmaceutical composition is delivered to the lacrimal gland.

31. The method of claim 30, wherein cells within the lacrimal gland are transduced by the rAAV vector.

32. The method of claim 31, wherein the transduced cells within the lacrimal gland express an effective amount of DAO1 into the tear film and optionally onto the ocular surface of the subject.

33. The method of any one of claims 28-32, wherein the pharmaceutical composition is delivered to an accessory lacrimal gland.

34. The method of any one of claims 28-33, wherein about 1×10⁹ to about 1×10¹⁰, about 1×10¹⁰ to about 1×10¹¹, about 1×10¹¹ to about 1×10¹², about 1×10¹² to about 1×10¹³, or about 1×10¹³ to about 1×10¹⁵ genome copies of the rAAV vector are administered.

35. The method of any one of claims 28-34, wherein the condition is an optical condition.

36. The method of any one of claims 28-35, wherein the condition is associated with increased histamine production and/or increased histamine signaling.

37. The method of any one of claims 28-36, wherein the condition is an inflammatory condition, optionally an inflammatory condition of the eye.

38. The method of any one of claims 28-37, wherein the condition is an autoimmune condition.

39. The method of any one of claims 28-37, wherein the condition is an allergic condition.

40. The method of claim 39, wherein the condition is an allergic reaction to a therapeutic agent.

41. The method of claim 39, wherein the condition is an allergic reaction to a microbial agent.

42. The method of any one of claims 28-37, wherein the condition comprises vernal keratoconjunctivitis.

43. The method of any one of claims 28-37, wherein the condition comprises atopic keratoconjunctivitis.

44. The method of any one of claims 28-37, wherein the condition comprises seasonal or perennial allergic conjunctivitis.

45. The method of any one of claims 28-44, wherein the administration results in expression of DAO1 in the cells of the lacrimal gland and/or an accessory lacrimal gland.

46. The method of any one of claims 28-45, wherein the administration results in secretion of DAO1 into the tear film.

47. The method of claim 46, wherein secretion of DAO1 into the tear film is stimulated by a cholinergic agonist.

48. The method of any one of claims 28-47, wherein the administration results in an improvement of one or more symptoms of the condition.

49. The method of claim 48, wherein the symptom is selected from the group consisting of itching, swelling, tearing, and redness.

50. The method of any one of claims 28-49, wherein the administration results in an improvement of 0.5 points, 1 point, 1.5 points, 2 points, 2.5 points, 3 point, 3.5 points, or 4 points on the Conjunctival Itching Grading Scale.

51. The method of any one of claims 28-50, wherein the administration results in an improvement of 1 point, 2 points, 3 point, or 4 points on the Conjunctival Redness Assessment Grading Scale.

52. The method of any one of claims 28-51, wherein the administration results in an improvement of about 10% to about 20%, about 20% to about 30%, about 30% to about 40%, about 40% to about 50%, about 50% to about 60%, about 60% to about 70%, about 70% to about 80%, about 80% to about 90%, or more than about 95% on the Schirmer Test.

53. The method of any one of claims 28-52, wherein the administration results in an improvement of about 10% to about 20%, about 20% to about 30%, about 30% to about 40%, about 40% to about 50%, about 50% to about 60%, about 60% to about 70%, about 70% to about 80%, about 80% to about 90%, or more than about 95% in at least one area on the Corneal Staining Grading Scale.

54. The method of any one of claims 48-53, wherein the improvement is measured about 1 months, about 2 months, about 3 months, about 4 months, about 5 months, about 6 months, about 9 months, or about 12 months after the administration.

55. The method of any one of claims 48-54, wherein the improvement persists for at least 3 months, at least 6 months, at least 9 months, at least 12 months, at least 15 months, at least 18 months, at least 24 months, at least 3 years, at least 4 years, or at least 5 years after the administration.

56. The method of any one of claims 28-55, further comprising administering one or more additional therapeutic agents to the subject.

57. The method of any one of claims 28-56, wherein the subject is human.

58. The pharmaceutical composition of any one of claims 22-27 for use in a method of treating a condition in a subject in need thereof comprising administering an effective amount of the pharmaceutical composition to the eye of the subject.

59. The pharmaceutical composition of any one of claims 22-27 for use in the manufacture of a medicament for treating a condition in a subject in need thereof.

60. A kit comprising a pharmaceutical composition of rAAV vector of any one of claims 1-10 and 12-16 or the composition of any one of claims 11-21, and a pharmaceutically acceptable carrier, and instructions for use in treating a condition in a subject, comprising administering the pharmaceutical composition to the eye of the subject.

61. A kit comprising a pharmaceutical composition of rAAV vector of any one of claims 1-10 and 12-16 or the composition of any one of claims 11-21, and a pharmaceutically acceptable carrier, and instructions for use in treating a condition associated with histamine production and/or increased histamine signaling in a subject, comprising administering the pharmaceutical composition to the eye of the subject.

62. A kit comprising a pharmaceutical composition of rAAV vector of any one of claims 1-10 and 12-16 or the composition of any one of claims 11-21, and a pharmaceutically acceptable carrier, and instructions for use in treating an autoimmune condition in a subject, comprising administering the pharmaceutical composition to the eye of the subject.

63. A kit comprising a pharmaceutical composition of rAAV vector of any one of claims 1-10 and 12-16 or the composition of any one of claims 11-21, and a pharmaceutically acceptable carrier, and instructions for use in treating an allergy condition in a subject, comprising administering the pharmaceutical composition to the eye of the subject.

64. A pharmaceutical composition, comprising
a) a polypeptide comprising a diamine oxidase (“DAO1”) enzyme, or a fragment thereof, optionally wherein the polypeptide has an amino acid sequence having at least 90% identity to an amino acid sequence selected from SEQ ID NO: 1 and 2; and
b) a pharmaceutically-acceptable carrier suitable for administration to an eye of a human subject.

65. A pharmaceutical composition, comprising
a) a vector comprising a polynucleotide that encodes a polypeptide comprising a diamine oxidase (“DAO1”) enzyme, or a fragment thereof, optionally wherein the polypeptide has an amino acid sequence having at least 90% identity to an amino acid sequence selected from SEQ ID NO: 1 and 2; and
b) a pharmaceutically-acceptable carrier suitable for administration to an eye of a human subject.

66. The pharmaceutical composition of claim 64 or 65, wherein the pharmaceutically-acceptable carrier comprises water; sterile water; pyrogen-free water; phosphate-buffered saline; HEPES-buffered saline; an isotonic sodium chloride solution; a balanced salt solution; a wetting agent; a surfactant; a tonicity agent; a pH modifier; a viscosity-modifying agent; a buffering agent; a disaccharide, optionally, sucrose or trehalose; a cellulose and/or a derivate thereof; an amino acid, optionally histidine; or any combination thereof.

67. The pharmaceutical composition of any one of claims 64-66, wherein the polypeptide has at least 91, 92, 93, 94, 95, 96, 97, 98, 99, or 100% sequence identity to SEQ ID NO: 1 or SEQ ID NO: 2.

68. The pharmaceutical composition of any one of claims 64-67, wherein the polypeptide comprises at least 20, 40, 60, 80, 100, 120, 140, 160, 180, 200, 220, 240, 260, 280, 300, 320, 340, 360, 380, 400, 420, 440, 460, 480, or 500 amino acids.

69. The pharmaceutical composition of any one of claims 64-68, wherein the formulation is a liquid formulated for application to an ocular surface, or into an ocular surface, or for intralacrimal injection, of the eye of the human subject.

70. The pharmaceutical composition of any one of claims 65-69, comprising the vector, wherein the vector is present in the composition in an amount effective to express from 100 μg/mL to 50 μg/mL of the polypeptide in a tear film of a subject subsequent to administration of the composition to the subject.

71. The pharmaceutical composition of any one of claims 65-70, comprising the vector, wherein the polynucleotide is operably linked to a promotor.

72. The pharmaceutical composition of any one of claims 65-71, comprising the vector, wherein the vector is engineered to constitutively express the polypeptide having an amino acid sequence having at least 90% identity to an amino acid sequence selected from SEQ ID NO: 1 and 2.

73. The pharmaceutical composition of any one of claims 65-72, comprising the vector, wherein the vector comprises a virus, optionally an adenoviral vector or a lentiviral vector; a plasmid; an episome; or an artificial chromosome; and optionally comprises one or more lipids, polycations, DNA-carrier proteins, histones, pseudocapsids, chimeric proteins, or endocytosis receptor proteins.

74. The pharmaceutical composition of any one of claim 64, or 66-69, comprising the polypeptide, wherein the polypeptide is present in the pharmaceutical composition at a concentration of 100 μg/mL to 50 μg/mL.

75. The pharmaceutical composition of any one of claims 64, 66-69, or 74, comprising the polypeptide, wherein the polypeptide is present in the pharmaceutical composition in an amount of 500 ng to 5 μg.

76. The pharmaceutical composition of any one of claims 64, 66-69, 74, or 75, comprising the polypeptide, wherein the polypeptide is present in the pharmaceutical composition in a unit dose amount.

77. A method of treating an ocular disease, disorder, or condition in a subject in need thereof, the method comprising administering an effective amount of the pharmaceutical composition of any one of claims 64-76 to an eye of the subject.

78. The method of claim 77, wherein the ocular disease, disorder, or condition is associated with increased histamine production and/or increased histamine signaling.

79. The method of claim 77 or 78, wherein the condition is an inflammatory condition, optionally an inflammatory condition of the eye.

80. The method of any one of claims 77-79, wherein the condition is an autoimmune condition.

81. The method of any one of claims 77-80, wherein the condition is an allergic condition.

82. The method of any one of claims 77-81, wherein the condition is an allergic reaction to a therapeutic agent.

83. The method of any one of claims 77-82, wherein the condition is an allergic reaction to a microbial agent.

84. The method of any one of claims 77-83, wherein the condition comprises vernal keratoconjunctivitis.

85. The method of any one of claims 77-84, wherein the condition comprises atopic keratoconjunctivitis.

86. The method of any one of claims 77-85, wherein the condition comprises seasonal or perennial allergic conjunctivitis.

87. The method of any one of claims 77-86, wherein the administration results in expression of a functional diamine oxidase in one or more cells of a lacrimal gland and/or an accessory lacrimal gland of the subject.

88. The method of any one of claims 77-87, wherein the administration results in secretion of a functional diamine oxidase into a tear film of the subject.

89. The method of claim 88, wherein secretion of the functional diamine oxidase into the tear film is stimulated by a cholinergic agonist.

90. The method of any one of claims 77-89, wherein the administration is to an ocular surface, or into an ocular surface of the subject, and/or to a lacrimal gland of the subject.

91. The method of any one of claims 77-90, wherein the symptom is selected from the group consisting of itching, swelling, tearing, and redness.

92. The method of any one of claims 77-91, wherein the administration results in an improvement of 0.5 points, 1 point, 1.5 points, 2 points, 2.5 points, 3 point, 3.5 points, or 4 points on the Conjunctival Itching Grading Scale.

93. The method of any one of claims 77-92, wherein the subject is a human subject.

94. A kit comprising the pharmaceutical composition of any one of claims 64-76 and instructions for use in treating a condition in a human subject, comprising administering the pharmaceutical composition to an eye of the human subject.

95. The pharmaceutical composition of any one of claims 64-76 for use in the manufacture of a medicament for treating a condition in a human subject in need thereof.


SEQUENCE LISTING (34 sequences)

SEQ ID NO: 1 - protein, 770 aa, Homo sapiens
MPALGWAVAAILMLQTAMAEPSPGTLPRKAGVFSDLSNQELKAVHSFLWSKKELRLQPSSTTTMAKNTVFLIEMLLPKKYHVLRFLDKGERHPVREARAVIFFGDQEHPNVTEFAVGPLPGPCYMRALSPRPGYQSSWASRPISTAEYALLYHTLQEATKPLHQFFLNTTGFSFQDCHDRCLAFTDVAPRGVASGQRRSWLIIQRYVEGYFLHPTGLELLVDHGSTDAGHWAVEQVWYNGKFYGSPEELARKYADGEVDVVVLEDPLPGGKGHDSTEEPPLFSSHKPRGDFPSPIHVSGPRLVQPHGPRFRLEGNAVLYGGWSFAFRLRSSSGLQVLNVHFGGERIAYEVSVQEAVALYGGHTPAGMQTKYLDVGWGLGSVTHELAPGIDCPETATFLDTFHYYDADDPVHYPRALCLFEMPTGVPLRRHFNSNFKGGFNFYAGLKGQVLVLRTTSTVYNYDYIWDFIFYPNGVMEAKMHATGYVHATFYTPEGLRHGTRLHTHLIGNIHTHLVHYRVDLDVAGTKNSFQTLQMKLENITNPWSPRHRVVQPTLEQTQYSWERQAAFRFKRKLPKYLLFTSPQENPWGHKRTYRLQIHSMADQVLPPGWQEEQAITWARTEGGQPRALSQAASPVPGRYPLAVTKYRESELCSSSIYHQNDPWHPPVVFEQFLHNNENIENEDLVAWVTVGFLHIPHSEDIPNTATPGNSVGFLLRPFNFFPEDPSLASRDTVIVWPRDNGPNYVQRWIPEDRDCSMPPPFSYNGTYRPV

SEQ ID NO: 2 - protein, 751 aa, Homo sapiens
MPALGWAVAAILMLQTAMAEPSPGTLPRKAGVFSDLSNQELKAVHSFLWSKKELRLQPSSTTTMAKNTVFLIEMLLPKKYHVLRFLDKGERHPVREARAVIFFGDQEHPNVTEFAVGPLPGPCYMRALSPRPGYQSSWASRPISTAEYALLYHTLQEATKPLHQFFLNTTGFSFQDCHDRCLAFTDVAPRGVASGQRRSWLIIQRYVEGYFLHPTGLELLVDHGSTDAGHWAVEQVWYNGKFYGSPEELARKYADGEVDVVVLEDPLPGGKGHDSTEEPPLFSSHKPRGDFPSPIHVSGPRLVQPHGPRFRLEGNAVLYGGWSFAFRLRSSSGLQVLNVHFGGERIAYEVSVQEAVALYGGHTPAGMQTKYLDVGWGLGSVTHELAPGIDCPETATFLDTFHYYDADDPVHYPRALCLFEMPTGVPLRRHFNSNFKGGFNFYAGLKGQVLVLRTTSTVYNYDYIWDFIFYPNGVMEAKMHATGYVHATFYTPEGLRHGTRLHTHLIGNIHTHLVHYRVDLDVAGTKNSFQTLQMKLENITNPWSPRHRVVQPTLEQTQYSWERQAAFRFKRKLPKYLLFTSPQENPWGHKRTYRLQIHSMADQVLPPGWQEEQAITWARYPLAVTKYRESELCSSSIYHQNDPWHPPVVFEQFLHNNENIENEDLVAWVTVGFLHIPHSEDIPNTATPGNSVGFLLRPFNFFPEDPSLASRDTVIVWPRDNGPNYVQRWIPEDRDCSMPPPFSYNGTYRPV

SEQ ID NO: 3 - DNA, 2666 bp, Homo sapiens
  source 1..2666: mol_type = genomic DNA
gcaaaggctggaagcagagcgaactgggagcagagcacacagagccgtggagcgagagatgccggccctgggctgggccgtggctgccatcctgatgctgcagacggccatggcggagccctccccggggactctgcccaggaaggcaggggtgttttcagacctaagcaaccaagagctgaaggcagtgcacagcttcctctggtccaagaaggagctgaggctgcagccctccagtaccaccaccatggccaagaacaccgtgtttctcatcgagatgctgctgcccaagaagtaccatgtgctgaggtttctggataaaggtgaaaggcatcctgtgcgggaagcccgtgccgtcatcttctttggtgaccaggagcatcccaatgtcaccgagtttgctgtggggcccctgccagggccctgctacatgcgagcactgtcccccaggcctgggtaccagtcctcctgggcatcgaggcccatctccacagcagagtatgccctcctctaccacaccctgcaggaagccaccaagcccctgcatcagttcttcctcaataccacaggcttctcattccaagactgccatgacagatgcctggccttcaccgatgtggccccccggggtgtggcttctggccagcgccgcagttggcttatcatacagcgctatgtagaaggctactttctgcaccccactgggctggagctcctcgtggatcatgggagcacagatgctgggcactgggccgtggagcaggtgtggtacaacgggaagttctatgggagcccagaggaactggctcggaagtatgcagatggagaggtggacgtggtggtcctggaggacccgctgcctgggggcaaggggcatgacagcacagaggagccgcccctcttctcctcccacaagccccgcggggacttccccagccccatccatgtgagcggcccccgcttggtccagccccacggccctcgcttcaggctggagggcaacgctgtgctctacggcggctggagctttgccttccggctgcgctcctcctccgggctgcaggtcctgaacgtgcacttcggcggagagcgcattgcctatgaggtcagcgtgcaagaggcagtggcgctgtatggaggacacacacctgcaggcatgcagaccaagtacctcgatgtcggctggggcctgggcagcgtcactcatgagttagcccccggcatcgactgcccggagaccgccaccttcctggacactttccactactatgatgccgatgacccggtccattatccccgagccctctgcctctttgaaatgcccacaggggtgccccttcggcggcactttaattccaactttaaaggtggcttcaacttctatgcggggctgaagggccaggtgctggtgctgcggacaacttcaactgtctacaattatgattacatttgggactttatcttctaccccaacggggtgatggaggccaagatgcatgccactggctacgtccacgccaccttctacacccccgaggggctgcgccacggcactcgcctgcacacccacctgattggcaacatacacactcacttggtgcactaccgcgtagacctggatgtggcaggcaccaagaacagcttccagacactgcagatgaagctagaaaacatcaccaacccctggagcccaagacaccgcgtggtccagccaactctggagcagacgcagtactcctgggagcgccaggcggccttccgcttcaaaaggaagctgcctaagtacctgctctttaccagcccccaggagaacccctggggccacaagcgcacgtaccgcctgcagatccactccatggccgaccaggtgctgcccccaggctggcaggaggagcaggccatcacctgggcaaggactgaggggggccagcccagggccctgagccaagctgcttcgcctgtgcctggcaggtaccccctggcagtgaccaagtaccgggagtcggagctgtgcagcagcagcatctaccaccagaacgacccctggcacccgcccgtggtctttgagcagtttcttcacaacaacgagaacattgaaaatgaggacctggtggcctgggtgacggtgggcttcctgcacatcccccactcagaggacattcccaacacagccacacctgggaactccgtgggcttcctgctccggccattcaacttcttcccagaggacccctccctggcatccagagacactgtgatcgtgtggcctcgggacaacggccccaactacgtccagcgctggatccctgaggacagggactgctcgatgcctcccccttttagctacaatgggacctatagacctgtgtgaccagcccccagttcctcccccagttcctcccaggaagcccaggagcctcactggggcagacaataaaccctcagagcctcgctctgtgtgctgcttcttgcggggaggcacagggccatgtgtgtaggaaacacacgaacagacgtgcacacacacagacgtgcacacacacacagacatgcacacacacacagacgtgcacacacacagacgtgcacgcactcacacggacatgcacacacatggcatgtactttattcacactggtctactgcagtccagaaaagccaccatt

SEQ ID NO: 4 - DNA, 2609 bp, Homo sapiens
  source 1..2609: mol_type = genomic DNA
gcaaaggctggaagcagagcgaactgggagcagagcacacagagccgtggagcgagagatgccggccctgggctgggccgtggctgccatcctgatgctgcagacggccatggcggagccctccccggggactctgcccaggaaggcaggggtgttttcagacctaagcaaccaagagctgaaggcagtgcacagcttcctctggtccaagaaggagctgaggctgcagccctccagtaccaccaccatggccaagaacaccgtgtttctcatcgagatgctgctgcccaagaagtaccatgtgctgaggtttctggataaaggtgaaaggcatcctgtgcgggaagcccgtgccgtcatcttctttggtgaccaggagcatcccaatgtcaccgagtttgctgtggggcccctgccagggccctgctacatgcgagcactgtcccccaggcctgggtaccagtcctcctgggcatcgaggcccatctccacagcagagtatgccctcctctaccacaccctgcaggaagccaccaagcccctgcatcagttcttcctcaataccacaggcttctcattccaagactgccatgacagatgcctggccttcaccgatgtggccccccggggtgtggcttctggccagcgccgcagttggcttatcatacagcgctatgtagaaggctactttctgcaccccactgggctggagctcctcgtggatcatgggagcacagatgctgggcactgggccgtggagcaggtgtggtacaacgggaagttctatgggagcccagaggaactggctcggaagtatgcagatggagaggtggacgtggtggtcctggaggacccgctgcctgggggcaaggggcatgacagcacagaggagccgcccctcttctcctcccacaagccccgcggggacttccccagccccatccatgtgagcggcccccgcttggtccagccccacggccctcgcttcaggctggagggcaacgctgtgctctacggcggctggagctttgccttccggctgcgctcctcctccgggctgcaggtcctgaacgtgcacttcggcggagagcgcattgcctatgaggtcagcgtgcaagaggcagtggcgctgtatggaggacacacacctgcaggcatgcagaccaagtacctcgatgtcggctggggcctgggcagcgtcactcatgagttagcccccggcatcgactgcccggagaccgccaccttcctggacactttccactactatgatgccgatgacccggtccattatccccgagccctctgcctctttgaaatgcccacaggggtgccccttcggcggcactttaattccaactttaaaggtggcttcaacttctatgcggggctgaagggccaggtgctggtgctgcggacaacttcaactgtctacaattatgattacatttgggactttatcttctaccccaacggggtgatggaggccaagatgcatgccactggctacgtccacgccaccttctacacccccgaggggctgcgccacggcactcgcctgcacacccacctgattggcaacatacacactcacttggtgcactaccgcgtagacctggatgtggcaggcaccaagaacagcttccagacactgcagatgaagctagaaaacatcaccaacccctggagcccaagacaccgcgtggtccagccaactctggagcagacgcagtactcctgggagcgccaggcggccttccgcttcaaaaggaagctgcctaagtacctgctctttaccagcccccaggagaacccctggggccacaagcgcacgtaccgcctgcagatccactccatggccgaccaggtgctgcccccaggctggcaggaggagcaggccatcacctgggcaaggtaccccctggcagtgaccaagtaccgggagtcggagctgtgcagcagcagcatctaccaccagaacgacccctggcacccgcccgtggtctttgagcagtttcttcacaacaacgagaacattgaaaatgaggacctggtggcctgggtgacggtgggcttcctgcacatcccccactcagaggacattcccaacacagccacacctgggaactccgtgggcttcctgctccggccattcaacttcttcccagaggacccctccctggcatccagagacactgtgatcgtgtggcctcgggacaacggccccaactacgtccagcgctggatccctgaggacagggactgctcgatgcctcccccttttagctacaatgggacctatagacctgtgtgaccagcccccagttcctcccccagttcctcccaggaagcccaggagcctcactggggcagacaataaaccctcagagcctcgctctgtgtgctgcttcttgcggggaggcacagggccatgtgtgtaggaaacacacgaacagacgtgcacacacacagacgtgcacacacacacagacatgcacacacacacagacgtgcacacacacagacgtgcacgcactcacacggacatgcacacacatggcatgtactttattcacactggtctactgcagtccagaaaagccaccatt

SEQ ID NO: 5 - DNA, 1701 bp, Synthetic construct
  misc_feature 1..1701: note = CAG Promoter
  source 1..1701: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaa

SEQ ID NO: 6 - protein, 735 aa, Adeno-associated dependoparvovirus A [sequence omitted]

SEQ ID NO: 7 - DNA, 2208 bp, Adeno-associated dependoparvovirus A
  source 1..2208: mol_type = genomic DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 8 - protein, 533 aa, Adeno-associated dependoparvovirus A
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 9 - DNA, 1602 bp, Adeno-associated dependoparvovirus A
  source 1..1602: mol_type = genomic DNA
atggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 10 - protein, 724 aa, Adeno-associated dependoparvovirus A
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 11 - DNA, 2175 bp, Adeno-associated dependoparvovirus A
  source 1..2175: mol_type = genomic DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 12 - protein, 738 aa, Adeno-associated dependoparvovirus A
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 13 - DNA, 2214 bp, Adeno-associated dependoparvovirus A
  source 1..2214: mol_type = genomic DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 14 - protein, 736 aa, Adeno-associated dependoparvovirus A
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - DNA, 2209 bp, Adeno-associated dependoparvovirus A
  source 1..2209: mol_type = genomic DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgt

SEQ ID NO: 16 - DNA, 145 bp, Synthetic construct
  misc_feature 1..145: note = 5' ITR
  source 1..145: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaac

SEQ ID NO: 17 - DNA, 145 bp, Synthetic construct
  misc_feature 1..145: note = 3' ITR
  source 1..145: mol_type = other DNA
gttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 18 - DNA, 218 bp, Synthetic construct
  misc_feature 1..218: note = BGH polyA Sequence
  source 1..218: mol_type = other DNA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtg

SEQ ID NO: 19 - DNA, 141 bp, Synthetic construct
  misc_feature 1..141: note = ITR
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 20 - DNA, 141 bp, Synthetic construct
  misc_feature 1..141: note = ITR
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 21 - DNA, 613 bp, Synthetic construct
  misc_feature 1..613: note = CMV promoter
  source 1..613: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaat

SEQ ID NO: 22 - DNA, 411 bp, Synthetic construct
  misc_feature 1..411: note = WPRE sequence
  source 1..411: mol_type = other DNA
tcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctccctatcgccacggcagaactcatcgccgcctgccttgcccgctgctggacaggggctaggttgctgggcactgataattccgtggtgttgtcggggaagctgacgtc

SEQ ID NO: 23 - DNA, 4743 bp, Synthetic construct
  misc_feature 1..4743: note = DAO1 expression cassette
  source 1..4743: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacggatctttacaaattcaagccaggtgatttcaacaaattttgctgacgatttaggcgcactatcccctaaactacaaattagaaaatagcgttccttgacactagtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaataagctttctcaggggagatctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacgccgccgccatgccggctcttggatgggccgttgccgcgatcctgatgctgcagacagccatggctgaacccagcccaggcaccctgcctagaaaggccggcgtgttcagtgatctgagcaaccaggagctgaaagctgtgcactcctttctctggtctaagaaggaactgagactgcagccatctagtaccactacaatggccaagaacaccgtgttcctgatcgagatgctgctgcccaagaaataccacgtgctgcggttcctggacaagggcgagagacatcctgtgcgggaagccagagctgtgattttcttcggagatcaggagcaccccaatgtaaccgaattcgccgtcggccctctgccaggcccatgctacatgcgggccctgtctcctcggcccggctaccagagctcttgggccagcagaccaatcagcaccgccgagtacgccctgctgtatcacacactgcaggaggccacaaaacctctgcaccagttcttcctgaacaccaccggcttcagcttccaggactgccacgacagatgtctggccttcacagacgtggcccctcggggagtggccagcggccaacggagatcttggctgatcatccagagatacgtcgagggatacttcctgcaccctacaggcctggaactgctggtcgaccacggaagcaccgacgccggccactgggccgtggaacaagtgtggtacaacggcaaattctacggttctccagaggagctggccagaaagtacgccgacggcgaagtggatgtggtggtgctcgaagatcctctgcctggcggaaagggccacgatagcacagaggaacctcctctgttcagctcccataagcccagaggcgacttcccctcgcccatccacgtgtccggccctagactggttcaacctcacggccccaggttccggctggagggcaacgccgtgctgtacggcggctggtccttcgccttcagactgagaagctcctccggcctgcaggtgctgaacgtgcactttggcggcgaaaggatcgcttacgaggtgtctgtgcaggaagctgtggccctgtacggcggccacacccctgccggcatgcagaccaaatacctggacgtgggatggggcctgggcagcgtgacacacgagctggcacctggcatcgattgccccgaaacagctaccttcctggatacctttcactactacgacgccgacgaccccgtccactaccctcgcgccctctgtctgttcgagatgcccaccggcgtgcctctgagaagacacttcaacagcaactttaagggcggattcaacttctacgcgggactgaagggccaggtgctggtgctgcgcaccacaagcaccgtctacaactatgactacatctgggacttcatcttctaccctaatggcgtaatggaagccaagatgcacgccaccggctacgtgcatgccacgttttacacacctgaaggactgagacacggcaccagactgcacacccacctgatcggaaacatccacacccacctggtgcattaccgggtggacctggatgtggctggaaccaagaacagcttccagacactccaaatgaagctggaaaacatcacaaacccttggagccccagacaccgggtggtgcagcctacccttgagcagacccagtacagctgggagagacaggccgcttttagatttaagagaaagctgccaaagtatctgctgttcaccagccctcaggagaacccctggggccataagcggacctacagactccagatccacagcatggccgaccaggtgcttcctcctggctggcaggaggaacaggccattacctgggctcggtaccccctggccgtgaccaagtacagagagagcgagctgtgcagcagctccatctaccaccagaacgacccttggcaccccccggtggttttcgagcaatttctgcacaacaacgagaatatcgagaacgaggatctggtggcatgggtcaccgtgggcttcctgcacatcccccactctgaggacatccctaataccgccacacctggcaatagcgtgggctttctgctgagacccttcaatttctttcctgaggaccctagcctggccagccgggacaccgtgatcgtgtggcctcgggacaacggcccaaactacgtgcaaagatggatccccgaagacagagattgcagcatgcctcctcctttctcctacaacggcacatatcggcctgtgtagtcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctccctatcgccacggcagaactcatcgccgcctgccttgcccgctgctggacaggggctaggttgctgggcactgataattccgtggtgttgtcggggaagctgacgtccattgccaccacctgtcaactcctttctgggactttcgctttccccctccctatcgccacggcagaactcatcgccgcctgccttgcccgctgctggacaggggctaggttgctgggcactgataattccgtggtgttgtcggggaagctgacgtccgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagcgctggctagaattacctaccggcctccaccataccttcgatattcgcgcccactctcccattaatccgcacaagtggatgtgatgcgattgcccgctaagatagttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 24 - DNA, 365 bp, Synthetic construct
  misc_feature 1..365: note = CMV enhancer
  source 1..365: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 25 - DNA, 248 bp, Synthetic construct
  misc_feature 1..248: note = CMV promoter
  source 1..248: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaat

SEQ ID NO: 26 - DNA, 509 bp, Synthetic construct
  misc_feature 1..509: note = 5' untranslated region
  source 1..509: mol_type = other DNA
actctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccag

SEQ ID NO: 27 - skipped

SEQ ID NO: 28 - DNA, 228 bp, Synthetic construct
  misc_feature 1..228: note = bovine growth hormone pA (BGHpA)
  source 1..228: mol_type = other DNA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 29 - DNA, 2256 bp, Synthetic construct
  misc_feature 1..2256: note = DAO1 (AOC1) ORF (codon optimized)
  source 1..2256: mol_type = other DNA
atgccggctcttggatgggccgttgccgcgatcctgatgctgcagacagccatggctgaacccagcccaggcaccctgcctagaaaggccggcgtgttcagtgatctgagcaaccaggagctgaaagctgtgcactcctttctctggtctaagaaggaactgagactgcagccatctagtaccactacaatggccaagaacaccgtgttcctgatcgagatgctgctgcccaagaaataccacgtgctgcggttcctggacaagggcgagagacatcctgtgcgggaagccagagctgtgattttcttcggagatcaggagcaccccaatgtaaccgaattcgccgtcggccctctgccaggcccatgctacatgcgggccctgtctcctcggcccggctaccagagctcttgggccagcagaccaatcagcaccgccgagtacgccctgctgtatcacacactgcaggaggccacaaaacctctgcaccagttcttcctgaacaccaccggcttcagcttccaggactgccacgacagatgtctggccttcacagacgtggcccctcggggagtggccagcggccaacggagatcttggctgatcatccagagatacgtcgagggatacttcctgcaccctacaggcctggaactgctggtcgaccacggaagcaccgacgccggccactgggccgtggaacaagtgtggtacaacggcaaattctacggttctccagaggagctggccagaaagtacgccgacggcgaagtggatgtggtggtgctcgaagatcctctgcctggcggaaagggccacgatagcacagaggaacctcctctgttcagctcccataagcccagaggcgacttcccctcgcccatccacgtgtccggccctagactggttcaacctcacggccccaggttccggctggagggcaacgccgtgctgtacggcggctggtccttcgccttcagactgagaagctcctccggcctgcaggtgctgaacgtgcactttggcggcgaaaggatcgcttacgaggtgtctgtgcaggaagctgtggccctgtacggcggccacacccctgccggcatgcagaccaaatacctggacgtgggatggggcctgggcagcgtgacacacgagctggcacctggcatcgattgccccgaaacagctaccttcctggatacctttcactactacgacgccgacgaccccgtccactaccctcgcgccctctgtctgttcgagatgcccaccggcgtgcctctgagaagacacttcaacagcaactttaagggcggattcaacttctacgcgggactgaagggccaggtgctggtgctgcgcaccacaagcaccgtctacaactatgactacatctgggacttcatcttctaccctaatggcgtaatggaagccaagatgcacgccaccggctacgtgcatgccacgttttacacacctgaaggactgagacacggcaccagactgcacacccacctgatcggaaacatccacacccacctggtgcattaccgggtggacctggatgtggctggaaccaagaacagcttccagacactccaaatgaagctggaaaacatcacaaacccttggagccccagacaccgggtggtgcagcctacccttgagcagacccagtacagctgggagagacaggccgcttttagatttaagagaaagctgccaaagtatctgctgttcaccagccctcaggagaacccctggggccataagcggacctacagactccagatccacagcatggccgaccaggtgcttcctcctggctggcaggaggaacaggccattacctgggctcggtaccccctggccgtgaccaagtacagagagagcgagctgtgcagcagctccatctaccaccagaacgacccttggcaccccccggtggttttcgagcaatttctgcacaacaacgagaatatcgagaacgaggatctggtggcatgggtcaccgtgggcttcctgcacatcccccactctgaggacatccctaataccgccacacctggcaatagcgtgggctttctgctgagacccttcaatttctttcctgaggaccctagcctggccagccgggacaccgtgatcgtgtggcctcgggacaacggcccaaactacgtgcaaagatggatccccgaagacagagattgcagcatgcctcctcctttctcctacaacggcacatatcggcctgtgtag

SEQ ID NO: 30 - DNA, 2312 bp, Homo sapiens
  source 1..2312: mol_type = genomic DNA
atgccggccctgggctgggccgtggctgccatcctgatgctgcagacggccatggcggagccctccccggggactctgcccaggaaggcaggggtgttttcagacctaagcaaccaagagctgaaggcagtgcacagcttcctctggtccaagaaggagctgaggctgcagccctccagtaccaccaccatggccaagaacaccgtgtttctcatcgagatgctgctgcccaagaagtaccatgtgctgaggtttctggataaaggtgaaaggcatcctgtgcgggaagcccgtgccgtcatcttctttggtgaccaggagcatcccaatgtcaccgagtttgctgtggggcccctgccagggccctgctacatgcgagcactgtcccccaggcctgggtaccagtcctcctgggcatcgaggcccatctccacagcagagtatgccctcctctaccacaccctgcaggaagccaccaagcccctgcatcagttcttcctcaataccacaggcttctcattccaagactgccatgacagatgcctggccttcaccgatgtggccccccggggtgtggcttctggccagcgccgcagttggcttatcatacagcgctatgtagaaggctactttctgcaccccactgggctggagctcctcgtggatcatgggagcacagatgctgggcactgggccgtggagcaggtgtggtacaacgggaagttctatgggagcccagaggaactggctcggaagtatgcagatggagaggtggacgtggtggtcctggaggacccgctgcctgggggcaaggggcatgacagcacagaggagccgcccctcttctcctcccacaagccccgcggggacttccccagccccatccatgtgagcggcccccgcttggtccagccccacggccctcgcttcaggctggagggcaacgctgtgctctacggcggctggagctttgccttccggctgcgctcctcctccgggctgcaggtcctgaacgtgcacttcggcggagagcgcattgcctatgaggtcagcgtgcaagaggcagtggcgctgtatggaggacacacacctgcaggcatgcagaccaagtacctcgatgtcggctggggcctgggcagcgtcactcatgagttagcccccggcatcgactgcccggagaccgccaccttcctggacactttccactactatgatgccgatgacccggtccattatccccgagccctctgcctctttgaaatgcccacaggggtgccccttcggcggcactttaattccaactttaaaggtggcttcaacttctatgcggggctgaagggccaggtgctggtgctgcggacaacttcaactgtctacaattatgattacatttgggactttatcttctaccccaacggggtgatggaggccaagatgcatgccactggctacgtccacgccaccttctacacccccgaggggctgcgccacggcactcgcctgcacacccacctgattggcaacatacacactcacttggtgcactaccgcgtagacctggatgtggcaggcaccaagaacagcttccagacactgcagatgaagctagaaaacatcaccaacccctggagcccaagacaccgcgtggtccagccaactctggagcagacgcagtactcctgggagcgccaggcggccttccgcttcaaaaggaagctgcctaagtacctgctctttaccagcccccaggagaacccctggggccacaagcgcacgtaccgcctgcagatccactccatggccgaccaggtgctgcccccaggctggcaggaggagcaggccatcacctgggcaaggactgaggggggccagcccagggccctgagccaagctgcttcgcctgtgcctggcaggtaccccctggcagtgaccaagtaccgggagtcggagctgtgcagcagcagcatctaccaccagaacgacccctggcacccgcccgtggtctttgagcagtttcttcacaacaacgagaacattgaaaatgaggacctggtggcctgggtgacggtgggcttcctgcacatcccccactcagaggacattcccaacacagccacacctgggaactccgtgggcttcctgctccggccattcaacttcttcccagaggacccctccctggcatccagagacactgtgatcgtgtggcctcgggacaacggccccaactacgtccagcgctggatccctgaggacagggactgctcgatgcctcccccttttagctacaatgggacctatagacctgtgtg

SEQ ID NO: 31 - DNA, 2255 bp, Homo sapiens
  source 1..2255: mol_type = genomic DNA
atgccggccctgggctgggccgtggctgccatcctgatgctgcagacggccatggcggagccctccccggggactctgcccaggaaggcaggggtgttttcagacctaagcaaccaagagctgaaggcagtgcacagcttcctctggtccaagaaggagctgaggctgcagccctccagtaccaccaccatggccaagaacaccgtgtttctcatcgagatgctgctgcccaagaagtaccatgtgctgaggtttctggataaaggtgaaaggcatcctgtgcgggaagcccgtgccgtcatcttctttggtgaccaggagcatcccaatgtcaccgagtttgctgtggggcccctgccagggccctgctacatgcgagcactgtcccccaggcctgggtaccagtcctcctgggcatcgaggcccatctccacagcagagtatgccctcctctaccacaccctgcaggaagccaccaagcccctgcatcagttcttcctcaataccacaggcttctcattccaagactgccatgacagatgcctggccttcaccgatgtggccccccggggtgtggcttctggccagcgccgcagttggcttatcatacagcgctatgtagaaggctactttctgcaccccactgggctggagctcctcgtggatcatgggagcacagatgctgggcactgggccgtggagcaggtgtggtacaacgggaagttctatgggagcccagaggaactggctcggaagtatgcagatggagaggtggacgtggtggtcctggaggacccgctgcctgggggcaaggggcatgacagcacagaggagccgcccctcttctcctcccacaagccccgcggggacttccccagccccatccatgtgagcggcccccgcttggtccagccccacggccctcgcttcaggctggagggcaacgctgtgctctacggcggctggagctttgccttccggctgcgctcctcctccgggctgcaggtcctgaacgtgcacttcggcggagagcgcattgcctatgaggtcagcgtgcaagaggcagtggcgctgtatggaggacacacacctgcaggcatgcagaccaagtacctcgatgtcggctggggcctgggcagcgtcactcatgagttagcccccggcatcgactgcccggagaccgccaccttcctggacactttccactactatgatgccgatgacccggtccattatccccgagccctctgcctctttgaaatgcccacaggggtgccccttcggcggcactttaattccaactttaaaggtggcttcaacttctatgcggggctgaagggccaggtgctggtgctgcggacaacttcaactgtctacaattatgattacatttgggactttatcttctaccccaacggggtgatggaggccaagatgcatgccactggctacgtccacgccaccttctacacccccgaggggctgcgccacggcactcgcctgcacacccacctgattggcaacatacacactcacttggtgcactaccgcgtagacctggatgtggcaggcaccaagaacagcttccagacactgcagatgaagctagaaaacatcaccaacccctggagcccaagacaccgcgtggtccagccaactctggagcagacgcagtactcctgggagcgccaggcggccttccgcttcaaaaggaagctgcctaagtacctgctctttaccagcccccaggagaacccctggggccacaagcgcacgtaccgcctgcagatccactccatggccgaccaggtgctgcccccaggctggcaggaggagcaggccatcacctgggcaaggtaccccctggcagtgaccaagtaccgggagtcggagctgtgcagcagcagcatctaccaccagaacgacccctggcacccgcccgtggtctttgagcagtttcttcacaacaacgagaacattgaaaatgaggacctggtggcctgggtgacggtgggcttcctgcacatcccccactcagaggacattcccaacacagccacacctgggaactccgtgggcttcctgctccggccattcaacttcttcccagaggacccctccctggcatccagagacactgtgatcgtgtggcctcgggacaacggccccaactacgtccagcgctggatccctgaggacagggactgctcgatgcctcccccttttagctacaatgggacctatagacctgtgtg

SEQ ID NO: 32 - DNA, 26 bp, synthetic construct
  source 1..26: mol_type = other DNA
actcccactgtcctttcctaataaaa

SEQ ID NO: 33 - DNA, 23 bp, synthetic construct
  source 1..23: mol_type = other DNA
cccccagaatagaatgacaccta

SEQ ID NO: 34 - DNA, 15 bp, synthetic construct
  source 1..15: mol_type = other DNA
ttgcatcgcattgtc